(19) United States
(12) Patent Application Publication
(10) Pub. No.: US 2023/0392180 A1
(43) Pub. Date: Dec. 7, 2023

(54) MICROSCOPIC IMAGING AND ANALYSES OF EPIGENETIC LANDSCAPE

(71) Applicant: Sanford Burnham Prebys Medical Discovery Institute, La Jolla, CA (US)

(72) Inventor: Alexey V. TERSKIKH, Solana Beach, CA (US)

(21) Appl. No.: 18/327,715

(22) Filed: Jun. 1, 2023

Related U.S. Application Data
(63) Continuation of application No. PCT/US2021/061878, filed on Dec. 3, 2021.
(60) Provisional application No. 63/121,477, filed on Dec. 4, 2020.

(51) Int. Cl. C12Q 1/02; G16B 30/00

(52) U.S. Cl. CPC C12Q 1/025; G16B 30/00; C12Q 2600/158

(57) ABSTRACT

The present disclosure relates to immunofluorescence detection of epigenetic markers and automated cell imaging by a machine learning to profile and quantify the “epigenetic state” of individual cells.

### CROSS-REFERENCE

This application is a continuation of International Patent Application No. PCT/US2021/061878, filed Dec. 3, 2021, which claims the benefit of U.S. Provisional Patent Application No. 63/121,477, filed Dec. 4, 2020, each of which is incorporated by reference herein in its entirety.

### FIELD OF THE DISCLOSURE

The present disclosure relates generally to epigenetic states of cells.

### BACKGROUND OF THE DISCLOSURE

Epigenetic modifications have been established as a powerful universal mechanism to regulate gene expression and cell fates during development and in adult organisms. Aberrant regulation of epigenetic maintenance has been implicated in cancer, including tumor initiating/propagating cells. A cell's epigenetic landscape is largely determined by its chromatin organization, the pattern of its DNA, and its histone modifications, all of which confer differential accessibility to areas of the genome and, through direct and indirect regulation of all DNA-related processes, form the basis of the cellular phenotype. By collecting global information about the epigenetic landscape, for example using ATAC- or histone ChIP-seq, multilayered information regarding cellular states could be derived including stable cell phenotypes such as quiescence, senescence, or cell fate, as well as transient changes such as those induced by cytokines and chemical compounds.

Yet, traditional methods, including ATAC- or histone ChIP-seq, are limited to a technique requiring several millions of cells per experiment, thus could not be effectively used to determine substantial cell-to-cell variations are observed within every normal tissue and within malignancies. Further, those methods are not well adapted for high-content drug screening. Finally, a fundamental limitation of all of such methods is that the cell in question is inevitably destroyed in the process of acquiring the measurement. More recently, novel image analysis coupled with multiparametric analysis and machine learning have significantly impacted the ability to understand and process phenotypic screening outputs. Yet, such image analysis has not been adapted to extract and utilize information for the cellular epigenetic landscape.

### SUMMARY OF THE DISCLOSURE

The present disclosure includes methods relating to microscopic imaging and epigenetic analysis of cells at a single cell level and applications of the epigenetic analysis in determining biological clock and/or effect of external stimulus (e.g., environmental toxins or drugs) to cell aging or rejuvenation. Thus, the present disclosure includes a method of determining a biological age of a primary cell at a single cell level. The method comprises steps of 1) detecting expression patterns of a plurality of epigenetic marks in a first plurality of primary cells and a second plurality of primary cells, wherein the first and second plurality of primary cells belong to first and second biological entities, respectively, wherein the first and second biological entities are associated with predetermined first and second chronological ages, respectively; 2) determining multiparametric signatures of the first and second plurality of primary cells based at least in part on the expression patterns of the plurality of epigenetic marks; and 3) comparing the multiparametric signatures of the first plurality of primary cells and the multiparametric signatures of the second plurality of primary cells by plotting against the first and second chronological ages, and 4) determining the biological age of the primary cell at least based in part on the comparison of the multiparametric signatures. In certain instances, detecting the expression pattern comprises detection of chromatin shape, detection of a DNA modification, detection of a histone modification, or a combination thereof.

In some aspects, the present disclosure relates generally to imaging of epigenetic states of single cells in cultures or within the tissues, including tissue sections, and more specifically to methods determining the epigenetic signature (e.g., pattern) of stem cells, including pluripotent stem cells, multipotent progenitors, committed cells, and various terminally differentiation states (including transformed or neoplastic state i.e. cancer) across different ages of a cell using immunofluorescence or fluorescence imaging in live cells based on genetically engineered epigenetic probes.

In some instances, detecting expression patterns of the plurality of epigenetic marks in the first plurality of primary cells and the second plurality of primary cells comprises detecting expression patterns of a plurality of epigenetic marks in a nucleus of a cell in the first plurality of primary cells and in a nucleus of a cell in the second plurality of primary cells. In some instances, expression patterns comprises detection of chromatin shape, detection of a DNA modification, detection of a nuclear staining pattern, detection of a histone modification, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of H3K4me3, H3K4me1, H3K27me3, H3K27ac, H3K9me3, H3K9ac, H4K12ac, H4K12me3, H3K18ac, and any combinations thereof. Alternatively, and/or additionally, the multiparametric signature comprises a texture-associated feature. In certain instances the texture-associated feature comprises Haralick texture features, threshold adjacency statistics, radial features, Gabor related features, or other types of texture features and any combinations thereof.

It is contemplated that the texture-associated feature is analyzed using a subcellular feature analysis, a machine feature extraction algorithm, a machine learning protocol, or a combination thereof. In some instances, the machine learning protocol comprises a machine learning algorithm selected from a group consisting of support vector machine, support vector regression, a linear regression, a quadratic discriminant analysis, a neural network, or a combination thereof. In some instances, the machine learning protocol is quadratic discriminant analysis, and wherein the multiparametric signature of the plurality of the first primary cells are used to determine a cell population of the first plurality of primary cells from multiple cell populations. In some instances, the machine learning protocol is support vector machine, and wherein the multiparametric signature of the plurality of the first primary cells is used to distinguish and/or determine a character of the first plurality of primary cells in a single cell population.

In some instances, the method further comprises determining a relationship between the first plurality of primary cells and the second plurality of primary cells by comparing the multiparametric signatures. In some instances, comparing the multiparametric signatures comprises determining a first centroid of a first element of the multiparametric signature of the first plurality of primary cells and a second centroid of the first element of the multiparametric signature of the second plurality of primary cells. In certain instances, the comparing the multiparametric signatures comprises determining a plurality of centroids of each of the multiparametric signatures of the first plurality of primary cells and the second plurality of primary cells. In some instances, the method further comprises comprising calculating a first multivariant centroid of the first plurality of primary cells and a second multivariant centroid of the second plurality of primary cells. In some instances, the method further comprises plotting the first multivariant centroid and the second multivariant centroid relative to the chronological ages of the first and second plurality of primary cells.

In some instances, the relation is determined by calculating Euclidian distance between the first primary cells and the second plurality of primary cells using machine learning based on the multiparametric signatures. In other instances, the method further comprises reducing a data dimensionality of the expression pattern. It is contemplated that the expression pattern comprises 3-dimensional topological distribution, and wherein the reducing the data dimensionality comprises interpreting the 3-dimensional topological distribution as a two-dimensional projection using a multidimensional scaling. In some instances, the two-dimensional projection comprises a scattered plot. In some instances, the first plurality of primary cells and the second plurality of primary cells are the same type of cells. It is contemplated that the first plurality of primary cells and the second plurality of primary cells are different cell types. In some instances the cell type comprises a hepatocyte, a fibroblast, a peripheral blood mononuclear cell, or an immune cell.

In some instances, that the first plurality of primary cells and the second plurality of primary cells are isolated and/or derived from different tissues, and the method further comprises determining a relationship between the different tissues. In some instances, the first plurality of primary cells and the second plurality of primary cells are isolated and/or derived from different organs, and the method further comprises determining a relationship between the different organs. Alternatively and/or additionally, the first and second biological entities are isolated and/or derived from a same organ or a same individual. In some instances, the first plurality of primary cells and the second plurality of primary cells are isolated from different individual in different ages.

It is contemplated that the multiparametric signature comprises a multiparametric signature of a centroid of the first plurality of primary cells, and the method further comprises determining a relationship between the centroid of the first plurality of primary cells and the centroid of the second plurality of primary cells by comparing the multiparametric signatures. In other instances, biological age of the primary cell is determined based on the plotting the first multivariant centroid and the second multivariant centroid. In some instances, detecting expression patterns of the plurality of epigenetic marks in the first plurality of primary cells and the second plurality of primary cells comprises capturing a series of images of the first plurality of primary cells and the second plurality of primary cells over a period of time. In some instances, capturing a series of images of first plurality of primary cells and the second plurality of primary cells over a period of time comprises capturing at least one image of first plurality of primary cells and the second plurality of primary cells before, during, and after mitosis.

The present disclosure also includes methods of determining an effect of a treatment on a primary cell, comprising the steps of 1) applying the treatment to a primary cell to obtain a treated primary cell; 2) detecting expression patterns of a plurality of epigenetic marks in the treated primary cell and an untreated primary cell; 3) determining a multiparametric signatures of the treated and untreated primary cells based on the expression patterns of the plurality of epigenetic marks; and 4) comparing the multiparametric signature of the treated primary cell to a multiparametric signature of the untreated primary cell, thereby determining the effect of the treatment on the primary cell based at least in part on the comparing of the multiparametric signatures. In some instances, the treatment comprises an exposure to a small molecule, radiation, light, temperature, an environmental exposure, or a combination thereof. In some instances, detecting expression patterns of a plurality of epigenetic marks in the treated primary cell and an untreated primary cell comprises detecting expression patterns of a plurality of epigenetic marks in a nucleus of the treated primary cell and in a nucleus of the untreated primary cell. It is contemplated that detecting expression pattern comprises detection of chromatin shape, detection of a DNA modification, detection of a histone modification, detection of a nuclear staining pattern, detection of one or more genetically encoded epigenetic probes, or a combination thereof. In some instances, the histone modification is selected from the group consisting of H3K4me3, H3K4me1, H3K27me3, H3K27ac, H3K9me3, H3K9ac, H4K12ac, H4K12me3, H3K18ac, and any combinations thereof. In some instances, detecting expression patterns of a plurality of epigenetic marks in the treated primary cell and an untreated primary cell comprises detecting expression patterns of a plurality of epigenetic marks in a nucleus of the treated primary cell and in a nucleus of the untreated primary cell.

It is contemplated that the multiparametric signature comprises a texture-associated feature. In some instances, the texture-associated feature comprises Haralick texture features, threshold adjacency statistics, radial features, Gabor related features, or other types of texture features and any combinations thereof. In some instances, the texture-associated feature is analyzed using a subcellular feature analysis, a machine learning protocol, or a combination thereof. In some instances, the machine learning protocol comprises a machine learning algorithm selected from the group consisting of support vector machine, support vector regression, a linear regression, a quadratic discriminant analysis, or any combinations thereof. In some instances, the method further comprises Euclidian distance of the treated primary cell using machine learning based on the epigenetic signature of the treated primary cell.

In some instances, the primary cell comprises a hepatocyte, a fibroblast, a peripheral blood mononuclear cell, a lymphocyte, a stem cell, a progenitor cell, a fetal cell, an embryonic stem cell (ESC), an induced pluripotent cell (iPSC), a neural stem/precursor cell, a neuron, an astrocyte, a smooth muscle cell, or a tumor cell. In some instances, the change of the multiparametric signature of the treated primary cell is related to cell aging, cell sensitivity, cell metabolism, cell signaling, cell cycle, or cell health. It is contemplated that the change of the multiparametric signature of the treated primary cell is related to cell aging, wherein the multiparametric signature of the treated primary cell is similar to an epigenetic signature of a primary cell at a younger age, and wherein the effect of the treatment on the primary cell is identified as an anti-aging or rejuvenating. In some instances, the treatment to the primary cell induces an epigenetic change without exerting cytotoxicity proportional to the epigenetic change to the primary cell.

In some instances, the epigenetic change is determined by comparing the epigenetic change of the treated primary cell to an epigenetic change of a primary cell treated with a known epigenetically active compound. It is contemplated that the known epigenetically active compound comprises a HDAC inhibitor, an EZH2 inhibitor, an AURORA kinase inhibitor, a SIRT inhibitor, or any combinations thereof. In some instances, the treatment is identified as an environmental toxin. In some instances, the change of the epigenetic signature of the treated primary cell is related to cell cycle, and wherein the effect of the treatment on the primary cell is identified as cell senescence induction. It is contemplated that the treated primary cell and untreated primary cell are obtained from a same tissue or a same organ of a subject. In some instances, the method further comprises determining the effect of the treatment to the tissue or the organ.

In some instances, determining the effect of the treatment on the primary cell based at least in part on the comparing of the multiparametric signatures comprises determining the effect on the biological age of the primary cell and/or determining the effect on cell division. In some instances, detecting the expression patterns of the plurality of epigenetic marks in the treated primary cell and the untreated primary cell comprises capturing a series of images of the treated primary cell and the untreated primary cell over a period of time. In some instances, capturing a series of images of the treated primary cell and the untreated primary cell over a period of time comprises capturing at least one image of the treated primary cell and the untreated primary cell before, during, and after mitosis.

In some instances, the method further comprises applying another treatment to another primary cell to obtain another treated primary cell; detecting expression patterns of a plurality of epigenetic marks in the another treated primary cell; determining a multiparametric signatures of the another treated primary cells based on the expression patterns of the plurality of epigenetic marks; and comparing the multiparametric signatures of the treated primary cell, the another treated primary cell, and the untreated primary cell, thereby determining the effect of the different treatments on the primary cell based at least in part on the comparing of the multiparametric signatures. In some instances, that the multiparametric signature comprises a multiparametric signature of a centroid of the treated primary cell, and the method further comprises determining a relationship between the centroid of the treated primary cell and the centroid of the untreated primary cell by comparing the multiparametric signatures. In some instances, the treatment is a drug candidate. It is contemplated that the drug candidate is an inducing reagent of cellular rejuvenation. In some instances, the effect of the treatment is a change of a biological age of the treated primary cell determined based on the multiparametric signature of the treated primary cell compared to the multiparametric signature of the untreated primary cell.

In some aspects, the present disclosure provides methods of determining a biological age of a primary cell at a single cell level comprising: detecting expression patterns of a plurality of epigenetic marks in a first plurality of primary cells and a second plurality of primary cells, wherein the first and second plurality of primary cells are associated with predetermined first and second chronological ages, respectively; determining multiparametric signatures of the first and second plurality of primary cells based at least in part on the expression patterns of the plurality of epigenetic marks; comparing the multiparametric signatures of the first plurality of primary cells and the multiparametric signatures of the second plurality of primary cells by plotting against the first and second chronological ages; and determining the biological age of the primary cell at least based in part on the comparison of the multiparametric signatures.

In some instances, detecting expression patterns of the plurality of epigenetic marks in the first plurality of primary cells and the second plurality of primary cells comprises detecting expression patterns of a plurality of epigenetic marks in a nucleus of a cell in the first plurality of primary cells and in a nucleus of a cell in the second plurality of primary cells. In some instances, detecting the expression patterns comprises detection of chromatin shape, detection of a DNA modification, detection of a nuclear staining pattern, detection of a histone modification, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combination thereof. In some instances, the multiparametric signature comprises a texture-associated feature. In some instances, the texture-associated feature comprises Haralick texture features, threshold adjacency statistics, radial features, Gabor related features, or other types of texture features and a combination thereof. In some instances, the texture-associated feature is analyzed using a subcellular feature analysis, a machine feature extraction algorithm, a machine learning protocol, or a combination thereof.

In some instances, the machine learning protocol comprises a machine learning algorithm selected from a group consisting of support vector machine, support vector regression, a linear regression, a quadratic discriminant analysis, a neural network, or a combination thereof. In some instances, the machine learning protocol is quadratic discriminant analysis, and wherein the multiparametric signature of the first plurality of the primary cells is used to distinguish a cell population of the first plurality of primary cells from multiple cell populations. In some instances, the machine learning protocol is support vector machine, and wherein the multiparametric signature of the plurality of the first primary cells is used to identify a character of the first plurality of primary cells in a single cell population.

In some instances, the methods described herein further comprise determining a relationship between the first plurality of primary cells and the second plurality of primary cells by comparing the multiparametric signatures. In some instances, comparing the multiparametric signatures comprises determining a first centroid of a first element of the multiparametric signature of the first plurality of primary cells and a second centroid of the first element of the multiparametric signature of the second plurality of primary cells. In some instances, comparing the multiparametric signatures comprises determining a plurality of centroids of each of the multiparametric signatures of the first plurality of primary cells and the second plurality of primary cells. In some instances, the methods described herein further comprise calculating a first multivariant centroid of the first plurality of primary cells and a second multivariant centroid of the second plurality of primary cells. In some instances, the methods described herein further comprise plotting the first multivariant centroid and the second multivariant centroid relative to the chronological ages of the first and second plurality of primary cells. In some instances, the relation is determined by calculating Euclidian distance between the first plurality of primary cells and the second plurality of primary cells using machine learning based on the multiparametric signatures.

In some instances, the methods described herein further comprise reducing a data dimensionality of the expression pattern. In some instances, the expression pattern comprises 3-dimensional topological distribution, and wherein the reducing the data dimensionality comprises interpreting the 3-dimensional topological distribution as a two-dimensional projection using a multidimensional scaling. In some instances, the two-dimensional projection comprises a scattered plot.

In some instances, the first plurality of primary cells and the second plurality of primary cells comprises same type of cells. In some instances, the first plurality of primary cells and the second plurality of primary cells comprises different type of cells. In some instances, the type cell comprises a hepatocyte, a fibroblast, a peripheral blood mononuclear cell, or an immune cell. In some instances, the first plurality of primary cells and the second plurality of primary cells are isolated from different tissues, and the method further comprises determining a relationship between the different tissues. In some instances, the first plurality of primary cells and the second plurality of primary cells are isolated from different organs, and the method further comprises determining a relationship between the different organs.

In some instances, the first and second biological entities are derived from a same tissue, a same organ, or a same individual. In some instances, the first plurality of primary cells and the second plurality of primary cells are isolated from different individuals in different ages. In some instances, the multiparametric signature comprises a multiparametric signature of a centroid of the first plurality of primary cells, and the method further comprises determining a relationship between the centroid of the first plurality of primary cells and the centroid of the second plurality of primary cells by comparing the multiparametric signatures. In some instances, the biological age of the primary cell is determined based on the plotting the first multivariant centroid and the second multivariant centroid. In some instances, detecting expression patterns of the plurality of epigenetic marks in the first plurality of primary cells and the second plurality of primary cells comprises capturing a series of images of the first plurality of primary cells and the second plurality of primary cells over a period of time. In some instances, capturing a series of images of first plurality of primary cells and the second plurality of primary cells over a period of time comprises capturing at least one image of first plurality of primary cells and the second plurality of primary cells before, during, and after mitosis.

In some aspects, the present disclosure also includes methods of determining an effect of a treatment on a primary cell, comprising applying the treatment to a primary cell to obtain a treated primary cell; detecting expression patterns of a plurality of epigenetic marks in the treated primary cell and an untreated primary cell; determining at least one multiparametric signature of the treated and untreated primary cells based at least in part on the expression patterns of the plurality of epigenetic marks; and comparing the multiparametric signature of the treated primary cell to a multiparametric signature of the untreated primary cell; and determining the effect of the treatment on the primary cell based at least in part on the comparing of the multiparametric signatures. In some instances, detecting expression patterns of a plurality of epigenetic marks in the treated primary cell and an untreated primary cell comprises detecting expression patterns of a plurality of epigenetic marks in a nucleus of the treated primary cell and in a nucleus of the untreated primary cell. In some instances, the treatment comprises an exposure to a small molecule, radiation, light, temperature, an environmental exposure, or a combination thereof. In some instances, detecting expression pattern comprises detection of chromatin shape, detection of a DNA modification, detection of a histone modification, detection of a nuclear staining pattern, detection of one or more genetically encoded epigenetic probes, or a combination thereof. In some instances, the histone modification is selected from the group consisting of H3K4me3, H3K4me1, H3K27me3, H3K27ac, H3K9me3, H3K9ac, H4K12ac, H4K12me3, H3K18ac, and any combinations thereof. In some instances, the multiparametric signature comprises a texture-associated feature. In some instances, the texture-associated feature comprises Haralick texture features, threshold adjacency statistics, radial features, Gabor related features, or other types of texture features and any combinations thereof. In some instances, the texture-associated feature is analyzed using a subcellular feature analysis, a machine learning protocol, or a combination thereof.

In some instances, the machine learning protocol comprises a machine learning algorithm selected from the group consisting of support vector machine, support vector regression, a linear regression, a quadratic discriminant analysis, or any combinations thereof. In some instances, the methods described herein further comprises calculating Euclidian distance of the treated primary cell using machine learning based on the epigenetic signature of the treated primary cell. In some instances, the primary cell comprises a hepatocyte, a fibroblast, a peripheral blood mononuclear cell, a lymphocyte, a stem cell, a progenitor cell, a fetal cell, an embryonic stem cell (ESC), an induced pluripotent cell (iPSC), a neural stem/precursor cell, a neuron, an astrocyte, a smooth muscle cell, or a tumor cell. In some instances, the change of the multiparametric signature of the treated primary cell is related to cell aging, cell sensitivity, cell metabolism, cell signaling, cell cycle, or cell health. In some instances, the change of the multiparametric signature of the treated primary cell is related to cell aging, wherein the multiparametric signature of the treated primary cell is similar to an epigenetic signature of a primary cell at a younger age, and wherein the effect of the treatment on the primary cell is identified as an anti-aging or rejuvenating. In some instances, the treatment to the primary cell induces an epigenetic change without exerting cytotoxicity proportional to the epigenetic change to the primary cell.

In some instances, the epigenetic change is determined by comparing the epigenetic change of the treated primary cell to an epigenetic change of a primary cell treated with a known epigenetically active compound. In some instances, the known epigenetically active compound comprises a HDAC inhibitor, an EZH2 inhibitor, an AURORA kinase inhibitor, a SIRT inhibitor, or any combinations thereof. In some instances, the treatment is identified as an environmental toxin. In some instances, the change of the epigenetic signature of the treated primary cell is related to cell cycle, and wherein the effect of the treatment on the primary cell is identified as cell senescence induction.

In some instances, the treated primary cell and untreated primary cell are obtained from a same tissue or a same organ of a subject. In some instances, the methods described herein further comprise determining the effect of the treatment to the tissue or the organ. In some instances, determining the effect of the treatment on the primary cell based at least in part on the comparing of the multiparametric signatures comprises determining the effect on the biological age of the primary cell and/or determining the effect on cell division. In some instances, detecting the expression patterns of the plurality of epigenetic marks in the treated primary cell and the untreated primary cell comprises capturing a series of images of the treated primary cell and the untreated primary cell over a period of time. In some instances, capturing a series of images of the treated primary cell and the untreated primary cell over a period of time comprises capturing at least one image of the treated primary cell and the untreated primary cell before, during, and after mitosis.

In some instances, the methods described herein further comprise applying another treatment to another primary cell to obtain another treated primary cell; detecting expression patterns of a plurality of epigenetic marks in the another treated primary cell; determining a multiparametric signatures of the another treated primary cells based on the expression patterns of the plurality of epigenetic marks; comparing the multiparametric signatures of the treated primary cell, the another treated primary cell, and the untreated primary cell; and determining the effect of the different treatments on the primary cell based at least in part on the comparing of the multiparametric signatures. In some instances, the multiparametric signature comprises a multiparametric signature of a centroid of the treated primary cell, and the method further comprises determining a relationship between the centroid of the treated primary cell and the centroid of the untreated primary cell by comparing the multiparametric signatures. In some instances, the treatment is an exposure to a drug candidate molecule. In some instances, the drug candidate molecule is an inducing reagent of cellular rejuvenation. In some instances, the effect of the treatment is a change of a biological age of the treated primary cell determined based on the multiparametric signature of the treated primary cell compared to the multiparametric signature of the untreated primary cell.

In some aspects, the present disclosure also includes methods of detecting aging of a biological entity, comprising: detecting expression patterns of a plurality of epigenetic marks in a plurality of primary cells of a biological entity; determining multiparametric signatures of the plurality of primary cells based on the expression patterns of the plurality of epigenetic marks; determining average value of coefficient of variance of multiparametric signatures of the plurality of primary cells; and determining the aging of the biological entity based at least in part on the average value of coefficient of variance of multiparametric signatures. In some instances, detecting expression patterns of a plurality of epigenetic marks in a plurality of primary cells of a biological entity comprises detecting expression patterns of a plurality of epigenetic marks in a nucleus of the plurality of the primary cells. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the multiparametric signature comprises a texture-associated feature. In some instances, the texture-associated feature comprises Haralick texture features, threshold adjacency statistics, radial features, Gabor related features, or other types of texture features and any combination thereof. In some instances, the texture-associated feature is analyzed using a subcellular feature analysis, a machine feature extraction algorithm, a machine learning protocol, or a combination thereof. In some instances, increased average value of coefficient of variance of multiparametric signatures indicates onset of aging of the biological entity. In some instances, increased average value of coefficient of variance of multiparametric signatures indicates increased biological age of the biological entity. In some instances, the biological entity is a tissue, an organ, or an organism. In some instances, the biological entity is a diseased tissue, a diseased organ, or a diseased organism. In some instances, the primary cell is a hepatocyte, a fibroblast, a peripheral blood mononuclear cell, or an immune cell. In some instances, the primary cell is a cell treated with a drug, a chemical agent, or has been exposed to an environmental toxin, radiation, light, or temperature. In some instances, detecting expression patterns of the plurality of epigenetic marks in a plurality of primary cells of a biological entity comprises capturing a series of images of the plurality of primary cells over a period of time. In some instances, capturing a series of images of the plurality of primary cells over a period of time comprises capturing at least one image of the plurality of primary cells before, during, and after mitosis.

In some aspects, the present disclosure also provides methods of predicting a residual lifespan of a biological entity, comprising: detecting expression patterns of a plurality of epigenetic marks in a first plurality of primary cells and in a second plurality of primary cells of a biological entity, wherein the first and second plurality of primary cells are associated with different chronological ages; determining multiparametric signatures of the first and second plurality of primary cells based on the expression patterns of the plurality of epigenetic marks; determining average values of coefficient of variance of multiparametric signatures of the first and second plurality of primary cells; and predicting the residual lifespan of the biological entity based at least in part on the changes of the average values of coefficient of variance of multiparametric signatures. In some instances, detecting expression patterns of the plurality of epigenetic marks in the first plurality of primary cells and the second plurality of primary cells comprises detecting expression patterns of a plurality of epigenetic marks in a nucleus of a cell in the first plurality of primary cells and in a nucleus of a cell in the second plurality of primary cells. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the multiparametric signature comprises a texture-associated feature. In some instances, the texture-associated feature comprises Haralick texture features, threshold adjacency statistics, radial features, Gabor related features, or other types of texture features and any combination thereof. In some instances, the chronological ages of the first and second plurality of primary cells are at least 60 days, 90 days, 120 days, 180 days, 1 year, 2 years, 3 years different from each other. In some instances, the residual lifespan of the biological entity is predicted by a function of CV=c−a/(x−b). In some instances, the biological entity is a tissue, an organ, or an organism.

In some aspects, the present disclosure also provides methods of improving accuracy of imaging analysis of a primary cell at a single cell level comprising: detecting expression patterns of a plurality of epigenetic marks in a primary cell; determining co-occurrence of the expression patterns of the plurality of epigenetic marks and chromatin density; and determining multiparametric signature of the primary cell based at least in part on the co-occurrence of the expression patterns, thereby improving accuracy of imaging analysis of the primary cell. In some instances, detecting expression patterns of a plurality of epigenetic marks in a nucleus of a primary cell comprises detecting expression patterns of a plurality of epigenetic marks in a nucleus of the primary cell. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the multiparametric signature comprises a texture-associated feature. In some instances, the texture-associated feature comprises Haralick texture features, threshold adjacency statistics, radial features, Gabor related features, or other types of texture features and any combination thereof. In some instances, the co-occurrence comprises frequency or spatial distribution of the expression patterns that cooccur in the nucleus of the primary cell. In some instances, the co-occurrence comprises frequency and spatial distribution of the expression patterns that cooccur in the nucleus of the primary cell. In some instances, the methods described herein further comprise quantifying first and second pixel values of first and second epigenetic marks at a plurality of locations in the nucleus of the primary cell and first and second probabilities of density of the first and second epigenetic marks at the plurality of locations.

In some aspects, the present disclosure also provides methods of determining an effect of a treatment on a primary cell, comprising: applying the treatment to a primary cell to obtain a treated primary cell; detecting expression patterns of a plurality of epigenetic marks in the treated primary cell and an untreated primary cell; determining co-occurrence of the expression patterns of the plurality of epigenetic marks and chromatin density; and determining the effect of the treatment on the primary cell based at least in part on a difference of co-occurrence of the expression patterns between treated primary cell and the untreated primary cell. In some instances, detecting expression patterns of a plurality of epigenetic marks in the treated primary cell and an untreated primary cell comprises detecting expression patterns of a plurality of epigenetic marks in a nucleus of the treated primary cell and in a nucleus of the untreated primary cell. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the treatment comprises an exposure to a small molecule, radiation, light, temperature, an environmental exposure, or a combination thereof. In some instances, the primary cell comprises a hepatocyte, a fibroblast, a peripheral blood mononuclear cell, a lymphocyte, a stem cell, a progenitor cell, a fetal cell, an embryonic stem cell (ESC), an induced pluripotent cell (iPSC), a neural stem/precursor cell, a neuron, an astrocyte, a smooth muscle cell, or a tumor cell. In some instances, co-occurrence of the expression patterns comprises frequency or spatial distribution of the expression patterns that cooccur in the nucleus of the primary cell. In some instances, the co-occurrence comprises frequency and spatial distribution of the expression patterns that cooccur in the nucleus of the primary cell.

In some instances, the methods described herein further comprise quantifying first and second pixel values of first and second epigenetic marks at a plurality of locations in the nucleus of the primary cell and first and second probabilities of density of the first and second epigenetic marks at the plurality of locations. In some instances, the difference of the co-occurrence comprises a difference in frequency or spatial distribution of the expression patterns. In some instances, determining the effect of the treatment on the primary cell based at least in part on the comparing of the multiparametric signatures comprises determining the effect on the biological age of the primary cell and/or determining the effect on cell division. In some instances, detecting expression patterns of a plurality of epigenetic marks in a plurality of primary cells of a biological entity comprises detecting expression patterns of a plurality of epigenetic marks in a nucleus of the plurality of the primary cells comprises capturing a series of images of the plurality of the primary cells over a period of time. In some instances, capturing a series of images of the plurality of the primary cells over a period of time comprises capturing at least one image of the plurality of the primary cells before, during, and after mitosis.

In some instances, detecting the expression patterns (e.g., detection of chromatin shape, detection of a DNA modification, detection of a nuclear staining pattern, detection of a histone modification, detection of one or more genetically encoded epigenetic probes, or a combination thereof) is done using confocal microscopy and the intensity of all voxels in all channels is recorded so that the entire image could be accurately reconstructed from such tables holding the digital information about the image voxels. In some instances, chromatin density is determined by DAPI staining or equivalent methods reporting chromatin density in 3D or in 2D projections. In some instances, epigenetic marks comprise DNA methylation.

### INCORPORATION BY REFERENCE

All publications, patents, and patent applications mentioned in this specification and exhibits are herein incorporated by reference to the same extent as if each individual publication, patent, or patent application was specifically and individually indicated to be incorporated by reference.

### DETAILED DESCRIPTION OF THE DISCLOSURE

### Definitions

As used in the specification and appended claims, unless specified to the contrary, the following terms have the meaning indicated below.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some instances of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Throughout this disclosure, various instances are presented in a range format. It should be understood that the description in range format is merely for convenience and brevity and should not be construed as an inflexible limitation on the scope of any instances. Accordingly, the description of a range should be considered to have specifically disclosed all the possible subranges as well of any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure unless the context clearly dictates otherwise.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, “treatment of” or “treating,” “applying”, or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some instances, administered to a patient at risk of developing a particular disease or condition, or to a patient reporting one or more of the physiological symptoms of a disease, even though a diagnosis of this disease has not been made.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The biological entity can be an organ, a tissue, a cell, a plurality of cells, a cell population, or its progeny from an individual organism, containing multiple distinct biological entities within similar tissues, cells, and their progeny. The subject can be tissues, cells and their progeny of a biological entity obtained in vivo or cultured in vitro. The subject can be a mammal. The mammal can be a human. The subject may be diagnosed or suspected of being at high risk for a disease or any pathological condition. In some cases, the subject is not necessarily diagnosed or suspected of being at high risk for the disease.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range. In certain instances, the term “about” also includes the exact number (i.e., about 5 includes 5).

As used herein, “Embryonic germ cells” or “EG cells” are cells derived from the primordial germ cells of an embryo or fetus that are destined to give rise to sperm or eggs. EG cells are among the embryonic stem cells that can be cultured in accordance with the disclosure. “Embryonic stem cells” or “ES cells” are cells obtained from an animal (e.g., a primate, such as a human) embryo, preferably from an embryo that is less than about eight weeks old. Preferred embryonic stages for isolating primordial embryonic stem cells include the morula or blastocyst stage of a pre-implantation stage embryo. Well-known criteria for characterizing a cell as a stem cell are intended herein. See, e.g., Hoffman and Carpenter, Nature Biotech. 23:699-708, 2005, for a listing of such criteria.

“Extracellular matrix” (ECM) or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. The ECM thus encompasses essentially all secreted molecules that are immobilized outside of the cell. In vivo, the ECM provides order in the extracellular space and serves functions associated with establishing, separating, and maintaining differentiated tissues and organs. The ECM is a complex structure that is found, for example, in connective tissues and basement membranes, also referred to as the basal lamina. Connective tissue typically contains isolated cells surrounded by ECM that is naturally secreted by the cells. Components of the ECM have been shown to interact with and/or bind growth and differentiation factors, cytokines, matrix metalloproteases (MMPs), tissue inhibitors of metalloproteases (TIMPs), and other soluble factors that regulate cell proliferation, migration, and differentiation. Descriptions of the ECM and its components may be found in, among other places, Guidebook to the Extracellular Matrix, Anchor, and Adhesion Proteins, 2d ed., Kreis and Vale, eds., Oxford University Press, 1999 (“Kreis et al.”); Geiger et al., Nature Reviews Molecular Cell Biology 2:793-803, 2001; Iozzo, Annual Review of Biochemistry, 1998, Annual Reviews, Palo Alto, Calif; Boudreau and Jones, Biochem. J. 339:481-88, 1999; Extracellular Matrix Protocols, Streuli and Grant, eds., Humana Press 2000; Metzler, Biochemistry the Chemical Reactions of Living Cells, 2d ed., vol. 1, 2001, Academic Press, San Diego, particularly chapter 8; and Lanza et al., particularly chapters 4 and 20.

“Pluripotent” refers to cells that are capable of differentiating into one of a plurality of different cell types, although not necessarily all cell types. An exemplary class of pluripotent cells is embryonic stem cells, which are capable of differentiating into any cell type in the human body. Thus, it will be recognized that while pluripotent cells can differentiate into multipotent cells and other more differentiated cell types, the process of reverse differentiation (i.e., de-differentiation) is likely more complicated and requires “re-programming” the cell to become more primitive, meaning that, after re-programming, it has the capacity to differentiate into more or different cell types than was possible prior to re-programming.

“Stem cell” includes any stem or precursor cell, whether from a human or non-human source, and cells derived from stem cells that retain characteristics of precursor cells.

“Cancer” or “tumor” refers to various types of malignant neoplasms and tumors, including primary tumors, and tumor metastasis. Non-limiting examples of cancers which can be detected by the sensor array and system of the present disclosure are brain, ovarian, colon, prostate, kidney, bladder, breast, lung, oral, and skin cancers. Specific examples of cancers are: carcinomas, sarcomas, myelomas, leukemias, lymphomas and mixed type tumors. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

“Substantially undifferentiated” means that population of stem cells (e.g., primate primordial stem cells) contains at least about 50%, preferably at least about 60%, 70%, or 80%, and even more preferably, at least about 90%, undifferentiated, stem cells.

“Totipotent” refers to cells that are capable of differentiating into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells.

For the purposes of this disclosure, “neural precursor cell” or “neural precursor cell” mean a cell that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage as described below. Typically, they do not produce progeny of other embryonic germ layers when cultured by themselves in vitro, unless dedifferentiated or reprogrammed in some fashion. Neural precursor cells give rise to all types of neural cells: neurons, astrocytes and oligodendrocytes. Neural precursor cells, as used herein, describes a cell that is capable of undergoing greater than 20-30 cell divisions while maintaining the potency to generate neurons, astrocytes and oligodendrocytes. Preferably, said cells are capable of undergoing greater than 40, more preferably greater than 50, most preferably unlimited such cell divisions.

A “multipotent neural precursor cell population” is a cell population that has the capability to generate both progeny that are neuronal cells (such as neuronal precursors or mature neurons), and progeny that are glial cells (such as glial precursors, mature astrocytes, or mature oligodendrocytes), and sometimes other types of cells. The term does not require that individual cells within the population have the capability of forming both types of progeny, although individual cells that are multipotent neural precursors may be present.

Reference herein to a “population” of cells means two or more cells. A “homogeneous population” means a population comprising substantially only one cell type. A “cell type” may be cells of the same lineage or sub-type having substantially the same physiological status. Preferred homogeneous populations of the disclosure comprise substantially only early neuro-ectoderm-like Neural Precursor Cells (hNPCs). Reference to a “substantially pure homogeneous population of hNPCs” refers to a human neural precursor cell population in which a substantial number of the total population of the cells are of the same type and/or are in the same state of differentiation. Preferably, a “substantially pure homogeneous population” of neural cell precursor cells comprises a population of cells of which at least about 50% are of the same cell type, more preferably that at least about 75% are of the same cell type, even more preferably at least about 85% are of the same cell type, still even more preferably at least about 95% of the cells are the same type, and even more preferably at least about 97%, 98%, 99% or 100% are of the same cell type. In one embodiment, substantially homogeneous population of the disclosure is at least 99% of the same cell type. In another preferred embodiment, the preferred substantially homogeneous population of the disclosure is 100% of the same cell type. The substantially pure homogeneous population of hNPCs are generally obtained after about 10-12 days following the methods as disclosed herein.

Cancer stem cells are defined and functionally characterized as a small subset of cells from a tumor that can grow indefinitely in vitro under appropriate conditions (ability for self-renewal), are able to form tumors in vivo using only a small number of cells. Other common approaches to characterize CSCs involve morphology and examination of cell surface markers, transcriptional profile, and drug response. Furthermore, as used herein, the term “cancer stem cells” refers to cells that have the capacity to regenerate tumors at high frequency after transplantation and that have the capacity to expand and differentiate to all lineages of the tumor. It has long been known that a relatively small proportion of cells within differentiated tumors have the capacity to regenerate tumors at high frequency after transplantation (e.g., U.S. Pat. No. 6,984,522). This is indicative of rare populations of transformed cells that have the capacity to expand and differentiate to all lineages of the tumor. There is a need in the art for a natural model system in which to study the development of and signaling within a cancer stem cell population. Markers associated with cancer stem cells include, CD133, ALDH, VLA-2, .beta.-catenin, VLA-2, CD166, CD201, IGFR, EGF1R, Tweak (TNF-like weak inducer of apoptosis), EphB2, EphB3, human Sca-1 (BIG1), CD34, ESA, .beta.1 integrin (CD29), CD90, CD150, and CXCR4, IGF1-R, GPR49, CD166, and/or CD201.

The term “activity” as used herein refers to protein biological or chemical function.

The section headings used herein are for organizational purposes only and are not to be construed as limiting the subject matter described.

Several epigenetic markers have been used for qualitative immunofluorescence detection, yet quantitative analysis of epigenetic marker at a single cell level has not been undertaken. As disclosed herein, every cell type has unique epigenetic state, and such epigenetic state and/or epigenetic changes can be quantitatively detected and analyzed in a single cell level using a multiparametric image analysis: Microscopic Imaging of Epigenetic Landscape (MIEL). As epigenetic marks are present in every mammalian cell, it is contemplated that MIEL can be used to determine and to quantify heterogeneity within populations of cells, changes associated with pathology (e.g., epigenetic genetic markers known to be associated with beta amyloid accumulation in Alzheimer's disease, etc.). Further, as MIEL does not require absolute signal intensity of a given epigenetic marker, and unique pattern recognition algorithms are at work circumventing the inevitable intensity variations, analysis accuracy can be substantially increased compared to the other traditional epigenetic detection and analysis methods.

Aspects disclosed herein provide a method of determining an epigenetic signature of a primary cell by detecting a nuclear staining pattern and determining the epigenetic signature of the primary cell based on the nuclear staining pattern. As used herein, the primary cell can be any eukaryotic cells, any mammalian cells, or any types of human cells or cells derived from human cells. Thus, the primary cells can be obtained from any suitable source for obtaining cells, for example, fresh or frozen biopsy tissue (e.g., cancer tissue, adipose tissue, muscle, organ, etc.) of a subject (e.g., a mammal, a human, etc.), bodily fluid of the subject (e.g., blood, serum, a cerebrospinal fluid, etc.), fetal tissue, bone marrow, cord blood, etc.

In addition, the primary cells can be any types of nucleated or unnucleated cells, for example, hepatocytes, splenocytes, fibroblasts, embryonic stem cells, adult stem cells (e.g., induced pluripotent stem cells, etc.), pluripotent stem cells, neurons, astrocytes, cartilage cells, adipose cell, bone cells (e.g., osteoblast, osteocytes, etc.), platelets, immune cells (e.g., peripheral blood mononuclear cell (PBMC), T cells, B cells, NK cells, etc.), smooth muscle cells, or any other types of cells. In some instances, the primary cells can be diseased cells (e.g., infected cells, tumor cells, glioblastoma cells, etc.). In some instances, the primary cells can be acutely dissociated and cultured cells from the tissue (e.g., less than 48 hours, less than 24 hours, less than 12 hours, etc.). In some instances, the primary cells can be cells of an established cell lines.

Such obtained primary cells can be further processed to obtain cell-specific epigenetic patterns. In some instances, the cell-specific epigenetic pattern includes nuclear staining pattern, which can be detected using DNA dye, detection of a DNA modification, and/or detection of a histone modification. For example, gross chromatic structure can be detected and analyzed by direct staining of DNA with DNA non-intercalating dyes such as DAPI and by indirect immunofluorescence staining using antibodies against heterochromatin-binding proteins such as heterochromatin protein 1 (HP1). In other example, some DNA modifications (e.g., extrachromosomal DNA, double minute chromosome, DNA amplification, etc.) can be detected using DNA-fluorescence in situ hybridization (FISH) and/or electron spectroscopic imaging (ESI). Alternatively and/or additionally, changes in the nuclear organization and epigenetic landscape can be detected using genome-wide assays (e.g., ChIP-chip; ChIP-seq, etc.).

In certain instances, the cell-specific epigenetic pattern can be accurately assessed by detecting histone modifications (e.g., methylation, acetylation, phosphorylation, ubiquitination, sumoylation, etc.), which can affect gene expression by altering chromatic structures or recruiting histone modifiers. Thus, in some instances, gene expressions are detected in the nucleus of the cells, and/or subnuclear locations. For example, the several histone modifications may include H3K4me3, H3K4me1, H3K27me3, H3K27ac, H3K9me3, H3K9ac, H4K12ac, H4K12me3, and/or H3K18ac, which can be associated with various cellular states. For example, H3K9ac or H3K4me3 are generally associated with actively transcribing euchromatin, while H3K9me3 and H4K20me3 are in general associated with silenced heterochromatin. In another example, H3K27me3 is associated with facultative heterochromatin. Thus, it is contemplated that different types of histone modifications or their combinations can be used for determination of cell types, cell fates, cell status, cell states, etc.

Any suitable methods to obtain the nuclear staining pattern using above markers are contemplated. In some instances, the nuclear staining pattern can be obtained by labeling such markers with marker-specific antibodies that are further labeled with suitable fluorochrome-conjugated secondary antibodies. Fluorescent cell images are then detected and/or obtained using fluorescence microscopy, and such images can be analyzed to obtain cell features including nuclear morphology, fluorescence intensity inter-channel co-localization, and texture/or (bumpiness, gradients, etc.) features (e.g., Image moments, Haralick texture features, Threshold Adjacency Statistics, Gabor related features, radial features, etc.).

The cell features extracted from the fluorescent cell images can be further analyzed using subcellular feature analysis, a machine learning protocol, or a combination thereof, to obtain an epigenetic or multiparametric signature of the cell. For example, for machine learning, each of the extracted features is represented using a vector (center of distribution vectors), in which every element represents the average value of all cells in that population for a particular feature. Then some subsets are identified to use for training (control) and testing (test). The training sets are used to train a classifier using machine learning algorithms that best separates the controls. To prevent over training the test set and training sets are mutually exclusive. This process is repeated until a subset of features is identified with high predictive value in many classifiers. This subset of features is used in a final round of machine learning to generate an optimal classification method.

In some instances, different machine learning protocol or classifier (e.g., support vector machine, support vector regression, quadratic discriminant analysis, etc.) can be used based on the type of analysis and/or samples. For example, to visualize similarity between multiple cell populations, the multivariate centroids for each cell population and the Euclidean distance between all populations can be calculated. In another example, to visualize similarity between multiple cells in a similar group of cells (e.g., same or similar type of cells from different individual, different biological entity, different organs, etc.), the multivariate centroids for each cell and the Euclidean distance between all cells can be calculated. In another example, quadratic discriminant analysis can be used where the epigenetic or multiparametric signature of the primary cell is used to determine a cell population of the primary cell from multiple cell populations. In still another example, support vector machine can be used where the epigenetic signature of the primary cell is used to determine a character of the primary cell in a single cell population. In some instances, the epigenetic or multiparametric signature are quantified to be represented as MIEL “values”. In certain instances, the variation of MIEL values (e.g., obtained or analyzed from Haralick features or Shannon entropy of the cells) distributed around the centroid may provide an estimation of variance for a given biological entity. Such variance can be used as an informative measurement of epigenome homeostasis, or “epigenostasis”.

In some instances, the trained data can be further processed by principal component analysis. Within the context of the present disclosure, principal component analysis is a mathematical technique that transforms a number of correlated variables into a reduced number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables. Principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix. Other methods of analysis may be used, including a learning and pattern recognition analyzer comprises at least one algorithm selected from the group consisting of artificial neural network algorithms, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART), partial least squares (PLS), multiple linear regression (MLR), principal component regression (PCR), discriminant function analysis (DFA), linear discriminant analysis (LDA), cluster analysis, and nearest neighbor.

In one of the methods of principal component analysis, multidimensional scaling (MDS) can be used to reduce the multidimensional data (e.g., 3-dimensional topological distribution) into 2D projection. In MDS, the Euclidean distance between all vectors are calculated to assemble a dissimilarity matrix (size N×N, where N is the number of populations being compared). Then, the N×N matrix was reduced to a N×2 matrix with MDS using the Excel add-on program Xlstat (Base, v19.06), and displayed as a 2D scatter plot.

Several suitable measurements, calculations, and analysis scores may serve as miBioAge values. One important example of miBioAge quantification is an entropy, for instance a Shannon entropy, which is one of the features among several Haralick texture features computed for each image based on a pattern of epigenetic marks. Thus, MIEL value or MILE-CLOCK value includes a quantified value of computed entropy, for instance a Shannon entropy. Various miBioAge values from individual cells are distributed around the center (centroid) of a given biological entity (cell population, a subset of tissue, organ, organism). Such distribution provides an estimation of variance for a given biological entity (cell population, tissue, organ, organism). Variance of miBioAge values (e.g., variance of Shannon entropy) represents a valuable and informative measurement of epigenome homeostasis, or “epigenostasis.” Other quantifications that may serve as miBioAge values include nuclear morphology, fluorescence intensity inter-channel co-localization, texture (bumpiness, gradients, etc.) features (e.g., Image moments, Haralick texture features, Threshold Adjacency Statistics, radial features, etc.). miBioAge values can be also obtained from quantitative analysis of a subcellular feature analysis, a machine learning protocol, principal component analysis, or a combination thereof using previously described miBioAge values.

In some instances, such method of determining epigenetic or multiparametric signature can be used to further determine or evaluate various cell characteristics of the primary cells. For example, where the primary cell is a pluripotent stem cell or a progenitor cell, the epigenetic signature can be further used to determine a cell fate of the primary cell (e.g., whether the pluripotent cell will be differentiated into a fibroblast or a neuroblast, etc.), or whether two distinct primary cells will be differentiated into a same type of cell or two different types of cell. Alternatively and/or additionally, where the primary cell is a tumor propagating cell, the epigenetic signature can be further used to determine a differentiation signature of such tumor propagating cell (e.g., whether the tumor propagating cell is differentiating into a cell type that are different from other tumor cells in the tumor mass to obtain resistance, etc.). Alternatively and/or additionally, the epigenetic signature can be further used to determine an epigenetic efficacy of a drug. In this example, the primary cell can be treated with a drug (e.g., in various doses, in various schedules, etc.), and epigenetic or multiparametric signature of the primary cell before and after the treatment, or during the treatment can be determined. It is contemplated that an epigenetically active drug may change at least one or more epigenetic or multiparametric signature of the primary cell upon or during the drug treatment.

Additionally, such drug treated primary cells can be further monitored for any presence of senescence due to drug treatment by detecting the nuclear staining pattern and determining the epigenetic signature of the primary cell before and after treatment and/or periodically during and after the treatment. It is contemplated that an epigenetically active drug that affects cell senescence can result in changes in one or more epigenetic signatures (e.g., changes in H3K9me2/3, H3K27me3, and H4K20me3, etc.) in the cell. In some instances, any environmental substance(s) (e.g., light (infrared light, visible light, UV, etc.), a sound (e.g., noise, ultrasound, sound in specific frequencies, etc.), a mechanical vibration, an electrical shock, or a pressure, etc.) can be used to apply the primary cell to induce changes in epigenetic signature(s) such that one can determine which environmental substance(s) negatively or positively affect the cell health or survival, or which environmental substance(s) may affect the cell fate, differentiation, and/or senescence. Further, drug treatment and the environmental substance treatment can be combined to determine which environmental substance can adversely, neutrally, or beneficially affect the efficacy of epigenetic drug, or conversely, which epigenetic drug can adversely or beneficially affect the effect of environmental substance to the primary cell.

In another aspect of the present disclosure provides that the image-based multiparametric analysis can be used to screen the drug efficacies, and thus improve the drug discovery process. In certain instances, two groups of primary cells (preferably same types of cells maintained or obtained in a substantially same condition and/or from same subject) are treated with two drugs (preferably two epigenetically active drugs), or two different concentrations of a single drug, and the nuclear staining patterns of one or more epigenetic markers (e.g., H3K27me3, H3K9me3, H3K27ac, and/or H3K4me1) of two groups of cells. Based on the nuclear staining patterns, the epigenetic or multiparametric signatures of the drug treated primary cells can be obtained and compared. For example, the magnitude and/or direction of the changes in the epigenetic or multiparametric signatures between the drug treated primary cells (treated with drug A versus treated with drug B, or treated with concentration C of drug A versus concentration D of drug A) and/or each compared to the epigenetic or multiparametric signatures before the treatment can be evaluated to determine the ranking of the drugs to the primary cells. In some instances, the ranking can be based on which drug is more effective to the primary cells (e.g., effective to treat certain condition of the cells, effective to reverse cellular aging of the cells, effective to induce differentiation, effective to prevent aging or slow down aging process, effective to reduce or prevent tumorigenesis, etc.) or has more adverse effect (e.g., effective to induce pathological changes in the cells, effective to induce or accelerate cellular aging, effective to induce tumorigenesis, effective to induce mutagenesis, etc.) to the primary cells (drug activity ranking). In other instances, the ranking can be based on which concentration of the drug is more effective to the primary cells or has more adverse effect to the primary cells (drug concentration effect ranking).

In some instances, the image-based multiparametric analysis can be further used to evaluate, determine, or predict synergistic or antagonistic effect of combination of drugs. Two groups of primary cells (e.g., same types of cells maintained or obtained in a substantially same condition and/or from same subject) are treated with two epigenetically active drugs A or B can be treated with drug C, either before, concurrently, after treatment of drugs A or B. Alternatively, Two groups of primary cells (e.g., same types of cells maintained or obtained in a substantially same condition and/or from same subject) are treated with first epigenetically active drug A can be treated with drug B or drug C, either before, concurrently, after treatment of drugs A. The magnitude and/or direction of the changes in the epigenetic or multiparametric signatures of between the drug treated primary cells and/or each compared to the epigenetic or multiparametric signatures before the treatment can be evaluated to determine whether the combination of drugs add synergistic effect to the single drug treatment, or reduce the effect of the single drug treatment (antagonistic effect), or provide adverse effect to the cell (e.g., affect the cell health regardless of the efficacy of the single drug, etc.).

In another aspect of the present disclosure provides that the image-based multiparametric analysis can be used to determine biological ages of two primary cells in a single cell level by detecting expression patterns of epigenetic marks of the primary cells, determining multiparametric signatures of the primary cells, and comparing them together by plotting the multiparametric signatures against the primary cell's chronological ages. As used herein, cell's “biological age” refers the cell's physiological age, which may or may not match with cell's own chronological age. For example, a cell may have a biological age (e.g., 1 month physiological old skin cell) younger than its actual chronological age (actual 3 month old since the cell was actually generated or differentiated to the specific cell type-skin cell). In such example, the biologically younger cell may show different physiological activity or different physiological activity level than the cells in the same chronological age that are biologically older.

In some instances, two primary cells are obtained from the same subject (e.g., same person, same mammal, same tissue, same organ, same cell population, etc.). In other instances, the two primary cells are obtained from different subjects (e.g. different people, different mammals, different tissues, different organs, different cell populations, etc.). The two primary cells may be of different cell types or the same cell type. The cells may be of any type, including hepatocyte, a fibroblast, a peripheral blood mononuclear cell, or an immune cell. In some instances, the cells are of different ages (e.g., chronological age), or the same age (e.g., chronological age). In some instances, the cells are in the same, or substantially similar stage of development and/or differentiation. In some instances, the cells are in different development and/or differentiation.

Physiological activity of the cell can be detected by determining variability in epigenetic marker expression or modification patterns that may change upon biological aging of the cells. In certain instances, Epigenetic markers are molecules or signals that correspond to epigenetic marks. Epigenetic marks are those that relay some physical and chemical attribute of an epigenome including histone modification, chromatin shape, DNA modification, non-coding RNA, one or more genetically encoded epigenetic probes, or other known epigenetic attribute. Such marks may include histone modifications, including H3K79me, H3K36me, H3K27me3, H3K27ac, H3S10p, H3K9me3, H3K9ac, H3K4me1, H2AT120p, H2AK119ub, H2BK120me, H4R3me, H4R3cit, H4K5ac, H4K8ac, H2AXK119ub, and H2AXS139p. In some instances, a single epigenetic marker can be used to determine the biological age of a cell. In some instances, a combination of at least two, at least three, at least four, at least five epigenetic markers can be used to determine the biological age of a cell.

In some instances, changes in expression or modification patterns of such markers can be detected by labeling the epigenetic markers with any visualization tags (e.g., metal labeling, fluorescent labeling, radio-isotope labeling, etc.). In such instances, detecting expression patterns of epigenetic marks can be achieved by any visualization tools detecting such tags, including, but not limited to, immunofluorescence, scanning electron microscopy, atomic force microscopy, or other suitable microscopy methods, detecting epigenetic markers. In some instances, the changes in expression or modification patterns of such markers can be detected by detecting one or more genetically encoded epigenetic probes. In some instances, detecting expression patterns of the plurality of epigenetic marks in the cells comprises capturing a series of images of the first plurality of primary cells and the second plurality of primary cells over a period of time. In some instances, capturing a series of images of the plurality of primary cells over a period of time comprises capturing at least one image of first plurality of primary cells and the second plurality of primary cells before, during, and after mitosis.

Based on the nuclear staining patterns, the epigenetic or multiparametric signatures of the two primary cells can be determined. In some instances, the epigenetic or multiparametric signatures of a biologically old cell differ from the epigenetic signature of a biologically young cell based on at least one nuclear staining pattern when the biological ages of cells differ at least 1 day, at least 2 days, at least 3 days, at least 4 days, at least 5 days, at least 6 days, at least 1 week (e.g., 1 week old cell v. 2 week old cell, etc.), at least 2 weeks, at least 4 weeks, at least 2 months, at least 3 months, etc. Thus, biological ages of two primary cells can be determined based on the epigenetic signatures of two primary cells.

In some instances the staining or expression patterns used to determine the epigenetic signatures of primary cells are representative of chromatin shape, DNA modification, histone modification, post-translational protein modification, RNA expression or specific sequences, DNA copy numbers or specific sequences, protein expression, metabolite or other small molecular concentration, ion concentrations, detection of a nuclear staining pattern, detection of one or more genetically encoded epigenetic probes, etc., or any combinations thereof such histone modifications can include acetylation, methylation, SUMOylation, ubiquitinylation, nitrosylation, phosphorylation, adenylation, or other known post-translational modification. Specific histone modifications may include H3K4me3, H3K4me1, H3K27me3, H3K27ac, H3K9me3, H3K9ac, H4K12ac, H4K12me3, H3K18ac, and any combinations thereof. In some instances, the patterns may be detected and classified by intensity, variation, and/or distribution.

In some instances, the multiparametric signature comprises a texture-associated feature or multiple features. Texture associated features are derived from co-occurrence matrices of intensity or grayscale values of images or various dimensions of color. Such features may include Haralick texture features, threshold adjacency statistics, Gabor related features, radial features, or any combinations thereof. These features can be further analyzed using subcellular feature analysis, machine feature extraction algorithms, machine learning protocols, or a combination thereof. The machines learning protocols may include machine learning algorithms including support vector machine, linear regression, quadratic discrimination analysis, neural network, or a combination thereof. The outputs of these analysis methods of protocols can be modified by reducing a data dimensionality of the expression patterns or multiparametric signatures.

The output of these analysis methods or protocols can be shown as multiparametric scatter plots, 3-dimensional topological distributions, bar graphs, or centroids. It is contemplated that centroids reflect the geometric center of a collected number of values pertaining from the same group of cells, tissues, individuals, biological entity, or any other group or single source suitable for comparison between the populations of primary cells, determining relationships between the primary cells, or contrasting the populations of primary cells from each other. Relationships, comparisons, and/or contrasts between the populations of primary cells may be expressed as calculated Euclidian distances. Furthermore, relationships, comparisons, and contrasts can be made between different individuals, organs, and tissues where the pluralities of primary cells were isolated from different individuals, organs, and tissues. Relationships, comparisons, and contrasts can be made between the centroids of the pluralities of the primary cells by comparing, contrasting, and finding relationships between their multiparametric signatures.

Comparing, contrasting, or determining relationships of the multiparametric signatures of the plurality of primary cells against other pluralities of primary cells can entail plotting their respective MIEL-values, multiparametric signatures, centroids, or other output described above, against chronological age. In certain instances, chronological age refers to the age of the animal or source that the cells were isolated from, the time that the cells were cultured ex vivo, or the time since the cells were removed from a parental animal in utero. Comparing the multiparametric signatures of the plurality of primary cells against other pluralities of primary cells can entail plotting their respective MIEL-values, multiparametric signatures, centroids, or other output described above against a histogram of output range, multidimensional scaling factors, or other suitable variables. For example, the multiparametric signatures are compared by determining a first centroid of a first element of the multiparametric signature of the first plurality of primary cells and a second centroid of the first element of the multiparametric signature of the second plurality of primary cells. In another example, the multiparametric signatures are compared by determining a plurality of centroids of each of the multiparametric signatures of the first plurality of primary cells and the second plurality of primary cells. In such instances, a multivariant centroid of the plurality of cells can be calculated, and in some instances, the multivariant centroids can be plotted relative to the chronological ages of the plurality of cells.

From comparing, contrasting, or determining relationships between the primary cells using their multiparametric signatures, centroids, and other outputs outlined above, a chronological or biological age may be determined. The chronological age can be determined by determining the biological age of the plurality of primary cells and the biological age can be determined by determining the chronological age of the plurality of primary cells. Alternatively, biological age can be determined independently from the chronological age of the cell. The biological age may be expressed or calculated as a derivation from a known chronological age, and the chronological age may be expressed or calculated from a known biological age. Biological or chronological ages may be expressed as seconds, minutes, hours, day, weeks, months, years, cell culture passage amounts, etc.

In some instances, the primary cells may be classified into groups determined by their chronological age, biological age, multiparametric signatures, and/or epigenetic marks. Such groups may include healthy, sick, diseased, older, younger, or other general descriptors of cell health, physiological status, and/or age. It is further contemplated that cells discriminated by biological age may be classified into groups such as biologically young cells or biologically old cells. Multiparametric signatures and epigenetic marks may be associated with aging.

It is further contemplated that such changes or differences in epigenetic signatures between biologically young and biologically old cells can be used to identify any drugs, small molecules, and/or environmental substances that may affect cell aging or senescence. In certain embodiment, a primary cell can be contacted or treated with a drug, a small molecule, and/or an environmental substance for at least 1 min, 5 min, 10 min, 30 min, 1 hour, 2 hours, 3 hours, 6 hours, 12 hours, 24 hours, 2 days, 3 days, 7 days, or more, and nuclear staining patterns of one or more epigenetic markers (e.g., H3K27me3, H3K9me3, H3K27ac, and/or H3K4me1, etc.) of the primary cell is detected. Based on the nuclear staining patterns, the epigenetic or parametric signature of the primary cell can be determined and compared with the epigenetic or multiparametric signature of another primary cell that is at least 1 week, 2 weeks, 4 weeks, 2 months, 3 months, 6 months, 12 months old cell. It is contemplated that the primary cell that is contacted or treated with a drug, a small molecule, and/or an environmental substance that facilitates cell aging may show the nuclear staining patterns and the epigenetic signature similar to the biologically older primary cells. In some instances, the treatment to the primary cell induces an epigenetic change without exerting cytotoxicity proportional to the epigenetic change to the primary cell. In some instances, the treatment to the primary cell induces an epigenetic change without exerting cytotoxicity nonlinearly to the epigenetic change to the primary cell.

In some instances, determining the effect of the treatment on the primary cell based at least in part on the comparing of the multiparametric signatures comprises determining the effect on the biological age of the primary cell and/or determining the effect on cell division.

Alternatively and/or additionally, such changes or differences in epigenetic or multiparametric signatures between biologically young and biologically old cells can be used to identify any drugs, small molecules, and/or environmental substances that may affect differentially to cells in different chronological ages. In some instances, two primary cells are contacted, or “treated”, with a drug, a small molecule, nucleic acid, protein or fragment thereof, antibody, and/or an environmental substance or condition for at least 1 min, 5 min, 10 min, 30 min, at least 1 hour, 2 hours, 3 hours, 6 hours, 12 hours, 24 hours, 2 days, 3 days, 7 days, or more, and nuclear staining patterns of one or more epigenetic markers (e.g., H3K27me3, H3K9me3, H3K27ac, and/or H3K4me1) of the primary cells. Based on the nuclear staining patterns, the epigenetic or multiparametric signatures of the primary cells can be determined and compared with each other. It is contemplated that a drug, a small molecule, antibody, protein, and/or an environmental substance (e.g., having age-specific effect) would affect the epigenetic signature of a primary cell in a specific age to change at least 10%, at least 20%, at least 30%, at least 40%, at least 50%, at least 100% more than primary cells in different ages when compared with the epigenetic or multiparametric signature before contact with such drug, small molecule, or environmental substance, thus determining the effect of a treatment.

In some instances, the primary cells can be as described herein, such as hepatocytes, fibroblasts, PBMCs, lymphocytes, stem cells, progenitor cells, fetal cells, embryonic stem cells, neurons, astrocytes, smooth muscle cells, cancer cells, induced pluripotent stem cells, or neural stem cells.

In some instances, the treatment may comprise various types of drugs, a small molecules, nucleic acids, proteins or fragments thereof, antibodies, and/or an environmental substances or conditions. Drugs may consist of FDA-approved drugs, drug candidates, experimental drugs, drug library compounds, or pharmaceutically active drugs or fragments thereof. Small molecules may consist of synthetic or organic compounds less than 1000 g/mol molecular weight. Nucleic acids may include DNA, RNA, siRNA, microRNA, long-noncoding RNA, ssDNA, ssRNA, dsDNA, multiplexed RNA, synthetically modified nucleic acids, naturally modified nucleic acids, nucleic acids with non-canonical nucleotides. Proteins may include short peptide less than 100 amino acids in length, larger peptides longer than 100 amino acids in length, folded proteins, unfolded proteins, post-translationally modified protein, and synthetic proteins. Antibodies include IgG antibodies, IgM antibodies, IgA antibodies, IgD antibodies, IgE antibodies, IgY antibodies, IgW antibodies, Fc region segments, Fab region segments, and antibody fragments. Environmental substances and conditions include environmental toxins, light, sound, vibrations, electrical shocks, pressure, humidity, particle radiation, electromagnetic radiation, or known chemical pollutants. It is contemplated that the treatment may be an inducer of cell rejuvenation, cell senescence, or epitoxicity.

In some instances, the treatment may take place in an incubator, multi-well plate system, cell culture dish, synthetic container, or vessel. The treatment may be applied or dispensed in order to contact the cells manually, or automatically by a robotic liquid handler system amenable for automated high throughput screening. In some instances, dozens or thousands of multi-well plate system, e.g., 96 wells, 384 wells, 1536 wells, 3456 wells, 9600 wells, or other commercially available size, are seeded with primary cells for high throughput screening. The above instances may also use induced pluripotent cells, cancer cell lines, or other cell lines instead of primary cells. Such cells may also be seeded in multi-well plates for high throughput screening. Multi-well plates may be treated for a variable amount of time as described previously with the various treatments and then read by a suitable high content imager or immunofluorescent microscope capable of reading multiple channels and plates. Such imagers or microscopes may be coupled to a computer capable of processing and analyzing the multiparametric signatures and epigenetic marks described herein.

It is contemplated that the effects of treatment on the multiparametric signatures and epigenetic marks may be associated with aging, young cells, old cells, cell sensitivity, cell metabolism, cell signaling, cell cycle, cell health, mitochondrial health, telomere length, or abundance of prion-like protein concentration. Prion-like protein concentration may include the concentration of mis-folded or unfolded proteins implicated in prion-associated disease such as Alzheimer's, Creutzfeldt-Jakob disease, Amyotrophic lateral sclerosis, Parkinson's disease, etc.

In some instances multiparametric signature and epigenetic mark changes can be detected in treated primary cells that will shift closer or become similar as those as an untreated cell that is a younger cell, an older cell, a different cell type, a different tissue type, or a different biological entity.

A treatment may be identified as anti-aging or rejuvenating. A treatment can be identified as anti-aging or rejuvenating when the multiparametric signature or epigenetic mark of a treated cell is related to or similar to a younger or healthier untreated cell. In some cases, the treatment does not exert cytotoxicity to the cells while causing a shift in the multiparametric signature or epigenetic mark of the cell. A treatment can be identified as pro-aging or cell senescence inducing. A treatment can be identified as pro-aging or cell senescence inducing when the multiparametric signature or epigenetic mark of a treated cell is related to or similar to an older or unhealthy untreated cell. A treatment effect to the multiparametric signature or epigenetic mark of the cell can be related to a tissue, organ, or biological entity.

In some instances, the multiparametric signature or epigenetic mark change is determined by comparing the epigenetic change of the treated primary cell to an epigenetic change of a primary cell treated with a known epigenetically active compound. Known epigenetically active compounds may include HDAC inhibitors, EZH2 inhibitors, AURORA kinase inhibitors, SIRT inhibitors, or any combinations thereof.

In some instances, multiple treatments are applied to the same cell. A cell population, including the primary cell, may be taken from the same tissue, organ, or biological entity and different groups of cells from the same population may be treated with any combination of treatments to determine the effect of the different treatments on the primary cell. Such methods may be used with a plurality of cell populations to determine the effect of the treatments on various different primary cells and cell populations.

Another aspect of the disclosure includes methods of detecting aging or maximum individual lifespan of a biological entity. In these methods, expression patterns of a plurality of epigenetic marks in a plurality of primary cells of a biological entity are detected and multiparametric signatures of the plurality of primary cells are determined based on the expression patterns of the plurality of epigenetic marks. In some instances, average value of coefficient of variance of multiparametric signatures of the plurality of primary cells is determine. In some instances, the aging or maximum individual lifespan of the biological entity is determined based at least in part on the average value of coefficient of variance of multiparametric signatures.

Another aspect of the disclosure includes method of predicting a residual lifespan of a biological entity. In these methods, expression patterns of a plurality of epigenetic marks in a first plurality of primary cells and in a second plurality of primary cells of a biological entity are detected, where the first and second plurality of primary cells are associated with different chronological ages. Then, multiparametric signatures of the first and second plurality of primary cells are determined based on the expression patterns of the plurality of epigenetic marks. In some instances, average values of coefficient of variance of multiparametric signatures of the first and second plurality of primary cells are determined, and the residual lifespan of the biological entity based at least in part on the changes of the average values of coefficient of variance of multiparametric signatures.

In some instances, the chronological ages of the first and second plurality of primary cells are at least 60 days, 90 days, 120 days, 180 days, 1 year, 2 years, 3 years different from each other.

In certain instances, the cell-specific epigenetic pattern can be accurately assessed by detecting histone modifications (e.g., methylation, acetylation, phosphorylation, ubiquitination, sumoylation, etc.), which can affect gene expression by altering chromatic structures or recruiting histone modifiers. Thus, in some instances, gene expressions are detected in the nucleus of the cells, and/or subnuclear locations. For example, the several histone modifications may include H3K4me3, H3K4me1, H3K27me3, H3K27ac, H3K9me3, H3K9ac, H4K12ac, H4K12me3, and/or H3K18ac, which can be associated with various cellular states. For example, H3K9ac or H3K4me3 are generally associated with actively transcribing euchromatin, while H3K9me3 and H4K20me3 are in general associated with silenced heterochromatin. In another example, H3K27me3 is associated with facultative heterochromatin. Thus, it is contemplated that different types of histone modifications or their combinations can be used for determination of cell types, cell fates, cell status, cell states, etc.

In some instances, expression patterns can be detected by capturing a series of images of the cells over a period of time. In some instances, capturing a series of images of the cells over a period of time includes capturing at least one image of the cells before, during, and after mitosis.

In some instances, the biological entity is a tissue, an organ, or an organism. In some instances, the biological entity is a diseased tissue, a diseased organ, or a diseased organism. In some instances, the primary cell is a hepatocyte, a fibroblast, a peripheral blood mononuclear cell, or an immune cell. In some instances, the primary cell is a cell treated with a drug, a chemical agent, or has been exposed to an environmental toxin, radiation, light, or temperature.

Any suitable methods to obtain the nuclear staining pattern using above markers are contemplated. In some instances, the nuclear staining pattern can be obtained by labeling such markers with marker-specific antibodies that are further labeled with suitable fluorochrome-conjugated secondary antibodies. Fluorescent cell images are then detected and/or obtained using fluorescence microscopy, and such images can be analyzed to obtain cell features including nuclear morphology, fluorescence intensity inter-channel co-localization, and texture/or (bumpiness, gradients, etc.) features (e.g., Image moments, Haralick texture features, Threshold Adjacency Statistics, Gabor related features, radial features, etc.). In some instances, a nuclear staining pattern and/or one or more genetically encoded epigenetic probes can be detected from a fixed cells and/or from a live cells.

In some instances, the expression pattern comprises an expression intensity or distribution of at least one of the plurality of epigenetic marks.

The cell features extracted from the fluorescent cell images can be further analyzed using subcellular feature analysis, a machine learning protocol, or a combination thereof, to obtain an epigenetic or multiparametric signature of the cell. For example, for machine learning, each of the extracted features is represented using a vector (center of distribution vectors), in which every element represents the average value of all cells in that population for a particular feature. Then some subsets are identified to use for training (control) and testing (test). The training sets are used to train a classifier using machine learning algorithms that best separates the controls. To prevent over training the test set and training sets are mutually exclusive. This process is repeated until a subset of features is identified with high predictive value in many classifiers. This subset of features is used in a final round of machine learning to generate an optimal classification method.

In some instances, increased average value of coefficient of variance of multiparametric signatures indicates onset of aging of the biological entity. In some instances, increased average value of coefficient of variance of multiparametric signatures indicates increased biological age of the biological entity.

In some instances, the residual lifespan of the biological entity is predicted by a function of CV=c−a/(x−b), where x is the age axis (age), b is the average or individual maximum life span, and c and a are additional parameters that can be determined experimentally from the curve/equation fitting. CV is coefficient of variance (CV=σ/μ where σ=population standard deviation and μ=population mean). In some instances, the initial or current state of the organism such as aging is captured by parameter c and the degree or the speed of change at a given time period is captured by parameter a.

Another aspect of the disclosure includes methods of improving accuracy of imaging analysis of a primary cell at a single cell level, using co-occurrence of epigenetic marks (CINEMA). In some aspects, improvement of accuracy of imaging analysis of the primary cell is achieved by detecting expression patterns of a plurality of epigenetic marks in a primary cell, determining co-occurrence of the expression patterns of the plurality of epigenetic marks and chromatin density; and determining multiparametric signature of the primary cell based at least in part on the co-occurrence of the expression patterns. In some instances, detecting expression patterns of a plurality of epigenetic marks in a primary cell comprises detecting expression patterns of a plurality of epigenetic marks in a nucleus of the primary cell. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the multiparametric signature comprises a texture-associated feature. In some instances, the texture-associated feature comprises Haralick texture features, threshold adjacency statistics, radial features, Gabor related features, or other types of texture features and any combination thereof. In some instances, the co-occurrence comprises frequency or spatial distribution of the expression patterns that cooccur in the nucleus of the primary cell. In some instances, the co-occurrence comprises frequency and spatial distribution of the expression patterns that cooccur in the nucleus of the primary cell. In some instances, the methods described herein further comprise quantifying first and second pixel values of first and second epigenetic marks at a plurality of locations in the nucleus of the primary cell and first and second probabilities of density of the first and second epigenetic marks at the plurality of locations.

In some aspects, the present disclosure also provides methods of determining an effect of a treatment on a primary cell, comprising: applying the treatment to a primary cell to obtain a treated primary cell; detecting expression patterns of a plurality of epigenetic marks in the treated primary cell and an untreated primary cell; determining co-occurrence of the expression patterns of the plurality of epigenetic marks and chromatin density; and determining the effect of the treatment on the primary cell based at least in part on a difference of co-occurrence of the expression patterns between treated primary cell and the untreated primary cell. In some instances, detecting expression patterns of a plurality of epigenetic marks in the treated primary cell and an untreated primary cell comprises detecting expression patterns of a plurality of epigenetic marks in a nucleus of the treated primary cell and in a nucleus of the untreated primary cell. In some instances, detecting the expression pattern comprises detection of chromatin shape, detection of a DNA modification, detection of a histone modification, detection of a nuclear staining pattern, detection of one or more genetically encoded epigenetic probes, or a combination thereof. In some instances, the expression pattern comprises an expression intensity or distribution of at least one of the plurality of epigenetic marks. In some instances, the histone modification is selected from the group consisting of: H3K4me3, H3K4me1, H3K27me3, H3K27ac, H3K9me3, H3K9ac, H4K12ac, H4K12me3, H3K18ac, and any combinations thereof. In some instances, the treatment comprises an exposure to a small molecule, radiation, light, temperature, an environmental exposure, or a combination thereof. In some instances, the primary cell comprises a hepatocyte, a fibroblast, a peripheral blood mononuclear cell, a lymphocyte, a stem cell, a progenitor cell, a fetal cell, an embryonic stem cell (ESC), an induced pluripotent cell (iPSC), a neural stem/precursor cell, a neuron, an astrocyte, a smooth muscle cell, or a tumor cell. In some instances, co-occurrence of the expression patterns comprises frequency or spatial distribution of the expression patterns that cooccur in the nucleus of the primary cell. In some instances, the co-occurrence comprises frequency and spatial distribution of the expression patterns that cooccur in the nucleus of the primary cell.

In some instances, the methods described herein further comprise quantifying first and second pixel values of first and second epigenetic marks at a plurality of locations in the nucleus of the primary cell and first and second probabilities of density of the first and second epigenetic marks at the plurality of locations. In some instances, the difference of the co-occurrence comprises a difference in frequency or spatial distribution of the expression patterns. In some instances, determining the effect of the treatment on the primary cell based at least in part on the comparing of the multiparametric signatures comprises determining the effect on the biological age of the primary cell and/or determining the effect on cell division. In some instances, detecting expression patterns of a plurality of epigenetic marks in a plurality of primary cells of a biological entity comprises detecting expression patterns of a plurality of epigenetic marks in a nucleus of the plurality of the primary cells comprises capturing a series of images of the plurality of the primary cells over a period of time. In some instances, capturing a series of images of the plurality of the primary cells over a period of time comprises capturing at least one image of the plurality of the primary cells before, during, and after mitosis.

There is a dramatic improvement in resolution using co-occurrence of different epigenetic marks within the same nucleus compared to classical statistical features such as Haralick texture features or threshold adjacency statistics features. In some instances, the measurement of chromatin density is included in the analysis since DAPI (intercalating molecule reporting the DNA density) is always present in the images. The joint probability maps are used to quantitate the co-occurrence of different epigenetic marks as well as chromatin density using DAPI staining. In some instances, co-occurrence of different epigenetic marks and/or chromatin density means that one or more epigenetic marks and/or chromatin density are at least partially colocalized. In some instances, co-occurrence of different epigenetic marks and/or chromatin density means one or more epigenetic marks and/or chromatin density are localized within a subnucleolar location with or without colocalization. In some instances, co-occurrence of different epigenetic marks and/or chromatin density means one or more epigenetic marks and/or chromatin density are detected within the same nucleus. In some instances, co-occurrence of different epigenetic marks and/or chromatin density means one or more epigenetic marks and/or chromatin density are detected within a subcellular location of the cell.

The power of joint probability maps is tested using previously published dataset obtained by comparing 222 epigenetically active compounds split into 24 functional categories. Previously, epigenetic effect of ˜122 compounds covering 19 classes was detected. Previously, detecting all compounds was achieved in only 3 functional classes of compounds. Surprisingly, using joint probability maps approach all 24 functional categories were able to be detected and separated from each other with 100% accuracy.

It is contemplated that CINEMA can be applied to any biological question using image analysis of single cell nuclei. For example, it could be applied to analyze epigenetic signatures of aging and its perturbations. In this case comparison between all life stages and interventions/perturbations will result in the difference matrix that could be used to measure the distances to obtain the miBioAge quantitation as well as variance to obtain variance of chromatin and epigenetic landscape (VITA) score.

It is contemplated from documented example with univariate, bivariate, and trivariate analyses that increasing the number of channels to 5, 15, 20 channels by means of multiplexing/sequential imaging with either published academic protocols or commercial platform such as CODX.

In addition to conducting CINEMA in 2D version, it is contemplated that the same algorithms, including joint probability maps, could be developed in 3D using voxels to bin the fluorescence intensities distribution in the volume. The computational approach is quite similar to that developed for the computations in 2D.

For instance, joint probability maps accurately classify epigenetically active compounds. Cooccurrence analysis based on trivariate joint probability maps result 100% accuracy of separation of all 24 functional classes. CINEMA could be applied to various aspects of drug discovery, drug combinations, analyses of environmental compounds, cancer field—to discover drugs that differentiate cancer stem cells into non-tumorigenic cells. Multivariate (5-10-20-40 epigenetic marks) CINEMA analyses will be exponentially more powerful. Multivariate CINEMA analyses can be combined with classical statistical texture features (Haralick and TAS). Multivariate CINEMA analyses can be applied to 3D (voxels). e.g. joint probability maps algorithms formalism is agnostic to the spatial origin of the channel intensity measurements. Namely, pixels with X Y coordinates or voxels with X Y Z coordinates. Multivariate CINEMA analyses can also be applied to 3D (voxels) combined with classical statistical texture features (Haralick and TAS) in 3D.

CINEMA is a fundamental improvement over common statistical texture features. In some instances, CINAMA is used for analyses of epigenetic marks. It is contemplated that CINAMA can be used analyses of marks. It is contemplated that miBioAge and VITA, can be done with CINEMA (co-occurrence features). It is further contemplated that the methods disclosed herein including miBioAge, VITA and CINEMA can be done in two-dimensions (i.e., pixels) and in three-dimensions (i.e., voxels). It is contemplated that accuracy is increased with increased image resolution, and is dramatically improved with transition from 2D into 3D modality.

MIEL-live labeling of epigenetic marks in a live cell can be applied to mi-BioAge, VITA, and CINEMA. MIEL-live enables following each individual cell over time, without killing the cell. This is significantly different from conventional methods that “kill” the cells to acquire its signature, in which case it can only be done once. Thus, the power of MIEL-live is to uncover the change in all of the above along the life-span/trajectory of each single cell. It is further contemplated that MIEL-live labeling can be done in two-dimensions (i.e., pixels) and in three-dimensions (i.e., voxels).

The incidence of all aging-associated diseases (atherosclerosis, cardiovascular, cancer, arthritis, cataracts, osteoporosis, type 2 diabetes, hypertension, Alzheimer's) increases exponentially with age. Several studies provided evidence for the increase in chromatin and gene expression heterogeneity with age.

As disclosed herein, a novel computational approach is developed to relating multidimensional datasets from various experimental models. In some instances, in addition to computing the values of individual cell nucelli in multidimensional space of statistical texture features, coefficient of variance (CV) is used, defined as the ratio of the standard deviation a sigma to the mean p, namely. CV for each field of view, well, technical replicate, and biological replicate such organs tissues and cells from individual mice could be computed based on individual nuclei and the results are stable using 200-2000 nuclei per experimental replica (e.g., individual wells). Organs, tissues, and cells from individual mice often have distinct CV values. This means that quantitatively CV can be informative values serving as a quantitative measurement of individual organ, tissue, or cell type from individual animals. CVs computed using single cell signatures from of all organs and tissues examined invariably increase with age. Namely, CV from the samples of older age are greater than CV from the same type of organs, tissues, and cells from younger animals. This is a universal phenomenon that is likely to be extended to all organs and tissues, including for example peripheral blood, liver, brain, muscle and heart, in all animals including humans.

Further, hyperbolic type function: CV=c−a/(x−b) where x is the age, provides the best fit for the increase of CV with age in peripheral blood mononuclear cells (PBMC). Because such function has a true singularity (x=b) one can compute the theoretical maximum lifespan—the point on x axis (age axis) when the CV increase to infinity (x=b). Using this method, the maximum lifespan of C57BL/6 mice was predicted at ˜47 months. This prediction fits well with the limit of observed lifespans, ˜36 months for the highly inbred C57BL6 mice (over 90% mortality linked to cancer and other age-associated diseases) and ˜51 months for the wild type mice.

Data presented herein show a trend for the increase of VITA score after DOX treatment and a decrease of VITA score after CR treatment suggesting that VITA may serve as an indicator of functional/biological aging.

Further, PBMC is split using CD3 marker into subpopulations of CD3+ compartment representing all T cells and CD3− compartment representing a mixture of B cells and monocytes. Data presented herein show that each cell population provides different prediction with respect to maximum lifespan. This can be applied to each cell population within the organism and may reflect different resilience or longevity of different cell populations.

Further, CV could be computed based on quantitation of different epigenetic makes or chromatin density. For example, DAPI, present in all staining combinations to provide nuclear mask, provides variance of chromatin organization. Alternatively, specific epigenetic marks (e.g. H3K27 ac or H3K27me3) will provide variance with respect to distribution of these marks across the chromatin manifold. The behavior of CV computed for different cell types and using different epigenetic marks is different. For example, variance (CV) of H3K4me1 mark increases hyperbolically with age in both CD3+ and CD3− cell populations, whereas the variance of chromatin density (using DAPI) change much less if at all with age.

Finally, the 2D version of analyses using pixel-based statistics. The 3D version of the same type of analyses using voxel-based statistics will be dramatically more powerful. The computational algorithms to compute texture features in 3D such as Haralick and TAS are similar to that employed for 2D and will include the knowledge of 3D neighborhood as opposed to the current 2D neighborhood computations.

Epigenetic heterogeneity, here referred as variance of chromatin and epigenetic landscape (VITA) computed at single cell level is often distinct in different biological samples (e.g. blood samples, liver samples. Brain samples. Muscle samples, etc.) isolated from different mice. Hence VITA could be used as a biological signature or biomarker of tissues and organs from individual organism and for their combination could be used as a universal signature/biomarker of individual organism (e.g. several cell types in the blood provide such signature of peripheral blood that could be a biomarker for particular individual).

VITA score from cross-sectional PBMC samples collected at different age points categories provides accurate prediction of the maximum lifespan for C57Bl/6 mouse. VITA score from longitudinal PBMC samples collected at different age pointed from an individual organism provides accurate prediction of the maximum lifespan for that individual providing no additional perturbations in the future. VITA score is increased following interventions that accelerate aging (e.g. chemotherapy). Therefore, VITA score could be used to monitor the adverse interventions for human health, for instance through the analysis of blood/PBMC. VITA score is decreased following interventions that slow down aging (e.g. caloric restriction). Therefore, VITA score could be used to monitor the beneficial interventions for human health, for instance through the analysis of blood/PBMC.

### EXAMPLES

### Example 1: Algorithm for Data Flow and Processing

The advent of high-content screening techniques allows for system wide application of imaging strategies to a variety of cell types and is ideally suited to probing differentiation as it permits robust identification of subpopulations of cells. The accessibility of these techniques has spurred major developments in the fields of systems biology and provides ideal tools to further understanding of the role of epigenetic modifications in the differentiation of stem and tumor initiating cells. The MIEL platform used in this study is built from several basic modules (FIG. 1 of International Publication No. WO 2022/120219A1, which is incorporated by reference herein in its entirety). The first step, segmentation, involves the identification of individual cells or in tissues of sub-domains comprised of clusters of cells.

The segments are then passed to software that makes a series of measurements of the various features. Currently morphological (roundness, area, invaginations, etc.), co-localization (overlap, correlation, etc.) and texture (bumpiness, gradients, etc.) features are measured for each cell (or segmented structure) or sub-cellular compartment (cytoplasm, nucleus, etc.). In total >500 features are measured for each cell, many of which are statistical and therefore difficult to appreciate visually (e.g., Haralick features and threshold adjacency statistics).

Once the features have been extracted, they are normalized, and subsets are identified to use for training (control) and testing (test). The training sets are used to train a classifier using machine learning algorithms that best separates the controls. To prevent over training the test set and training sets are mutually exclusive. This process is repeated until a subset of features is identified with high predictive value in many classifiers. This subset of features is used in a final round of machine learning to generate an optimal classification method.

### Example 2: MIEL Analysis of ESCs and NPCs

Classically, cell identity is defined by a combination of the cell's position within a tissue, its morphology, and a combination of molecular markers expressed specifically or selectively in some cells but not in others. While the cell morphology (e.g., using H&E staining) remains the most widely used criteria of cell identity by clinical pathologists across the globe, morphological criteria remain subjective and difficult to enumerate. On the other hand, the presence or the absence of a given marker is an easily distinguished parameter, naturally fueling the success of automated high-throughput screening. However, aberrant expression, false signal, or detection failure could easily lead to misinterpretation of cell identity, making presence/absence-based approaches error-prone. Fundamentally, markers impose identities based on what has already been defined by the existing markers. To overcome these limitations, the methods provided herein can distinguish cells based on the patterns that are always present in every cell—the epigenetic marks. In some instances, the absolute intensities (i.e. presence/absence of a given epigenetic mark) do not play a dominant role in pattern recognition algorithms.

This example provides detection of epigenetic of human induced pluripotent stem cells (iPSCs) and their progeny at single cell level. The present disclosure further provides that MIEL can enable accurate identification of each cell type in the iPSCs differentiation lineage and provide quantitative comparison of epigenetic variations between the cell types. In some instances, only certain epigenetic marks have distinguishable profiles in different cells. The present disclosure provides that epigenetic marks with the most pronounced difference can be the most informative for the classical chromatin immunoprecipitation combined with deep sequencing (ChIP-Seq) to generate genome-wide maps of histone modifications. Thus, in some instances, the immediate utility of MIEL can be validated by ChIP-Seq.

For ESCs and their derivatives, the methods provided herein can distinguish cell types with high accuracy (for example over about 99%), based on a single marker in addition to nuclear DAPI staining. For closely related cell types, the present disclosure provides that the combination of several epigenetic marks should provide the desired sensitivity and the accuracy in some instances. Since only several hundreds of cells are required for this approach, the methods provided herein can be applied to small numbers of cells, for example, rare tissue specific stem cells isolated.

It should be appreciated that the MIEL approach does not compete with, but compliments traditional approaches, which provide sequence specific epigenetic information, including ChIP, Q-PCR, complimentary DNA hybridization (microarrays) or Sequencing. Although in some instances MIEL doesn't have sufficient resolution to provide sequence-specific information, the present disclosure provides that the combinatorial use of multiple markers, MIEL and FRET techniques can provide loci-specific or even gene-specific resolution.

A robust protocol has been developed for differentiation of hESC into NPCs of dorsal identity that give rise to neural crest derivatives. The MIEL methods and systems provided herein can analyze human ESC and their immediate derivatives NPCs, first using H3K9me3 immunostaining and DAPI nuclear stain. Simultaneous co-staining for Oct4 in ESCs and for Nestin in NPCs is used to confirm the identity of each cell but is not used in the classification. The present disclosure provides that the heterochromatin in human ESCs is arranged in large clusters but become comparatively disperse in human NPCs. These findings parallel previous results obtained in mouse ESCs and NPCs. For all experiments, NPCs are obtained by hESCs differentiation for 1 week.

For this analysis MIEL is trained with 125 cells of each type, ESCs (manually verified to be Oct4+) and NPCs (manually verified to be Nestin+) and the 136 features that are most unaffected by the intensity (brightness) of fluorescent stains. Although NPCs are brighter on average, substantial overlap of H3K9me3 staining intensities in ESCs and NPCs precluded accurate assignment of cell identity based on intensity (FIG. 2A of International Publication No. WO 2022/120219A1, which is incorporated by reference herein in its entirety). Four features are automatically selected that gave the best cell separation. A feed-forward back-propagating Neural Network (10 nodes) is used for calculations to segregate the experimental mixture of ˜500 ESCs and NPCs. All cells are different from the training set and are individually verified to be either Oct4+ or Nestin+). FIG. 2A of International Publication No. WO 2022/120219A1, which is incorporated by reference herein in its entirety, shows that H3K9me3 fluorescence intensity doesn't efficiently separate ESCs and NPCs. Cells in the grey area cannot be ascribed to either population. MIEL classifies the cells with about 98% accuracy. 2B of International Publication No. WO 2022/120219A1, which is incorporated by reference herein in its entirety, shows Principal Component Analysis (PCA) representation. This algorithm successfully classified ESCs and NPCs with an accuracy of 98%±1% and is represented in Euclid coordinates using Principal Component Analysis (PCA) (FIG. 2B of International Publication No. WO 2022/120219A1, which is incorporated by reference herein in its entirety).

### Example 3: Combination of Different Epigenetic Markers

An independent set of ESCs and NPCs (total of about 350 cells) is stained for a combination of different epigenetic marks, H3K4me3 and H3K27me3. In contrast to the H3K9me3 staining the size of clusters is roughly similar in ESCs and NPCs precluding visual separation of these cell types. The individual marks had very different classification power; H3K4me3 mark contributed by 53% and H3K27me3 mark only by 1% to the overall separation and 46% contributed by the DAPI signal. (FIGS. 3A-3C of International Publication No. WO 2022/120219A1, which is incorporated by reference herein in its entirety). However, a combination of all signals successfully distinguished ESCs and NPCs with 98%±0.4% accuracy (FIG. 3D of International Publication No. WO 2022/120219A1, which is incorporated by reference herein in its entirety). This example demonstrates that different epigenetic marks have dramatically different classifying power and provides a clear-cut proof of principle for the enhanced accuracy when a combination of signals is used for MIEL-based cell identification.

### Example 4: H3K9Ac-Based or H3K4Me3-Based MIEL Analysis

A different type of chromatic modification is also used—acetylation of H3K9 on an independent set of about 800 individually verified ESCs (Oct4+) and NPCs (Nestin+). In contrast to H3Kme3 staining, the difference between human ESCs and NPCs is not distinguishable by naked eye. MIEL profiling is able to correctly call ESCs and NPCs with an accuracy of 99%±0.2% (FIG. 4 of International Publication No. WO 2022/120219A1, which is incorporated by reference herein in its entirety). Since nuclear structural proteins (e.g., Lamin A/C in nuclear membrane) are different in ESCs and NPCs, morphological features are used to separate ESC and NPCs. However, morphology even in combination with the computational “black box” may not be sufficient to distinguish ESCs and NPCs, testifying for the uniqueness of MIEL approach.

Overall chromatin structure is found to be rather similar in induced pluripotent cells (iPSCs) and ESCs. Similar results are seen when epigenetic landscapes are compared in different pluripotent cells on a single cell level. The MIEL methods and systems provided herein can compare NPCs derived from iPSCs and ESCs. Both hESCs and iPSCs are differentiated side by side to obtain NPCs. The H3K4me3 mark is used to immunolabel cells in addition to Oct4 for ESC and Nestin for NPCs (˜200 individually verified cells of each type). In a three-way comparison MIEL correctly classifies 88% of ESCs, whereas 90% of ESC-derived NPCs and 96% of iPSC-derived NPCs are classified as NPCs (FIG. 5 of International Publication No. WO 2022/120219A1, which is incorporated by reference herein in its entirety). Furthermore, when displayed in principal component space it is observed that both classes of NPCs are more similar to each other than either is to ESCs (FIG. 5 of International Publication No. WO 2022/120219A1, which is incorporated by reference herein in its entirety). Finally, the data suggests that the differences in the two cell types can be quantified from MIEL data.

### Example 5: MIEL Analysis of Neuron Astrocytes and Smooth Muscle

Since ESCs represent somewhat artificial cell type (only transiently present during development), the present disclosure provides that the MIEL methods and systems provided herein can be used to distinguish cell types closely juxtaposed in a normal human body. For this purpose, ESC-derived dorsal NPCs (which give rise to neural crest derivatives such as smooth muscles) are differentiated into young neurons (identified as TuJ1+ cells), glia (identified as GFAP+ cells) and smooth muscles (identified as SMA+ cells). Although these cells can be easily distinguished using specific markers visual examination of H3K4me3 staining of these cell types do not reveal any cell type-specific features. However, a three-way MIEL comparison using 900 cells (300 cells of each type), correctly identified 88% of astrocytes, 96% of neurons, and 99% of smooth muscle cells (FIG. 5 of International Publication No. WO 2022/120219A1, which is incorporated by reference herein in its entirety).

### Example 6: MIEL Analysis of Primary Human GBM Lines

Importantly, the present disclosure provides direct evidence that cancer cells, for example human glioblastoma multiform (GBM), could be accurately distinguished from normal cells such as human embryonic stem cells and human neural stem cells using MIEL approach. Human glioblastoma cells are a heterogeneous population of cells, which often express markers of different neural lineages including Nestin, GFAP and TuJ1 in the same cells challenging the discrimination of GBM cells from normal neural stem/precursor cells. H3K9me3 based MIEL analysis was used to compare 5 primary human GBM lines, hESC (H9), hESC-NPCs and human fetal NPCs (FIG. 6 of International Publication No. WO 2022/120219A1, which is incorporated by reference herein in its entirety). The average nuclear levels of H3K9me3 were variable between lines and even within individual GBM lines. However, the global pattern of H3K9me3 marked heterochromatin at a single cell level was similar (41-45% overlap) in all 5 GBM lines tested. In contrast, the pattern of H3K9me3 marked heterochromatin in human fetal NPCs was distinguished from several GBMs with 85%-100% accuracy. The pattern of H3K9me3 marked heterochromatin in GBMs (GBM 3 line) was also readily distinguished from hESC and hESC-NPCs with 91-99% accuracy. These data suggest that despite heterogeneity in nuclear levels of H3K9me3 in GBMs on a single cell level, based on a high degree of overlap uncovered by the MIEL analysis, the pattern of H3K9me3 marks is rather similar in all GBM lines tested. However, the H3K9me3 based MIEL analysis was able to readily distinguish GBM lines from human fetal NPCs, hESC-NPCs and hESCs at a single cell level.

### Example 7: Multiparametric Signature of Glioblastoma Differentiation Revealed by Imaging of Cellular Epigenetic Landscapes

Cell Culture: Monolayer cultures of patient-derived TPCs were propagated on Matrigel-coated plates in DMEM:F12 Neurobasal media (1:1; Gibco), 1% B27 supplement (Gibco), 10% BIT 9500 (StemCell Technologies), 1 mM glutamine, 20 ng/ml EGF (Chemicon), 20 ng/ml bFGF, 5 μg/ml insulin (Sigma), and 5 mM nicotinamide (Sigma). The medium was replaced every other day and the cells were enzymatically dissociated using Accutase prior to splitting. Fibroblasts, iPSCs, and iPSC-derived NPCs were cultured as previously described.

Differentiation treatment: For TPC differentiation treatments cells were cultured in DMEM:F12 Neurobasal media (1:1), 1% B27 supplement, 10% BIT 9500, 1 mM glutamine supplemented with either Bmp4 (100 ng/ml; R&D Systems) or FBS (10%).

Cell staining: Cells were rinsed with PBS and fixed in 4% paraformaldehyde in PBS for 10 min at room temperature. After blocking with PBSAT (2% BSA and 0.5% Triton X-100 in PBS) for 1 h at room temperature, the cells were incubated overnight at 4° C. with primary antibodies diluted in PBSAT. The primary antibodies are listed in Table 2, and the appropriate fluorochrome-conjugated secondary antibodies were used at 1:500 dilution. Nuclear co-staining was performed by incubating cells with Hoechst-33342 nuclear dye.

TABLE 1
Epigenetic drugs and concentration

| | Concentration |
|---|---|
| Compound | (μM) |
| CAY10603 | 10 |
| AT9283 | 10 |
| DZNep | 10 |
| RG2833 | 10 |
| Resminostat | 10 |
| Nexturastat-A | 30 |
| Cerdulatinib | 3 |
| OTX015 | 10 |
| OG-L002 | 30 |
| Olaparib | 10 |
| RG108 | 30 |
| A-966492 | 10 |
| I-BET-762 | 10 |
| GSK-LSD1-2HCl | 10 |
| Bromosporine | 10 |
| EPZ-6438 | 0.1 |
| Procainamide | 30 |
| OF-1 | 30 |
| Decitabine | 10 |
| LMK-235 | 10 |
| GSK1324726A | 10 |
| Selisistat | 10 |

TABLE 2
Primary antibodies

| | | | | Catalogue |
|---|---|---|---|---|
| Antigen | Host | Dilution | Brand | Number |
| H3K9me3 | rb | 1:250 | Active Motif | 39765 |
| H3K4me1 | ms | 1:250 | Active Motif | 39635 |
| H3K27me3 | ms | 1:250 | Active Motif | 61017 |
| H3K27ac | rb | 1:250 | Active Motif | 39133 |
| Sox2 | ms | 1:400 | R&D Systems | MAB2018 |
| Sox2 | gt | 1:250 | R&D Systems | AF2018 |
| Sall2 | Rb | 1:250 | Bethyl | A303-208A |
| Brn2 | gt | 1:250 | Santa Cruz | sc-6029 |
| Olig2 | rb | 1:500 | Millipore | AB9610 |
| GFAP | rb | 1:500 | Millipore | AB5804 |
| Oct4 | rb | 1:200 | Cell Signaling | 2840P |

RNAseq and transcriptomic analysis: Total RNA was isolated from GBM2 cells using the RNeasy Kit (Qiagen), 0.5 ug total RNA was used for isolation of mRNAs and library preparation. Library preparation and sequencing was conducted by the SBP genomics core (Sanford-Burnham NCI Cancer Center Support Grant P30 CA030199). PolyA RNA was isolated using the NEBNext® Poly(A) mRNA Magnetic Isolation Module and barcoded libraries were made using the NEBNext® Ultra II™ Directional RNA Library Prep Kit for Illumina® (NEB, Ipswich MA). Libraries were pooled and single end sequenced (1X75) on the Illumina NextSeq 500 using the High output V2 kit (Illumina). Read data was processed in BaseSpace. Reads were aligned to Homo sapiens genome (hg19) using STAR aligner with default settings. Differential transcript expression was determined using the Cufflinks Cuffdiff package. Go term enrichment analysis was conducted using PANTHER v11 using all genes identified as differentially expressed following either serum or Bmp4 treatment. For heat maps showing fold change in expression the FPKM values in each population were divided by the average FPKM values of untreated GBM2. To highlight differences in expression levels between serum and Bmp4 treated GBM2 cells the FPKM values in each sample were z-scored. Zscore=(FPKMObservation−FPKMAverage)/FPKMSD (FPKMObservation−FPKM value obtain through sequencing; FPKMAverage−average of all FPKM values in all samples for a certain gene; FPKMSD−standard deviation of FPKM values for a certain gene). Heat maps were generated using Microsoft Excel conditional formatting function.

Prestwick Chemical Library screen using Sox2 and GFAP: GBM2 cells were plated at 2000 cells/well and exposed to Prestwick compounds (10 μM) for 3 days in 384-well optical bottom assay plates (Greiner). Cells were then fixed and stained with goat polyclonal anti-Sox2 and rabbit polyclonal anti-GFAP (Table 2) antibodies followed by AlexaFluor-488- or AlexaFluor-555-conjugated secondary antibodies. The positive and negative control treatments were BMP4 (100 ng/ml) and DMSO (0.1%), respectively. DNA was counterstained with DAPI and the cytoplasmic region was identified with HCS CellMask Deep Red. Images were acquired using the Perkin Elmer Opera® QEHS. Image analysis protocols were developed with PerkinElmer Acapella® using standardized analysis building blocks and custom algorithm scripting. Specific antibody-based parameters, morphological and fluorometric parameters, and nuclei counts were extracted for the imaged region in each well. Nuclear mask was segmented based on DAPI stain, cytoplasm mask was segmented based on CellMask. Image analysis included quantification of cell count, the nuclear staining intensity of Sox2 and the cytoplasmic intensity of GFAP. These parameters were used to evaluate activity of compounds, which was scored as percent efficacy for decrease in Sox2 levels and increase in GFAP levels. The average robust Z′-scores (RZ′) is based on the Z′-score but uses the median and the median absolute deviation instead of the mean and the standard deviation. RZ′ were 0.31 and 0.29 for Sox2 and GFAP, respectively. Percent efficacy was calculated as: Percent efficacy=((Obs−NegCont)/(PosCont−NegCont))×100; Obs, intensity measured for compound; NegCont, average intensity of 32 DMSO-treated wells in each plate; PosCont−average intensity of 32 Bmp4-treated wells in each plate. Percent efficacy for each compound was calculated using only controls from the same plate. Hits were defined as compounds that yield percent efficacy values of either GFAP (increase) or Sox2 (decrease)>40, or only Sox2 decrease >100. To evaluate drug induced cytotoxicity robust z-score for the number of non-Pyknotic cells (Pyknotic cells were identified by decreased nuclear area and increased DAPI intensity) was calculated according to: RZscore=(CountObservation−CountMedian)/CountMAD where CountObservation denotes count of viable nuclei in the well; CountMedian denotes median cell count for all DMSO treated wells; CountMAD denotes median absolute difference of cell count for all DMSO treated wells).

Microscopy and image analysis: Unless stated otherwise, for MIEL analysis cells were imaged on an Opera QEHS high-content screening system (PerkinElmer) using ×40 water immersion objectives. Images collected on the Opera were analyzed using Acapella 2.6 (PerkinElmer). At least 40 fields per well were acquired and at least 2 wells per population were used. Features of nuclear morphology, fluorescence intensity inter-channel co-localization, and texture features (Image moments, Haralick, Threshold Adjacency Statistics) were calculated using custom algorithms (scripts available from www.andrewslab.ca). A full list of the features used is available from the authors. Values for each cell were generated and exported to MATLAB for further analysis. For Sall2, Olig2, Brn2, Sox2, Oct4 and GFAP immunostaining, images were captured on an IC200-KIC (Vala Sciences) using a ×20 objective. Between 3 and 8 fields per well were acquired and analyzed using Acapella 2.6 (PerkinElmer). For all nuclear markers, average intensities in nucleus or fold change in average intensity compared to untreated cells are shown. Unless stated otherwise, at least 3 wells and a minimum of 300 cells for each condition were compared using unpaired two-tailed t-test was.

MDS: The image features based profile for each cell population (e.g., cell types, treatments) was represented using a vector (center of distribution vectors) in which every element is the average value of all cells in that population for a particular feature. The vector's length is given by the number of features chosen. All vectors used to composite the MDS maps (distance maps) consisted of 524 texture features (262 per channel, 2 channels). Cell-level data in all populations together were normalized to z-scores prior to calculation of center of distribution vectors. All cells in each population were used to calculate center vectors and each population contained at least 400 cells. Transcriptomic based profile for each cell population was represented using a vector in which every element is the z-scored FPKM value for a single gene in that population. The length of the vector is given by the number of genes used to construct the profile. The Euclidean distance between all vectors (either image features or transcriptomic based) was then calculated to assemble a dissimilarity matrix (size N×N, where N is the number of populations being compared). For representation, the N×N matrix was reduced to a N×2 matrix with MDS using the MATLAB (2016a) function ‘cmdscale’ or an Excel add-on program Xlstat (Base, v19.06), and displayed as a 2D scatter plot.

Polar plots: Due to the inherent heterogeneity of TPC lines, data normalization was performed when comparing multiple treatments on several TPC lines. For this, the value of each feature for all individual cells in each line was divided by the average value obtained for that feature in the untreated population from the same cell line. Therefore, following normalization, untreated cells from all lines had the same center of distribution vector (in which all elements are equal to 1), while each treatment retained its relative distance from untreated as well as from all other treatments of the same cell line. However, as each cell line is divided by a different value, the distance vectors originating from two different lines represent the change in feature values induced by treatment, rather than the absolute feature values. Therefore, following MDS, the results are shown on a polar plot to indicate that the various treatments induce similar feature value changes in multiple lines rather than similar absolute values. As a result, direction and distance to the origin are comparable between lines while distances directly between points are not.

SVM classification: SVM classification was conducted as previously described (26). Cell-level data in all populations (minimum 400 cells per population) together were normalized to z-scores and a subset of cells from each of the populations being classified was randomly chosen as the training set (subset size is at least 100×the number of populations being classified). The training set was used to train a SVM classifier (MATLAB function ‘svmtrain’). The remaining cells (test set) were then classified using the SVM-derived classifier to assess the accuracy of classification (MATLAB function ‘svmclassify’). Here, the accuracy of all pairwise classifications is given as the average accuracy calculated for each of the populations. To utilize classification to determine the similarity of multiple cell populations, classified known populations (such as different treatments or cell fates) to generate known ‘bins’ and then used the same classifiers on the unknown population to categorize each cell.

Prestwick Chemical Library screen using H3K27me3 and H3K27ac: GBM2 cells were plated at 2000 cells/well and exposed to Prestwick compounds (3 μM) for 3 days in 384-well optical bottom assay plates (PerkinElmer). Cells were then fixed and stained with rabbit polyclonal anti-H3K27ac and mouse monoclonal anti-H3K27me3 (Table 2) antibodies followed by AlexaFluor-488- or AlexaFluor-555-conjugated secondary antibodies. The positive control treatments were BMP4 (100 ng/ml) and serum (10%), negative controls were DMSO (0.1%). DNA was counterstained with Hoechst. Images were acquired using the Perkin Elmer Opera® QEHS. MIEL analysis was conducted as described above. The robust Z′-score (RZ′) is based on the Z′-score described in (32), but uses the median (<x>) and the robust standard deviation (rSD) based on the median absolute deviation (MAD) instead of the mean and the standard deviation. Briefly, the DMSO-(negative) and BMP4- or serum-treated (positive) control wells was used to establish the signatures corresponding to undifferentiated (DMSO) and differentiated (BMP4 and/or serum) GBM cells. Using these signatures, the cells in each well were classified to obtain the population fraction of differentiated GBM cells per well. These values are used to calculate the medians and rSDs for all DMSO (<x>neg and rSDneg) and all BMP- or serum-treated (<x>pos and rSDpos) wells. The RZ′ value is calculated as follows: RZ′=1−(3*rSDpos+3*rSDneg)/|<x>pos−<x>neg| with rSD=MAD*1.4826 and MAD=<|x−<x>|>. RZ′ values are calculated for DMSO vs. Bmp4 treated, DMSO vs. serum treated, and DMSO vs. pooled Bmp4 and Serum treated wells. The Signal-to-Background (S/B) uses the formula: μpos/μneg where μ is the average of the differentiated population fractions for all treated (Bmp4, Serum) or control (DMSO) wells.

Correlation of transcriptomic and image-based profiles: Euclidean distance between untreated, serum or Bmp4 treated GBM2 cells (triplicates for each) was calculated using either transcriptomic data (FPKM) or texture features. Pearson's correlation coefficient (R) was transformed to a t-value using the formula (t=R×SQRT(N−2)/SQRT(1−R2) where N is the number of samples, and R is Pearson correlation coefficient, and the p-value was calculated using Excel tdist(t) function. For compound prioritization, the Euclidean distance between compound treated and serum or Bmp4 treated GBM2 cells was calculated based on either transcriptomic data (FPKM) or image features. The average distance to both serum and Bmp4 treatments was normalized to the average distance of untreated cells to serum and Bmp4.

Brief Treatment with Serum or Bmp4 Initiates TPC Differentiation

A comparative analysis of gene expression changes in TPCs following short serum or Bmp4 treatment, which is relevant to the high-throughput screening objective, has not been conducted. Several GBM cell lines were treated for 3 days with serum or Bmp4 and then quantified expression of core transcription factors previously shown to determine the transcriptomic program of TPCs. Immunostaining revealed that the 4 transcription factors Sox2, Sall2, Brn2 and Olig2 were down regulated by both serum and Bmp4 in a cell line dependent manner (FIG. 11A of International Publication No. WO 2022/120219A1, which is incorporated by reference herein in its entirety). After 3 days of treatment, the growth rate of TPCs was reduced by both serum and Bmp4 (FIG. 111B of International Publication No. WO 2022/120219A1, which is incorporated by reference herein in its entirety). RNAseq analysis of serum and Bmp4 treated GBM2 cells revealed that 3 days treatment reduced (vs untreated cells) the expression of most genes previously found to constitute the transcriptomic stemness signature (FIG. 11C of International Publication No. WO 2022/120219A1, which is incorporated by reference herein in its entirety). To identify the cellular processes altered by these treatments, differential expression analysis was conducted. It was found that expression of 4852 genes was significantly altered (p<0.01 and −1.5<Fold Change>1.5) by either serum or Bmp4 treatment. Gene Ontology (GO) analysis of these altered genes indicated enrichment in multiple GO categories consistent with initiation of TPC differentiation—including cell cycle, cellular morphogenesis associated with differentiation, differentiation in neuronal lineages, histone modification, and chromatin organization (FIG. 12 of International Publication No. WO 2022/120219A1, which is incorporated by reference herein in its entirety). Taken together, these results demonstrate that a 3 day treatment with serum or Bmp4 is sufficient to result in transcriptomic changes characteristic of TPC differentiation.

Sox2- and GFAP-Based Screening Doesn't Prioritize Inducers of TPC Differentiation

It is contemplated that Sox2 function is required for maintenance of TPCs and that its knockdown induces TPC differentiation. Thus, Sox2 was selected as a marker of the TPC state. For the differentiated state, GFAP was selected an astrocytic marker previously shown to be upregulated following differentiation of TPCs. It was confirmed that Bmp4, but not serum, treatment also increased GFAP expression (FIG. 11A of International Publication No. WO 2022/120219A1, which is incorporated by reference herein in its entirety).

Among several GBMs tested, the GBM2 line exhibited the largest reduction in Sox2 and increase in GFAP and was selected for screening. GBM2 TPCs were plated in 384-well plates, treated with the Prestwick library compounds (10 μM, 1200 molecules) for 3 days, fixed, and then immunostained for Sox2 and GFAP. Hits were defined as compounds that increased GFAP and decrease Sox2 by more than 40% or any compounds that decrease Sox2 alone by more than 100%; Bmp4 was used as a positive control (FIG. 7A of International Publication No. WO 2022/120219A1, which is incorporated by reference herein in its entirety). 19 hits were detected (FIG. 13 of International Publication No. WO 2022/120219A1, which is incorporated by reference herein in its entirety), however, reduced cell viability and the presence of pyknotic nuclei indicated apparent cytotoxicity of the most hits (z-score for viable cell count less than −4 compared with −2.33 for Bmp4; FIG. 13 of International Publication No. WO 2022/120219A1, which is incorporated by reference herein in its entirety). Thus, the hit compounds were retested at lower concentrations (3, 1 and 0.3 μM) and observed that a 3 days treatment with 0.3 μM Digitoxigenin, a Na⁺/K⁺ ATPase inhibitor, was able reduce Sox2 expression while maintaining growth rate and Ki67 expression levels similar to Bmp4 (FIG. 7B-C and supplementary FIG. 20A of International Publication No. WO 2022/120219A1, which is incorporated by reference herein in its entirety). A related compound Digoxin also reduced Sox2 expression but induced a stronger reduction in growth rate (FIG. 7B-C and supplementary FIG. 20A of International Publication No. WO 2022/120219A1, which is incorporated by reference herein in its entirety); both Digitoxigenin and Digoxin were able to downregulate expression of the core transcription factors similar to serum and Bmp4 (FIG. 7C of International Publication No. WO 2022/120219A1, which is incorporated by reference herein in its entirety). To validate these results using a “gold standard” of cell fate analysis, a whole genome expression analysis of treated GBM2 TPCs was conducted. To test whether Digitoxigenin and Digoxin increased transcriptomic similarity of treated cells to serum or Bmp4 treated cells, FPKM values of all expressed genes (FPKM>1) were used to calculate the Euclidean distance between drug and serum or Bmp4 treated cells. Neither Digitoxigenin nor Digoxin reduced the distance of treated cells to the desired state (FIG. 7D of International Publication No. WO 2022/120219A1, which is incorporated by reference herein in its entirety). Several representative GO terms illustrate the gene expression changes induced by digoxin and digitoxigenin which were markedly different from those induced by serum or BMP4 (FIG. 7E of International Publication No. WO 2022/120219A1, which is incorporated by reference herein in its entirety). It implicates that the 2 top hits didn't induce desirable TPC fate change, despite downregulation of some core transcription factors essential for TPC propagation. Further such results emphasize the need of developing novel approaches to interrogate TPC differentiation, which are compatible with the high-throughput screening and align well with the entire transcriptome analysis.

Development of MIEL Platform

A novel phenotypic screening platform, which interrogates the epigenetic landscape at single cell level using imaged-based machine learning, was developed. MIEL takes advantage of epigenetic marks such as histone methylation and acetylation, which are always present in eukaryotic nuclei and can be revealed by immunostaining. MIEL analyzes the immunolabeling patterns of epigenetic marks at the single-cell level—using conventional image analysis methods for segmentation of nuclei, feature extraction and previously described machine learning algorithms (FIG. 8A and FIGS. 14A-14B of International Publication No. WO 2022/120219A1, which is incorporated by reference herein in its entirety).

Primarily, 4 histone modifications were used: H3K27me3 and H3K9me3, which are associated with condensed (closed) facultative and constitutive heterochromatin, respectively; H3K27ac, associated with transcriptionally active (open) areas of chromatin, especially at promoter and enhancer regions; and H3K4me1, associated with enhancers and other chromatin regions. To focus the learning algorithm on the intrinsic pattern of epigenetic marks, the intensity and nuclear morphology features were discarded and only texture-associated features (e.g., Haralick's texture features, threshold adjacency statistics, and radial features) were used for multivariate analysis. Further observed patterns were interpreted as a 2D projection of the 3D topological distribution of a given epigenetic mark in the nucleus. Although this representation degrades the spatial information, the resulting 2D textures, such as foci of high and low intensity, are visually apparent in the computer-enhanced images (FIG. 14A of International Publication No. WO 2022/120219A1, which is incorporated by reference herein in its entirety).

MIEL Analysis Provides Signatures of Cell Fates

MIEL was further developed to distinguish between differentiated and undifferentiated TPCs, and to obtain a multiparametric signature of differentiated TPCs. To validate MIEL's ability to discriminate between different cellular states/fates involving major changes in chromatin organization (e.g., reprogramming and differentiation), 3 cell types were analyzed: primary human fibroblasts isolated from 3 donors (WT-61, WT-101, WT-126), induced pluripotent stem cell (iPSC) lines derived from the fibroblasts, and neural progenitor cell (NPC) lines differentiated from the iPSCs, therefore providing genetically matching fibroblasts, iPSC and NPC cells. Cellular identities of the 3 cell types were verified by immunofluorescence (FIG. 8B of International Publication No. WO 2022/120219A1, which is incorporated by reference herein in its entirety).

The 9 cell lines were immunostained for H3K4me1 and H3K9me3 marks, chosen based on major pattern alteration of these marks during differentiation. Note that immunostaining for H3K27ac and H3K27me3 marks produced a similar distance map (FIG. 15A of International Publication No. WO 2022/120219A1, which is incorporated by reference herein in its entirety). Both pairs of epigenetic marks were used interchangeably for further analysis. Images and extracted image features were segmented, and multivariate centroids were calculated for each cell population. Multi-dimension scaling (MDS) was employed to reduce 524 texture features into 2D and plotted to visualize the relative Euclidean distance between various cell populations (referred to as the “distance map”). Fibroblasts, iPSCs and NPCs each segregate to form 3 visually distinct territories (FIG. 8C of International Publication No. WO 2022/120219A1, which is incorporated by reference herein in its entirety).

To determine whether it was possible to discriminate between individual cells with different fates, a Support Vector Machine (SVM) classifier was trained using fibroblasts, iPSCs, and NPCs derived from donor WT-61. This classifier accurately identified 79% of fibroblasts, 79% of iPSCs and 97% of NPCs (overall accuracy 85% FIG. 8D of International Publication No. WO 2022/120219A1, which is incorporated by reference herein in its entirety; overall accuracy for H3K27ac and H3K27me3 based classification was 82%, FIG. 15B of International Publication No. WO 2022/120219A1, which is incorporated by reference herein in its entirety). Similar results were obtained when the classifier was trained using cell lines from the other 2 donors. A classifier derived by pooling WT-61, WT-101, and WT-126 cells correctly identified 89% of fibroblasts, 90% of iPSCs and 94% of NPCs (overall accuracy 91% FIG. 8E of International Publication No. WO 2022/120219A1, which is incorporated by reference herein in its entirety; overall accuracy for H3K27ac and H3K27me3 based classification was 90% FIG. 15C of International Publication No. WO 2022/120219A1, which is incorporated by reference herein in its entirety). Furthermore, a direct pairwise classification distinguished different genetic backgrounds with 74% (FIG. 15D of International Publication No. WO 2022/120219A1, which is incorporated by reference herein in its entirety). Additionally, MIEL analysis was able to discriminate between various primary hematopoietic cell types freshly isolated from mouse bone marrow suggesting that such analysis is not a cell culture artifact (FIG. 16 of International Publication No. WO 2022/120219A1, which is incorporated by reference herein in its entirety).

These results suggest that MIEL can be used to distinguish between different differentiation states based on their single-cell epigenetic landscapes. Furthermore, multiparametric signatures was derived for several cell types (e.g., fibroblasts, iPSCs, NPCs) that discriminate each cell type from the others.

MIEL Determines Signature of TPC Differentiation.

To begin deriving the signature of GBM differentiation, MIEL's ability to distinguish TPCs and differentiated glioma cells (DGCs), derived from the same primary human GBMs was tested. Three TPC/DGC pairs were derived in parallel from 3 genetically distinct GBM tumor samples (MGG4, MGG6, and MGG8) over a 3-month period using either serum-free FGF/EGF conditions for TPCs or 10% serum for DGCs. MIEL analysis distinguished TPCs from their corresponding DGC lines with an average accuracy of 83%, using any of the 4 epigenetic marks tested (H3K27me3, H3K9me3, H3K27ac, and H3K4me1; FIGS. 9A-9B of International Publication No. WO 2022/120219A1, which is incorporated by reference herein in its entirety). An SVM classifier derived from images of the MGG4 TPC/DGC pair separated all 3 TPC/DGC pairs with 88% average accuracy, providing proof of principle for the derivation of a signature for non-tumorigenic cells obtained following serum differentiation of primary GBM cells (FIG. 9C of International Publication No. WO 2022/120219A1, which is incorporated by reference herein in its entirety).

Next, it was tested whether shorter serum treatment (compatible with the screening protocols) would induce detectable epigenetic alterations. 4 low-passage primary TPCs were treated for 9 days with 10% serum and compared their epigenetic landscape to that of untreated cells and “terminally” differentiated DGCs. MDS was used to visualize the relative Euclidean distance between populations. While untreated cells were quite heterogeneous, serum treatment reduced the distance from all TPC centroids to DGC centroids (n=4 cell lines, p<0.05; unpaired two-tailed t-test; FIGS. 17A-17B of International Publication No. WO 2022/120219A1, which is incorporated by reference herein in its entirety). These concordant results obtained with 7 independent human GBM lines attest to the robustness of the serum-induced epigenetic changes detected by MIEL and suggesting that similar epigenetic patterns may exist in other differentiated GBM lines.

To compare the outcomes of several experiments using multiple GBM lines and treatments, a normalization procedure was developed to compare the changes in feature space induced by treatments by bringing together the centroids of all TPCs (including MGG-TPCs). The results are then displayed using a polar plot in which treatments for each cell line are represented as vectors with a magnitude—rho (the distance from the center) and directionality given by the angular coordinate theta. For all GBM lines, the magnitude and direction of changes induced by 9-day serum treatment were comparable and similar to that seen in the MGG TPC/DGC pairs (rho: FBS-9d=10.1±1.0, DGCs=10.0±1.64; theta: FBS-9d=−2.4±0.2, DGCs=−2.5±0.4); three-day serum treatment induced feature changes comparable in direction, but not magnitude (rho: FBS-3d=4.1±1.0; theta: FBS-3d=−2.2±0.5; FIG. 9D and FIG. 17C of International Publication No. WO 2022/120219A1, which is incorporated by reference herein in its entirety).

To test the accuracy of separating TPCs and DGCs at the single-cell level, an SVM classifier was generated and trained on texture features derived from a random subset of H3K27ac and H3K27me3 images of TPCs and DGCs (MGG4, 6, 8 pooled for both). The classifier separated pooled TPCs from pooled DGCs with 92.8% accuracy (FIG. 9E of International Publication No. WO 2022/120219A1, which is incorporated by reference herein in its entirety) and categorized 76% of untreated cells as TPCs and 69% of serum-treated cells as DGCs (FIG. 9E of International Publication No. WO 2022/120219A1, which is incorporated by reference herein in its entirety).

These experiments demonstrate that MIEL is suitable to determine a signature of differentiated GBM cells across multiple genetic backgrounds. Furthermore, MIEL can detect serum-induced changes in GBM epigenetic pattern within several days to monitor the progress of TPC differentiation in a timeframe suitable for high content screening.

Validation of MIEL Signature Using Global Transcriptomic Analysis

It is contemplated that distinct features of GBM differentiation induced with BMP compared to serum. Distinct expression changes were observed, including differences in expression of genes regulating chromatin organization and histone modifications (FIG. 18 of International Publication No. WO 2022/120219A1, which is incorporated by reference herein in its entirety), between serum- and Bmp4-induced GBM differentiation. Therefore, whether MIEL approach could distinguish these differentiation modalities, in particular at the early time points, was investigated.

Four genetically distinct GBM lines were treated for 2 days with serum or BMP4 and conducted MIEL analysis using H3K9me3 and H3K4me1 marks. To visualize the changes induced by each treatment, used polar plot normalization was used, as described above. Indeed, it was observed that serum and BMP4 induce distinct epigenetic changes as detected by MIEL for each GBM line tested (FIG. 9F of International Publication No. WO 2022/120219A1, which is incorporated by reference herein in its entirety).

Global gene expression profile represents a gold standard to define the cellular state. Therefore, it was tested whether the relative distances between distinct cellular states, for instance, untreated GBM cells, serum treated, and BMP treated GBM cells correlate using MIEL-based metrics and global gene expression-based metrics. Untreated and 3 days serum or Bmp4 treated GBM2 TPCs were sequenced, and all genes with FPKM>1 in at least one cell population were used to calculate the Euclidean distance matrix between all cell populations. FPKM-based distances were then correlated to image texture feature-based distances. The resulting Pearson correlation coefficient of R=0.93 suggests a high correlation between these 2 metrics (FIGS. 9G-9H of International Publication No. WO 2022/120219A1, which is incorporated by reference herein in its entirety) and validates the robustness of MIEL approach for the analysis of GBM differentiation.

These experiments demonstrate that MILE is capable of distinguishing closely related GBM differentiation routes induced by serum or BMP. These results validate the robustness and accuracy of MIEL-based analysis of epigenetic patterns using conventional global gene expression approach.

MIEL Prioritizes Compounds Based on Serum/Bmp4 Signature of GBM Differentiation

To test whether MIEL can prioritize compounds based on serum/Bmp4 signature of GBM differentiation, the Prestwick compound library (at lower concentration, 3 μM to minimize toxicity) was re-screened. GBM2 TPCs were plated on 384-well plates, treated for 3 days with Prestwick compounds fixed, and then immunostained for H3K27ac and H3K27me3. GBM2 cells treated with DMSO, serum, BMP4, or compound were compared within the same plate (to avoid imaging artifacts and normalization issues). To identify compounds inducing epigenetic changes reminiscent of serum/BMP4-induced differentiation, pairwise classification of DMSO- and either serum- or BMP4-treated cells was conducted. Because both serum and BMP4 induce TPC differentiation and reduce tumorigenicity, compounds were selected to induce at least 50% of the cells to be classified as either serum- or BMP4-treated. Euclidean distance was calculated between these candidate compounds and serum/BMP4 treated cells—selecting compounds for which the distance to one or both treatments was less than the distance between DMSO and that treatment. This screen yielded 20 candidate compounds (FIG. 19A of International Publication No. WO 2022/120219A1, which is incorporated by reference herein in its entirety), of which 15 belonged to 1 of the following 4 categories: Na/K-ATPase inhibitors of the digoxin family, molecules that disrupt microtubule formation or stability, topoisomerase inhibitors, and nucleotide analogues that disrupt DNA synthesis.

Of these 15 candidate compounds, the 2 top compounds from each of the 4 categories (8 total) were chosen for further analysis. For each of the 8 compounds, pairwise classification of untreated cells and either serum- or Bmp4-treated cells was used to identify the lowest concentration where at least 50% of cells are categorized as treated (FIG. 19B of International Publication No. WO 2022/120219A1, which is incorporated by reference herein in its entirety). These concentrations were used for all subsequent experiments. Because most of these compounds are known for their cytotoxic effects, the growth rates of drug-treated GBM cells were verified. With the exception of Digoxin, which was cytostatic, treatment with drugs resulted in the growth rates comparable with that induced by serum/BMP4 treatment (FIG. 20A of International Publication No. WO 2022/120219A1, which is incorporated by reference herein in its entirety). Immunofluorescence was used to test for the expression of the core TPC transcription factors (Sox2, Sall2, Brn2 and Olig2). With the exception of Trifluridine all compounds induced statistically significant reductions in Sox2, but no reduction in the other core factors (FIG. 20B of International Publication No. WO 2022/120219A1, which is incorporated by reference herein in its entirety; see FIG. 7C of International Publication No. WO 2022/120219A1, which is incorporated by reference herein in its entirety, for Digoxin and Digitoxigenin).

The next question is whether MIEL can prioritize compounds according to their effect on TPC as judged by the transcriptomic changes induced by these compounds. GBM2 cells were treated with DMSO (negative control), serum or Bmp4 (positive controls), or 1 of the 8 candidate compounds; after 3 days, RNA was extracted and sequenced. Transcriptomic profiles of the 8 compounds were ranked according to average Euclidean distance (based on FPKM values for all expressed genes) from serum/BMP4-treated cells. To safeguard against potential artefacts of cytotoxicity, gene expression-based ranking was compared with the measured cellular growth rates for all drug treatments. Indeed, no positive correlation was revealed (FIG. 20C of International Publication No. WO 2022/120219A1, which is incorporated by reference herein in its entirety).

Next, the levels of Sox2 expression under all treatment conditions were compared to determine whether this metric is informative for identifying the drugs that best mimic serum/BMP4 treatment. No positive correlation was observed between Sox2 expression levels and the transcriptomic-based rankings (FIG. 10A of International Publication No. WO 2022/120219A1, which is incorporated by reference herein in its entirety), suggesting that SOX2 level alone is insufficient to stratify the compounds.

To compare MIEL based signatures to the transcriptomic profile, a comprehensive readout of the epigenetic landscape of treated cells was obtained. MIEL analysis was conducted using an additional set of histone modifications including H3K9me3 and H3K4me1 marks. MIEL readouts of cells treated with the 8 drugs were ranked according to average Euclidean distance from serum- or Bmp4-treated cells (calculated using texture features derived from images of 4 histone modifications). Comparison of the MIEL-based metric with the gene expression-based metric revealed a high degree of positive correlation between MIEL- and gene expression-based rankings (Pearson correlation coefficient R=0.92, p<0.001, one side t-test, n=6, FIG. 10B of International Publication No. WO 2022/120219A1, which is incorporated by reference herein in its entirety). To further visualize these results, heat-maps were constructed to depict fold change in expression of genes associated with several GO terms enriched by serum and Bmp4 treatments. The top candidate, etoposide, altered expression of a large portion of genes in a similar fashion to that of serum and BMP4; in contrast, the lowest-ranking candidate, digoxin, induced gene expression changes that were rather different from serum and BMP4 (FIG. 10C of International Publication No. WO 2022/120219A1, which is incorporated by reference herein in its entirety). These results suggest a robust correlation between MIEL- and global expression-based readouts of GBM differentiation, therefore validating MIEL approach for prioritizing hits in high-content screening aimed at identifying small molecules that mimic the effect of serum/BMP4 on GBM differentiation.

Cytotoxic drugs have had limited success treating GBM; therefore, in this example, alternative approach—inducing GBM differentiation—was used. Previously established biologicals such as serum and BMP4 known to induce GBM differentiation in culture were analyzed to establish signatures of such differentiated GBM cells based on the pattern of epigenetic marks that could be applied across several genetic backgrounds. This is the first time that GBM differentiation signature suitable for high-throughput drug screening could be obtained. Indeed, the results of previous studies using bulk analysis of GBM or single-cell sequencing could not be readily applied for high-throughput screening. Prestwick chemical library of 1200 approved drugs was analyzed to validate MIEL's ability to select and prioritize small molecules, which mimic the effect of serum and BMP4 using global gene expression profiling. Surprisingly, it was observed that the degree of reduction in endogenous SOX2 protein levels following drug treatment did not correlate with the degree of differentiation assessed by global gene expression. In contrast, the MIEL-based metrics did correlate with the degree of differentiation assessed by global gene expression. Therefore, MIEL can be readily applied to screen large compound libraries using a reference signatures of GBM differentiation (e.g., serum or BMP4) to identify novel small molecules that mimic the effect of serum or BMP4 on GBM.

Accurately defining the identity of a cell is of fundamental importance to cell biology. Currently, this is done by assessing the presence or absence of a panel of experimentally verified lineage-specific markers. However, these markers require manual and arbitrary thresholding, which could be confusing and potentially contributes to multiple challenges of reproducibility in biomedical science. These concerns are alleviated by expression profiles that use hundreds of genes to assign a specific gene signature to a given cell type. However, at a single-cell level, expression profiling becomes stochastic, and is difficult to apply to high-throughput analysis in a cost- and time-effective manner. Phenotypic drug screening is an emerging technology that is revolutionizing drug discovery. This example discloses a new method for phenotypic identification of a cell state that offers reproducibility, single-cell resolution and scalability for high-content screening. MIEL takes advantage of robust and reproducible patterns of epigenetic marks that are always present in every eukaryotic cell. Using MIEL, the unique signatures were defined of various cell types in culture such as fibroblasts, iPSCs, NPCs as well as primary cells isolated from mouse bone marrow (T cells, B cells, monocytes, and hematopoietic stem/progenitor cells) enabling their identification with over 80% accuracy.

It is becoming increasingly apparent that nuclear chromatin is spatially organized relative to the gene expression pattern; for example, CTCF proteins play a role in dictating boundaries of topologically associated domains (TADs). TADs are thought to parse chromatin into loosely defined active (euchromatin) and inactive (heterochromatin) domains, reciprocating particular patterns of gene activity. It is tempting to conjecture that the 2D epigenetic landscapes, which can be imaged at the single-cell level by MIEL, define the state of chromatin and the gene expression pattern (i.e., a cell's molecular identity). While other phenotypic screens are based on diverse strategies for labeling cellular compartments (e.g., nucleus, membranes or mitochondria), MIEL is rooted in the spatial organization of epigenetic marks. The ability of MIEL to distinguish between multiple cell fates with high accuracy indicates that the topology of epigenetic marks might be used as a proxy for single cell state and function. Providing MIEL can be adapted to analyze epigenetic landscapes in 3D, it might offer unique insights into cellular heterogeneity during development and aging and enable in situ analysis of epigenetic variations in normal human tissues and various pathologies including cancer.

### Example 8: Improving Drug Discovery Using Image-Based Multiparametric Analysis of the Epigenetic Landscape

Cell Culture: Monolayer cultures of patient-derived GMB TPCs were propagated on Matrigel-coated plates in DMEM:F12 Neurobasal Medium (1:1; Gibco), 1% B27 supplement (Gibco), 10% BIT 9500 (StemCell Technologies), 1 mM glutamine, 20 ng/ml EGF (Chemicon), 20 ng/ml bFGF, 5 μg/ml insulin (Sigma), and 5 mM nicotinamide (Sigma). The medium was replaced every other day and the cells were enzymatically dissociated using Accutase prior to splitting. Fibroblasts, iPSCs, and iPSC-derived NPCs were cultured.

Differentiation treatment: For TPC differentiation treatments cells were cultured in DMEM:F12 Neurobasal Medium (1:1), 1% B27 supplement, 10% BIT 9500, 1 mM glutamine supplemented with either Bmp4 (100 ng/ml; R&D Systems) or FBS (10%).

Immunofluorescence: Cells were rinsed with PBS and fixed in 4% paraformaldehyde in PBS for 10 min at room temperature. After blocking with PBSAT (2% BSA and 0.5% Triton X-100 in PBS) for 1 hour at room temperature, the cells were incubated overnight at 4° C. with primary antibodies diluted in PBSAT. The appropriate fluorochrome-conjugated secondary antibodies were used at 1:500 dilution. Nuclear co-staining was performed by incubating cells with either Hoechst-33342 or DAPI nuclear dyes.

Microscopy and image analysis: For MIEL analysis, cells were imaged on either an Opera QEHS high-content screening system (PerkinElmer) using ×40 water immersion objectives or an IC200-KIC (Vala Sciences) using a ×20 objective. Images collected were analyzed using Acapella 2.6 (PerkinElmer). At least 40 fields/well for Opera and 5 fields/well for IC200 were acquired and at least 2 wells per population were used. Features of nuclear morphology, fluorescence intensity inter-channel co-localization, and texture features (Image moments, Haralick, Threshold Adjacency Statistics) were calculated using custom algorithms. A full list of the features used is available from the authors. Values for each cell were generated and exported to Microsoft Excel or MATLAB for further analysis. For Sall2, Olig2, Brn2, Sox2, Oct4, and GFAP immunostaining, images were captured on an IC200-KIC (Vala Sciences) using a ×20 objective. Between 3 and 8 fields per well were acquired and analyzed using Acapella 2.6 (PerkinElmer). For all nuclear markers, average intensities in nucleus or fold change compared to untreated cells are shown. Unless stated otherwise, at least 3 wells and a minimum of 300 cells for each condition were compared using the unpaired two-tailed t-test.

Data processing: The image features-based profile for each cell population (e.g., cell types, treatments, technical repetition) was represented using a vector (center of distribution vectors) in which every element is the average value of all cells in that population for a particular feature. The vector's length is given by the number of features chosen (262 per histone modification). Raw feature values were normalized by z-scoring to the average and standard deviation of all populations being compared. All cells in each population were used to calculate center vectors, and each population contained at least 50 cells. Activity level for each drug was determined by calculating the distance from DMSO. For this, feature values of all DMSO replicates center vectors were used to calculate the DMSO center vector. Euclidean distance of each compound and each DMSO replicate to the DMSO center vector was calculated. Distances were z-scored to the average distance and standard deviation of DMSO replicates from the DMSO center vector. Transcriptomic-based profile for each cell population was represented using a vector in which every element is the z-scored FPKM value for a single gene in that population. The length of the vector is given by the number of genes used to construct the profile.

Multidimensional scaling—MDS: The Euclidean distance between all vectors (either image features or transcriptomic based) was calculated to assemble a dissimilarity matrix (size N×N, where N is the number of populations being compared). For representation, the N×N matrix was reduced to a N×2 matrix with MDS using the Excel add-on program Xlstat (Base, v19.06), and displayed as a 2D scatter plot.

Discriminant Analysis: Quadratic discriminant analysis was conducted using the Excel add-on program xlstat (Base, v19.06). The model was generated in a stepwise (forward) approach using default parameters. All features derived from images of tested histone modification were used for analysis following normalization by z-score. Features displaying multicollinearity were reduced. Model training was done using multiple DMSO replicates and at least 2 replicates from each cell-line or drug treatment. The model was tested on at least 8 DMSO replicates and at least 1 replicate from each cell line or treatment.

SVM classification: SVM classification was conducted as previously described (30). Cell-level data in total populations (minimum 400 cells per population) were normalized to z-scores, and a subset of cells from each population being classified was randomly chosen as the training set (subset size at least 100× the population number being classified). The training set was used for a SVM classifier (MATLAB svmtrain function). The remaining cells (test set) were then classified using the SVM-derived classifier to assess the accuracy of classification (MATLAB svmclassify function). Here, the accuracy of all pairwise classifications was given as the average accuracy calculated for each population. To classify the similarity of multiple cell populations, known populations (e.g., different treatments or cell fates) were classified to generate known bins and then used the same classifiers on the unknown population to categorize each cell.

Epigenetic Drug Screening: GBM2 cells were plated at 4000 cells/well and exposed to epigenetic compounds at 10 μM for 1 day in 384-well optical bottom assay plates (PerkinElmer). Negative control was DMSO (0.1%), 48 DMSO replicates per plate, 3 technical replicates (wells) were treated per compound. Cells were fixed and stained with histone modification-specific antibodies (H3K27ac & H3K27me3, H3K9me3, H3K4me1) and AlexaFluor-488- or AlexaFluor-555-conjugated secondary antibodies. DNA was stained with DAPI followed by imaging and feature extraction. To compare data from multiple plates, average feature values in each plate were normalized to DMSO. Here, feature values of all DMSO replicates center vectors in each plate, then were used to calculate the plate-wise DMSO vector. Raw feature values for all center vectors of all populations in each plate were normalized to the plate-wise DMSO vector; normalized feature values were z-scored as above. To identify active compounds, activity level for each compound was calculated as above, and active compounds were defined as compounds for which activity z-score was >3. Compounds reducing the number of imaged cells per well below 50 were considered toxic and excluded from analysis.

Concentration Curves: GBM2 cells were plated and stained as above. For each compound, cells were treated at 0.1, 0.3, 1.0, 3.0, 10.0 μM. Activity levels were calculated as above. Average cell count was calculated across the replicates for each compound to compare epigenetic changes and toxicity. Cell counts were z-scored against the average and standard deviation of all DMSO replicates. Distances (z-scored) and cell counts (z-scored) were averaged for each functional class at each concentration.

RNAseq and transcriptomic analysis: Total RNA was isolated from GBM2 cells using the RNeasy Kit (Qiagen), 0.25 μg total RNA was used to isolate mRNAs and for library preparation. Library preparation and sequencing were conducted by the SBP genomics core (Sanford-Burnham NCI Cancer Center Support Grant P30 CA030199). PolyA RNA was isolated using the NEBNext® Poly(A) mRNA Magnetic Isolation Module, and barcoded libraries were made using the NEBNext® Ultra II™ Directional RNA Library Prep Kit for Illumina® (NEB, Ipswich MA). Libraries were pooled and single-end sequenced (1×75) on the Illumina NextSeq 500 using the High-Output V2 kit (Illumina). Read data, processed in BaseSpace, were aligned to Homo sapiens genome (hg19) using STAR aligner with default settings. Differential transcript expression was determined using the Cufflinks Cuffdiff package. For heat maps showing fold change in expression, FPKM values in each HDACi-treated population were divided by the average FPKM values of DMSO-treated GBM2 and values shown as log 2 of the ratio. Go enrichment analysis was conducted using PANTHER v11 using all genes identified as differentially expressed following either serum or Bmp4 treatment. To highlight differences in expression levels between serum- and Bmp4-treated GBM2 cells, FPKM values in each sample were z-scored. Zscore=(FPKMObservation−FPKMAverage)/FPKMSD (FPKMObservation−FPKM value obtain through sequencing; FPKMAverage−average of all FPKM values in all samples for a certain gene; FPKMSD−standard deviation of FPKM values for a certain gene). Heat maps were generated using Microsoft Excel conditional formatting.

Comparing epigenetic changes in different cell lines: To compare drug-induced epigenetic changes across multiple glioblastoma cell lines, 101A, 217M, GBM2 and PBT24 cells were plated at 4000 cells/well and treated with compounds for 24 hours. Activity level was calculated as above. Pearson coefficient and significance of correlation for activity levels in each pair of cell lines were calculated using the Excel add-on program xlstat (Base, v19.06).

Correlation of transcriptomic and image-based profiles: Euclidean distances were calculated using either transcriptomic data (FPKM) or texture features. Pearson's correlation coefficient (R) was transformed to a t-value using the formula (t=R×SQRT(N−2)/SQRT(1−R2) where N is the number of samples, R is Pearson correlation coefficient; the p-value was calculated using Excel t.dist.2t(t) function. For compound prioritization, Euclidean distance between the compound treated and serum- or Bmp4-treated GBM2 cells was calculated based on either FPKM) or image features. The average distance for both serum and Bmp4 treatments was normalized to the average distance of untreated cells to serum and Bmp4.

Sensitization to radiation or TMZ: Cells were plated at 1500 cells/well in 384-well optical bottom assay plates (PerkinElmer). Two sets of the experiment were prepared; DMSO (0.1%) was used for negative controls at 48 DMSO replicates per plate; 3 replicates (wells) were treated per compound. Cells in both sets were pre-treated with epigenetic compounds for 2 days prior to cytotoxic treatment. Cytotoxic treatment, either 200 uM temozolomide (TMZ, Sigma) or 1 Gy x-ray radiation (RS2000; RAD Source) was carried out for 4 days on single set (‘treatment set’); for TMZ treatment, DMSO control was given to the second set. A single radiation dose was given at day 3; TMZ was given twice at days 3 and 5 of the experiment. Cells were fixed, stained with DAPI, and scored using an automated microscope (Celigo; Nexcelom Bioscience). For each compound, fold change in cell number was calculated for both the “treatment set” (Drug+Cytotox) and the “control set” (Drug), compared to DMSO-treated wells in the control set. The effect of radiation or TMZ alone was calculated as fold reduction of DMSO-treated wells in the treatment set compared to DMSO-treated wells in the control set (Cytotox). The coefficient of drug interaction (CDI) was calculated as (Drug+Cytotox)/(Drug)×(Cytotox). For conformation experiments, the same regiment and CDI calculations were carried out on SK262, 101A, 217M, 454M, and PBT24 glioblastoma cell lines; PARPi and BETi were used at same concentration as the initial screen on GBM2.

Prestwick Chemical Library screen using H3K27me3 and H3K27ac: GBM2 cells were plated at 2000 cells/well and exposed to Prestwick compounds (3 μM) for 3 days in 384-well optical bottom assay plates (PerkinElmer). Cells were then fixed and stained with rabbit polyclonal anti-H3K27ac and mouse monoclonal anti-H3K27me3 antibodies followed by AlexaFluor-488- or AlexaFluor-555-conjugated secondary antibodies. Positive controls contained BMP4 (100 ng/ml) and serum (10%); negative controls contained DMSO (0.1%). DNA was counterstained with Hoechst. Images were acquired using Perkin Elmer Opera® QEHS. MIEL analysis was conducted as described above.

Development of the MIEL Platform

A novel phenotypic screening platform, MIEL, is developed, which interrogates the epigenetic landscape at both population and single cell levels using image derived features and machine learning. MIEL takes advantage of epigenetic marks such as histone methylation and acetylation, which are always present in eukaryotic nuclei and can be revealed by immunostaining. MIEL analyzes the immunolabeling patterns of epigenetic marks using conventional image analysis methods for nuclei segmentation, feature extraction, and previously described machine-learning algorithms (FIG. 21A of International Publication No. WO 2022/120219A1, which is incorporated by reference herein in its entirety). Primarily, four histone modifications were utilized: H3K27me3 and H3K9me3, which are associated with condensed (closed) facultative and constitutive heterochromatin, respectively; H3K27ac, associated with transcriptionally active (open) areas of chromatin, especially at promoter and enhancer regions; and H3K4me1, associated with enhancers and other chromatin regions (FIG. 21A of International Publication No. WO 2022/120219A1, which is incorporated by reference herein in its entirety). To focus on the intrinsic pattern of epigenetic marks, only texture-associated features were used (e.g., Haralick's texture features, threshold adjacency statistics, and radial features) for multivariate analysis. Previous studies have successfully employed similar features for cell painting techniques combined with multivariate analyses to accurately classify subcellular localization of proteins, cellular subpopulations, and drug mechanisms of action.

Three main methods of data visualization and analysis were employed: To visualize similarity between multiple cell populations, the multivariate centroids were calculated for each cell population and the Euclidean distance between all populations. To reduce data dimensionality and present as a 2D scatter plot (termed distance map), multidimensional scaling was used (MDS; FIG. 21A of International Publication No. WO 2022/120219A1, which is incorporated by reference herein in its entirety). To classify multiple cell populations, quadratic discriminant analysis of multivariate centroids was employed, while single cells across cell populations were classified using a Support Vector Machine (SVM; FIG. 21A of International Publication No. WO 2022/120219A1, which is incorporated by reference herein in its entirety).

The most commonly used cellular assays for epigenetic drug discovery are lysis and ELISA, such as AlphaLISA (PerkinElmer). Imaging-based alternatives rely on staining for relevant histone modification and monitoring changes in average fluorescent intensity. Using MIEL, a library of 222 epigenetically active compounds were screened, many with known targets among epigenetic writers, erasers, or readers (SBP epigenetic library, FIGS. 26A-B of International Publication No. WO 2022/120219A1, which is incorporated by reference herein in its entirety). MIEL's ability to (1) detect active compounds; (2) group drugs by function and identify off-target effects; (3) be robust across cell lines and drug concentrations; (4) rank active drugs and derive information regarding drug mechanism of action was examined.

MIEL Improves Detection of Epigenetically Active Drugs

To determine how well MIEL could detect active compounds and compare them against other intensity-based methods, primary-derived TPCs (GBM2 cell line) were treated with epigenetically active drugs for 24 hours (10 μM, triplicates). Treated cells were immunolabeled for multiple histone modifications expected to exhibit alterations following drug treatment (H3K9me3, H3K27me3, H3K27ac, and H3K4me1). Image analysis, including nuclei segmentation and features extraction, was conducted on an Acapella 2.6 (PerkinElmer). Phenotypic profiles were generated for each compound or control-treated (DMSO) treated wells. These are vectors were composed of 1048 (262 features per modification×4 modifications) texture features derived from the staining of each modified histone modification and representing the average value for each feature across all stained cells in each cell population (drug or DMSO). When treatment reduced cell count to under 50 imaged nuclei per well, the compound was deemed toxic and excluded from analysis. Following feature normalization by z-score, the Euclidean distance between vectors of the compounds and DMSO− treated cells was calculated. These distances were then normalized (z-score) to the average distance between DMSO replicates and the standard deviation of these distances. Compounds with a distance z-score of greater than 3 were defined as active. This analysis identified 122 compounds that induced significant epigenetic changes. Active compounds were not uniformly distributed across all functional drug categories. Rather, 10 categories were identified in which 50% of the drugs were identified as active and nontoxic and 13 categories in which 25% or less fewer of the drugs induced detectable epigenetic alterations following a 24-hour treatment (FIG. 21B of International Publication No. WO 2022/120219A1, which is incorporated by reference herein in its entirety).

To compare MIEL with current thresholding methods, the calculation was repeated using mean fluorescence intensity for all histone modifications by normalizing (z-score) each drug against DMSO; active compounds were defined as compounds for which z-scored intensity for at least one of the histone modifications was greater than 3 or less than −3. As a result, 94 active compounds were identified, which were distributed across functional categories similarly to MIEL-identified compounds (FIG. 21B of International Publication No. WO 2022/120219A1, which is incorporated by reference herein in its entirety). For each functional category, the number of compounds identified as active using thresholding was fewer than the number identified using MIEL (FIG. 21B of International Publication No. WO 2022/120219A1, which is incorporated by reference herein in its entirety), demonstrating MIEL's increased detection sensitivity over standard thresholding.

To determine the contribution of individual histone modifications, both MIEL and thresholding analyses were repeated individually for each of the 4 modifications. Using MIEL-based analysis, a single modification yielded similar detection rates to the combination of modifications across most functional categories (FIG. 27A of International Publication No. WO 2022/120219A1, which is incorporated by reference herein in its entirety). Using intensity-based analysis, individual modifications yielded lower detection rates compared to the combination of modifications and displayed equal or reduced detection rates when compared to MIEL in all categories and modifications (FIG. 27A of International Publication No. WO 2022/120219A1, which is incorporated by reference herein in its entirety). Of note, 3 of the 4 modifications used for MIEL analysis showed similar detection rates across most of the functional categories. However, detection rates of modified H3K27me3 were consistently reduced across the most active categories (FIG. 27A of International Publication No. WO 2022/120219A1, which is incorporated by reference herein in its entirety) except for EZH1/2 inhibitors, possibly due to the role these enzymes play in regulating this posttranslational modification. To further compare MIEL and thresholding, the magnitude of epigenetic alterations induced by active compounds was estimated. The fold increase in distance from DMSO (normalized to average distance between DMSO replicates) was calculated, as well as the fold change in fluorescence intensity for active compounds in each category. In all categories, MIEL showed an increased effect (FIG. 27B of International Publication No. WO 2022/120219A1, which is incorporated by reference herein in its entirety).

These results demonstrate that, across all tested epigenetic modifications, detecting epigenetically active compounds using high content imaging was markedly improved by implementing MIEL compared to current image-based thresholding methods.

MIEL Suggests Functional Groups and Identifies the Off-Target Effects

One key advantage of phenotypic profiling methods like MIEL is the ability to classify compounds by function and identify its nonspecific effects by comparing with profiles of well-defined controls. To assess whether MIEL could correctly group compounds by function, discriminant analysis (DA) was applied to all active, nontoxic compounds from categories that had at least 3 such compounds (85 compounds; 7 categories and DMSO). Two replicates from each drug and 38 DMSO replicates were used as a training set for a quadratic DA, using all texture features derived from images of the four histone modifications (features displaying multicollinearity were reduced). The third replicate for each compound, as well as 10 DMSO replicates, were used as a test set to validate the model. Results showed that MIEL separated multiple categories of epigenetically active drugs with an average accuracy of 91.4% (FIGS. 21C-21D of International Publication No. WO 2022/120219A1, which is incorporated by reference herein in its entirety). Although many of the epigenetically active compounds induced alterations in average fluorescence (FIG. 27B of International Publication No. WO 2022/120219A1, which is incorporated by reference herein in its entirety), a DA utilizing intensity measurements from all 4 channels was ineffective at separating the various categories and yielded only 51.6% average accuracy (FIG. 28A of International Publication No. WO 2022/120219A1, which is incorporated by reference herein in its entirety). To test whether modification textures of individual histones contained sufficient information to distinguish between the various drug classes, DA was performed using features derived from each modification. Although this degraded MIEL's ability to separate compound subclasses, which affected similar changes in histone modification such as Class I and Pan HDAC inhibitors, MIEL was still able to separate major categories, such as histone phosphorylation and deacetylation (FIG. 28B of International Publication No. WO 2022/120219A1, which is incorporated by reference herein in its entirety).

Of note, the compound library used in this study included Pan HDAC inhibitors (HDACi), Class I HDACi, and Class I HDACi, known to also target HDAC6. HDAC inhibitors targeting both Class I and HDAC 6 displayed the same profile as Pan HDAC, and DA showed the two categories to be undistinguishable. This was likely due to the high expression of HDAC Class I and HDAC 6 and low expression of other HDACs in GBM2 glioblastoma line (FIGS. 29A-29C of International Publication No. WO 2022/120219A1, which is incorporated by reference herein in its entirety).

Of the 85 compounds tested, 7 (8.2%) were identified as active but were misclassified by MIEL. One of these was valproic acid, a commonly used anticonvulsant, which also functions as a Pan HDAC inhibitor at high concentrations. Though valproic acid is expected to inhibit HDACs only at high concentrations (>1.2 mM), a short 24-hour treatment induced detectable epigenetic changes even at low concentrations (<30 μM). However, when quantified H3K27ac and H3K27me3 immunofluorescence intensity at these concentrations, no increase in histone acetylation or decrease in histone methylation similar to other Pan HDAC inhibitors (TSA, SAHA; FIG. 30A of International Publication No. WO 2022/120219A1, which is incorporated by reference herein in its entirety) was seen. To test, whether observed epigenetic changes resulted in corresponding transcriptomic alterations, RNA from GBM2 cells treated with either DMSO, TSA, SAHA or valproic acid (15 μM) for 24 hours was sequenced and all genes altered by at least one of the drugs (as compared to DMSO; 118 genes) were identified. The Pan HDAC inhibitors induced similar transcriptomic changes; these were not reflected in the transcriptomic profile of valproic acid-treated cells (FIG. 30B of International Publication No. WO 2022/120219A1, which is incorporated by reference herein in its entirety). To test whether MIEL profiles reflected global drug-induced transcriptomic profiles, FPKM values for all expressed genes (FPKM>1 in at least one cell population) were used to calculate the Euclidean distance between all 4 cell populations. FPKM-based distances were then correlated to image texture feature-based distances, which yielded a high and significant correlation between these metrics (R=0.91, pv<0.05; FIG. 30C of International Publication No. WO 2022/120219A1, which is incorporated by reference herein in its entirety). These results demonstrate a unique ability of the MIEL approach to identify epigenetically active compounds, to accurately categorize them according to their molecular mechanism of action, and to detect off-target effects of compounds with known mechanism of action.

Unbiased Detection of Drug Concentration Effect on Cellular Epigenetic State.

As drugs vary in potency, predicting the function of unknown drugs relies on generating functional category-specific profiles that remain valid over a range of activity levels. To determine whether MIEL could correctly identify the functional category of drugs with different potencies, GBM2 cells were treated with drugs from several active categories at a range of concentrations (0.1, 0.3, 1, 3, 10 μM) and DA aimed at separating the different concentrations in each class was conducted. It was found that for most drug categories (inhibitors of: Aurora, JAK, SIRT and EZH1/2), DA yielded low-average accuracy (FIG. 21A of International Publication No. WO 2022/120219A1, which is incorporated by reference herein in its entirety—Aurora kinase: 43.3%; FIG. 31A of International Publication No. WO 2022/120219A1, which is incorporated by reference herein in its entirety—EZH1/2:62.5%, SIRT:46.2%, JAK: 37.2), indicating similar MIEL profiles across all tested drug concentrations. However, Pan HDAC and HDAC Class I inhibitors displayed progressive profile changes, allowing DA to separate the different concentrations at higher accuracy (FIG. 22A of International Publication No. WO 2022/120219A1, which is incorporated by reference herein in its entirety—HDAC Pan: 80.9%; FIG. 31A of International Publication No. WO 2022/120219A1, which is incorporated by reference herein in its entirety—HDAC Class I: 82.2%).

In addition to their on-target effect, the drugs may induce epigenetic alterations through toxicity and stress. To estimate the impact of toxicity on changes to drug-induced profiles and its contribution to drug misclassification across a range of concentrations, z-scored distance was plotted from DMSO (effect size) against z-scored nuclei count (a proxy for cytotoxicity) for GBM2 cells treated at a range of drug concentrations (e.g., 0.1, 0.3, 1, 3, 10 μM). This demonstrated that some compound classes, such as Aurora and JAK inhibitors, induce epigenetic alterations only in concentrations where cell count is significantly reduced, whether through toxicity or direct effect on proliferation (FIG. 22B of International Publication No. WO 2022/120219A1, which is incorporated by reference herein in its entirety—dark blue and pink respectively), while other compounds, such as HDAC inhibitors, characteristically have a concentration range where epigenetic alterations are not accompanied by reduced cell counts (FIG. 22B of International Publication No. WO 2022/120219A1, which is incorporated by reference herein in its entirety—green and yellow). Interestingly, both SIRT and EZH1/2 (FIG. 22B of International Publication No. WO 2022/120219A1, which is incorporated by reference herein in its entirety—light-blue and red, respectively) inhibitors affected significant epigenetic changes without inducing significant changes in cell count.

These results indicated the MIEL platform is ideally positioned to analyze dose-dependent effects from drug treatment. In particular, this data suggests that low (0.1 uM) and high (10 uM) concentration of HDAC inhibitors resulted in distinct and separable epigenetic landscapes, suggesting potentially distinct chromatin/gene expression profiles and divergent biological outcomes when using a low vs high concentration of such compounds.

MIEL Profiles are Coherent Across Multiple Cell Lines

Testing candidate drugs in multiple cell lines can help gauge their inclusivity and identify tumor subtypes that do not respond to a specific drug or drug class. To test whether MIEL readouts were coherent across multiple glioblastoma TPCs, 4 cell lines were treated with a subset of drugs from the epigenetic library (57 drugs), derived phenotypic profiles, and calculated their effect size (z-scored Euclidean distance from DMSO replicates. This revealed a significant positive correlation between all 4 cell lines pointing to the similarities in their drug sensitivity profiles and demonstrating the robustness of the MIEL read out (FIGS. 22C-D of International Publication No. WO 2022/120219A1, which is incorporated by reference herein in its entirety). To assess the ability of MIEL to group compounds by function across multiple cell lines, DA was employed to classify DMSO and drug treated TPCs across these 4 GBM lines. In this way, cells treated with drugs modulating distinct functions could be accurately separated, such as EZH1/2 or SIRT inhibitors (5 and 3 compounds respectively; mean accuracy 100%; FIG. 22E of International Publication No. WO 2022/120219A1, which is incorporated by reference herein in its entirety). These results demonstrate MIEL's ability to correctly categorize by function drugs with varying degrees of potency across multiple cells lines.

Finally, although individual drug activity correlated well across cells lines, the magnitude of the effect for some classes of drugs was highly correlated to the gene expression levels of the target. For example, SIRT inhibition was significantly more effective in lines showing reduced Sirt1 expression (the main SIRT to deacetylate histone 3; n=4 compounds, p<0.02; FIG. 31C of International Publication No. WO 2022/120219A1, which is incorporated by reference herein in its entirety), and there was a significant inverse correlation between Sirt1 expression and the effect size (R=−0.87; FIG. 31C of International Publication No. WO 2022/120219A1, which is incorporated by reference herein in its entirety). In sum, the MIEL assay was both sensitive and robust across multiple primary human glioblastoma cells lines, which further underscores its ability to detect the differences in gene expression and to provide a cumulative measurement of the effect of each compound on cellular epigenetic landscape.

MIEL Helps Uncover the Mechanism of BET Inhibitors Synergy with TMZ and Ranks their Activity

MIEL analysis demonstrated that the magnitude of drug-induced profile change, as measured by the distance from DMSO controls, vary between individual drugs within each drug class (FIG. 32A of International Publication No. WO 2022/120219A1, which is incorporated by reference herein in its entirety). To test whether these differences are biologically meaningful, MIEL-based activity of epigenetic drugs which are often designed to work in combination was correlated with other treatments. One common approach is to use epigenetic drugs to sensitize tumor cells to a standard of care in cytotoxic treatment, such as radiation and temozolomide (TMZ), which are used to treat glioblastoma. To identify drug classes that sensitize glioblastoma TPCs to cytotoxic therapy, GBM2 cells were treated with epigenetic drugs for 2 days prior to radiation or TMZ. Cytotoxic treatment was carried out for 4 days at levels that induced a 50% reduction in cell numbers (1 Gy radiation or 200 uM TMZ; FIG. 23A of International Publication No. WO 2022/120219A1, which is incorporated by reference herein in its entirety). At the end of the treatment (day 6), cells were counted, and a combined drug index (CDI) was calculated. Multiple drugs from both PARP and BET inhibitor (PARPi and BETi) were identified that could sensitize cells to TMZ (FIG. 23B of International Publication No. WO 2022/120219A1, which is incorporated by reference herein in its entirety, left panel).

PARPi have been extensively studied in this context and have been shown to function through multiple nonepigenetic mechanisms such as PARP trapping. Consistent with this, most PARPi did not induce detectable epigenetic changes using MIEL (FIG. 23D and FIG. 32B of International Publication No. WO 2022/120219A1, which is incorporated by reference herein in its entirety), and no correlation was found between the magnitude of epigenetic changes as measured by MIEL and CDI (FIG. 23D of International Publication No. WO 2022/120219A1, which is incorporated by reference herein in its entirety—bottom panel). To date, only a single report utilizing the BETi OTX015 has pointed to synergy with TMZ, prompting the validation of this finding in 6 additional glioblastoma lines. In 3 lines, BETi increased the TMZ effectiveness (average CDI. 454M 0.76±0.28, PBT24 0.78±0.12 and GBM2 0.51±0.2; Mean±SD; n=11 BETi; FIG. 23C of International Publication No. WO 2022/120219A1, which is incorporated by reference herein in its entirety). In the other 3 lines, the drugs did not synergize and, in many cases, were found to be protective against (CDI>1) TMZ (average CDI. SK262 1.4±0.26, 101A 1.4±0.22 and 217M 1.2±0.21; Mean±SD; n=11 BETi; FIG. 23C of International Publication No. WO 2022/120219A1, which is incorporated by reference herein in its entirety; p− values for all pairwise comparisons). Only a few BETi-induced epigenetic changes occurred during 24-hour initial screening (FIG. 21B of International Publication No. WO 2022/120219A1, which is incorporated by reference herein in its entirety). However, following 6 days of treatment, 6 of 11 BETi induced significant (average z-scored distance from DMSO replicates>3) epigenetic changes in all cell lines (FIG. 23D and FIG. 32B of International Publication No. WO 2022/120219A1, which is incorporated by reference herein in its entirety). In lines displaying TMZ and BETi synergy, the degree of BETi activity, as measured by MIEL, significantly correlated with the degree of synergism (FIG. 23D—top panel of International Publication No. WO 2022/120219A1, which is incorporated by reference herein in its entirety). This demonstrated that for individual compounds, MIEL can predict relative drug activity and suggests an epigenetic component for the mechanism of BETi-TMZ synergy.

O⁶-alkylguanine DNA alkyltransferase (MGMT), which provides the main line of defense against DNA alkylating agents such as TMZ, has been found to be epigenetically silenced through DNA methylation in a large fraction of glioblastoma tumors. To gain a better understanding of the mechanism by which BETi sensitizes glioblastoma TPCs to TMZ treatment, MGMT expression was quantified in the 6 lines tested using qPCR. Analysis showed that while all lines expressed similar BET-TF levels, such as Brd2 (FIG. 23E of International Publication No. WO 2022/120219A1, which is incorporated by reference herein in its entirety), and were thus susceptible to BET inhibitors, only 3 lines displaying BETi-TMZ synergy expressed MGMT (FIG. 23E of International Publication No. WO 2022/120219A1, which is incorporated by reference herein in its entirety). Yet after treating those 3 lines with BETi, MGMT expression was dramatically reduced (FIG. 23F of International Publication No. WO 2022/120219A1, which is incorporated by reference herein in its entirety). Finally, after combining BET inhibitors with the MGMT inhibitor Lomeguatrib, no increase was detected in sensitivity to TMZ above the levels conferred by Lomeguatrib alone (FIG. 23G of International Publication No. WO 2022/120219A1, which is incorporated by reference herein in its entirety). In sum, several BETi synergized with TMZ treatment by reducing MGMT expression. It was determined that the degree of synergism displayed by individual BETi positively correlated with the magnitude of their epigenetic effect as measured using MIEL, suggesting that their mechanism of action involves epigenetic change. In contrast, the activity of PARP inhibitors didn't correlate with MIEL distance, suggesting an alternative mechanism of action unrelated to epigenetic changes.

MIEL Discriminates Between Multiple Cell Fates.

To determine whether MIEL could discriminate between different cell fates, 3 cell types were analyzed: primary human fibroblasts, induced pluripotent stem cells (iPSCs) derived from these fibroblasts, and neural progenitor cells (NPCs) differentiated from the iPSCs. The fibroblasts were isolated from 3 unrelated donors (WT-61, WT-101, WT-126) and used to obtain corresponding iPSC and NPC lines. Cellular identities of the 3 cell types were verified by immune-fluorescence for Sox2 and Oct4 (FIG. 24A of International Publication No. WO 2022/120219A1, which is incorporated by reference herein in its entirety), and MIEL analysis was carried out using data from either H3K4me1 and H3K9me3 or H3K27ac and H3K27me3 staining, with both combinations providing similar results. Multivariate centroids were calculated for each cell population and plotted on a distance map to visualize the relative Euclidean distance between various cell populations. The fibroblasts, iPSCs, and NPCs each segregate to form 3 visually distinct territories (FIGS. 33A-33D of International Publication No. WO 2022/120219A1, which is incorporated by reference herein in its entirety). The 9 lines were separated by cell-fates using DA, which showed an accurate separation of the different cell-fates across all 3 donors (average accuracy 100%; FIG. 24B and FIG. 33E of International Publication No. WO 2022/120219A1, which is incorporated by reference herein in its entirety). A similar analysis performed to separate the different donors showed only low accuracy (average accuracy 55.5%; FIG. 24C and FIG. 33F of International Publication No. WO 2022/120219A1, which is incorporated by reference herein in its entirety). To determine whether it was possible to discriminate between individual cells with different fates, a Support Vector Machine (SVM) classifier was trained on a subset of fibroblasts, iPSCs, and NPCS from the 3 donors. Classification of the test set indicated a high degree of separation between the different fates at a single cell level (FIGS. 33B, 33D of International Publication No. WO 2022/120219A1, which is incorporated by reference herein in its entirety). FIG. 33G of International Publication No. WO 2022/120219A1, which is incorporated by reference herein in its entirety, shows a distance map depicting the relative Euclidean distance between the multiparametric centroids of 3 genetically distinct TCP and DGC lines calculated using texture features derived from images of H3K9me3 and H3K4me1 marks. Additionally, MIEL analysis (using only H3K9me3) was able to discriminate between the main lineages of primary hematopoietic cell types freshly isolated from mouse bone marrow, namely lymphoid, myeloid, and stem/progenitors (FIG. 34 of International Publication No. WO 2022/120219A1, which is incorporated by reference herein in its entirety). However, closely related cell types in each lineage such as hematopoietic stem and progenitor cells were not readily separated (FIG. 34 of International Publication No. WO 2022/120219A1, which is incorporated by reference herein in its entirety).

These results underscore MIEL's ability to discriminate multiple different cell types and differentiation states uniquely based on their single-cell epigenetic landscapes both in cultured and primary cells of human and mouse origin.

MIEL Determines the Signatures of Glioblastoma Stem Cells and Differentiated Glioblastoma.

Most epigenetic drugs are known to directly affect the level histone and DNA modifications, which are the substrates MIEL assay. To test whether MIEL is capable to identify and classify drugs that affect epigenetic landscape indirectly, glioblastoma differentiation paradigm was examined. Although such approach was proposed by several groups, identification of small molecule inducers of glioblastoma differentiation has been challenging.

MIEL's ability was tested to distinguish TPCs and differentiated glioma cells (DGCs), derived from primary human glioblastomas. Three TPC/DGC pairs were derived in parallel from 3 genetically distinct glioblastoma tumor samples (MGG4, MGG6, and MGG8) over a 3-month period using either serum-free FGF/EGF for TPCs or 10% serum for DGCs (10). Visualization using a distance map demonstrated that TPCs and DGCs segregate to form two visually distinct territories (FIG. 33G of International Publication No. WO 2022/120219A1, which is incorporated by reference herein in its entirety) and were separated with high accuracy using DA (mean accuracy 100%; FIG. 24D of International Publication No. WO 2022/120219A1, which is incorporated by reference herein in its entirety). SVM-based pairwise classification of single cells distinguished TPCs from their corresponding DGC lines with an average accuracy of 83%, using any of the 4 epigenetic marks tested (H3K27me3, H3K9me3, H3K27ac, and H3K4me1; FIG. 24E of International Publication No. WO 2022/120219A1, which is incorporated by reference herein in its entirety). An SVM classifier derived from images of the MGG4 TPC/DGC pair separated all 3 TPC/DGC pairs with 88% average accuracy, providing proof of principle for the derivation of a signature for nontumorigenic cells obtained following serum differentiation of primary glioblastoma cells (FIG. 24F of International Publication No. WO 2022/120219A1, which is incorporated by reference herein in its entirety).

These findings suggest that MIEL can readily distinguish undifferentiated TPCs from differentiated DGCs based on multiparametric signatures of these glioblastoma cells using only the patterns of universal epigenetic marks. Of note, such signatures could only be obtained using simultaneous assessment of dozens of transcripts by averaging thousands of cells.

Short-Term Treatment with Serum or Bmp4 Initiates TPC Differentiation

For the purpose of establishing a screening protocol, whether short serum or Bmp4 treatment is sufficient to induce a differentiation-like phenotype in TPCs was tested. Several glioblastoma cell lines were treated for 3 days with either serum or Bmp4, then expression of core transcription factors was quantified to determine the TPC transcriptomic program. Immunostaining revealed that the 4 transcription factors Sox2, Sall2, Brn2 and Olig2 were downregulated by both serum and Bmp4 in a cell line-dependent manner (FIG. 35A of International Publication No. WO 2022/120219A1, which is incorporated by reference herein in its entirety). RNAseq analysis of serum- and Bmp4-treated GBM2 cells revealed that 3 days of treatment reduced (vs untreated cells) expression of most genes previously found to constitute the transcriptomic stemness signature (FIG. 35B of International Publication No. WO 2022/120219A1, which is incorporated by reference herein in its entirety). Additionally, both serum and Bmp4 were found to attenuate TCP growth rate (FIG. 35C of International Publication No. WO 2022/120219A1, which is incorporated by reference herein in its entirety). To identify the cellular processes altered by these treatments, differential expression analysis was conducted. Expression of 4852 genes was significantly altered (p<0.01 and −1.5<Fold Change>1.5) by either serum or Bmp4. Gene Ontology (GO) analysis of these altered genes indicated enrichment in multiple GO categories consistent with initiation of TPC differentiation; these include cell cycle, cellular morphogenesis associated with differentiation, differentiation in neuronal lineages, histone modification, and chromatin organization (FIG. 36 of International Publication No. WO 2022/120219A1, which is incorporated by reference herein in its entirety).

These results demonstrate that a 3-day treatment with either serum or Bmp4 is sufficient to induce transcriptomic changes characteristic of TPC differentiation. Indeed, distinct expression changes were observed, including differences in expression of genes regulating chromatin organization and histone modifications (FIGS. 37A-37B of International Publication No. WO 2022/120219A1, which is incorporated by reference herein in its entirety) between serum- and Bmp4-induced glioblastoma differentiation.

MIEL Detects Epigenetic Changes Following Short-Term Serum or Bmp4 Treatment.

Four genetically distinct glioblastoma lines were treated with serum or BMP4, then conducted MIEL analysis using either H3K9me3 and H3K4me1 or H3K27ac and H3K27me3 to detect TPC differentiation. Discriminant analysis allowed high accuracy separation of these treatments across all cell lines using both histone modification combinations (mean accuracy 100%; FIG. 24H and FIG. 37C of International Publication No. WO 2022/120219A1, which is incorporated by reference herein in its entirety).

The global gene expression profile represents a gold standard for defining the cellular state. To test whether MIEL reliably reports the epigenetic changes associated with serum and Bmp4 treatments, a correlation between MIEL-based and global gene expression-based metrics was conducted. Untreated and 3 days serum- or Bmp4-treated GBM2 TPCs were sequenced. All genes with FPKM>1 in at least one cell population were used to calculate the Euclidean distance matrix between all cell populations. FPKM-based distances were then correlated to image texture feature-based distances. The resulting Pearson correlation coefficient of R=0.93 (p<0.001) suggests a high correlation between these two metrics (FIGS. 24J-24K of International Publication No. WO 2022/120219A1, which is incorporated by reference herein in its entirety), demonstrating that MIEL is capable of distinguishing closely related glioblastoma differentiation routes induced by serum or BMP and validating the robustness of the MIEL approach for analyzing glioblastoma differentiation.

MIEL Successfully Prioritizes Small Molecules Inducing TPCs Differentiation.

The Prestwick compound library (˜1200 compounds) was screened using MIEL to identify compounds inducing glioblastoma TPC differentiation based on the differentiation signatures obtained with serum/Bmp4 treatments. GBM2 TPCs were treated for 3 days with Prestwick compounds at 3 μM fixed, then immunolabeled for H3K27ac and H3K27me3. GBM2 cells treated with DMSO, serum, BMP4, or compound were compared within the same plate (to avoid imaging artifacts and normalization issues).

To identify epigenetically active compounds, the Euclidean distance to the DMSO center for each DMSO replicate and Prestwick compound was calculated. Distances were z-scored, and active compounds were defined as compounds for which z-scored distance was greater than 3. Compounds with less than 50 cells imaged were considered toxic and excluded from analysis. Following analysis, MIEL identified 144 active compounds. To identify compounds inducing epigenetic changes reminiscent of serum-BMP4-induced differentiation, quadratic DA was used to build a model separating untreated, serum-treated, and Bmp4-treated cells and classified all 144 active compounds to these categories (FIGS. 25A-25B of International Publication No. WO 2022/120219A1, which is incorporated by reference herein in its entirety). A total 31 compounds were classified as similar to either serum or Bmp4 (i.e., differentiated). Of these, 20 compounds belonged to 1 of the following 4 categories: Na/K-ATPase inhibitors of the digoxin family, molecules that disrupt microtubule formation or stability, topoisomerase inhibitors, or nucleotide analogues that disrupt DNA synthesis (FIG. 25B of International Publication No. WO 2022/120219A1, which is incorporated by reference herein in its entirety). To further narrow down the list of candidates, pairwise SVM classification of DMSO- and either serum- or BMP4-treated cells was conducted, and compounds were selected that induced at least 50% of the cells to be classified as either serum- or BMP4-treated. The Euclidean distance between candidate compounds and serum- and BMP4-treated cells was calculated; compounds were selected where the distance to one or both treatments was less than the distance between DMSO and that treatment. Of the 20 candidate compounds identified, 15 belonged to 1 of the 4 categories mentioned above (FIG. 38A of International Publication No. WO 2022/120219A1, which is incorporated by reference herein in its entirety).

From the 15 candidate compounds, 2 top compounds were chosen from each of the four categories (8 total) for further analysis. GBM2 cells were treated for 3 days with DMSO, serum, Bmp4 or candidate compounds at 0.3, 1, or 3 μM, fixed, then immunostained for H3K27ac and H3K27me3. Using pairwise SVM-based classifications of untreated cells and either serum- or Bmp4-treated cells identified for each of the 8 compounds, the lowest concentration at which at least 50% of the cells were classified as treated (FIG. 38B of International Publication No. WO 2022/120219A1, which is incorporated by reference herein in its entirety) and used those concentrations for all subsequent experiments.

Because most of these compounds are known for their cytotoxic effects, the growth rates of drug-treated glioblastoma cells were verified. With the exception of digoxin, which was cytostatic, drug treatment resulted in growth rates comparable with those induced by serum or BMP4 (FIG. 39A of International Publication No. WO 2022/120219A1, which is incorporated by reference herein in its entirety). Immunofluorescence was used to test for expression of core TPC transcription factors (Sox2, Sall2, Brn2 and Olig2). Except for trifluridine, all compounds induced statistically significant reductions in Sox2; digoxin and digitoxigenin also induced a significant reduction of Sall2 and Brn2; olig2 expression was unaltered by any treatment (FIG. 39B of International Publication No. WO 2022/120219A1, which is incorporated by reference herein in its entirety).

Next, it was investigated whether the compounds identified using MIEL can induce transcriptomic changes similar to serum and Bmp4 treatment and quantified the ability of MIEL to predict compounds best at mimicking these treatments. GBM2 cells were treated with DMSO, serum, Bmp4, or each of the eight candidate compounds; after 3 days, RNA was extracted and sequenced. Transcriptomic profiles of the eight compounds were ranked according to average Euclidean distance (based on FPKM values for all expressed genes) from serum- or BMP4-treated cells. To safeguard against potential artefacts of cytotoxicity, gene expression-based ranking was compared with measured cellular growth rates from drug treatments and found no positive correlation (FIG. 39C of International Publication No. WO 2022/120219A1, which is incorporated by reference herein in its entirety). When Sox2 expression levels under all treatment conditions were compared to determine whether the transcription factor can identify drugs that best mimic serum or BMP4, no positive correlation was found between either expression levels or transcriptomic-based rankings (FIG. 39D of International Publication No. WO 2022/120219A1, which is incorporated by reference herein in its entirety), suggesting that Sox2 levels alone are insufficient to stratify the compounds. Finally, to compare MIEL-based signatures to the transcriptomic profile, MIEL readouts of cells treated with the eight drugs were ranked according to average Euclidean distance from serum- or Bmp4-treated cells (calculated using texture features derived from images of H3K27ac, H3K27me3, H3K9me3, and H3K4me1). Comparison of the MIEL-based metric with the gene expression-based metric revealed a high degree of positive correlation between MIEL- and gene expression-based rankings (Pearson correlation coefficient R=0.92, p<0.001; FIG. 25C of International Publication No. WO 2022/120219A1, which is incorporated by reference herein in its entirety). To further visualize these results, heat maps were constructed to depict fold change in gene expression associated with several GO terms enriched by serum and Bmp4. The top candidate, etoposide, altered expression of a large portion of genes in similar fashion to that of serum and BMP4; in contrast, the lowest-ranking candidate, digoxin, induced changes in gene expression, which were rather different from serum and BMP4 (FIG. 25D of International Publication No. WO 2022/120219A1, which is incorporated by reference herein in its entirety).

Taken together, the above results suggest unique ability of MIEL to identify molecules that shift epigenetic signature of glioblastoma TPCs towards DGCs. MIEL is capable of ranking such molecules according to their change-inducing potency and that ranking robustly correlate with global expression-based readouts of glioblastoma differentiation.

The pipeline developed herein employs information extracted from immunofluorescence images of specific histone modifications and is geared towards drug discovery and high-throughput screening. MIEL markedly improves detection compared to conventional intensity-based thresholding approaches and enables functional categorization of such compounds. Also, MIEL readouts are coherent across multiple compound concentrations and cell lines and can provide information regarding drug activity levels and their mechanism of action. MIEL also could robustly report cellular fate and provide proof of concept for identifying and prioritizing drugs inducing differentiation of glioblastoma TPCs.

One objective of this example is to estimate the resolution of separation between categories of compounds with similar functions. It was found that a single histone modification was sufficient to separate highly distinct classes. Separating similar classes (e.g., Aurora and JAK inhibitors, which affect histone phosphorylation, or Pan and Class I HADCs, which affect histone acetylation) required staining for at least one additional histone modification. Despite their many advantages, cellular assays, including high-content assays, are often used as secondary screens for epigenetic drugs due to multiplicity of enzyme family members and an inability to determine direct enzymatic activity. Consequently, MIEL's ability to separate closely related functional categories on top of other advantages make this profiling approach an attractive alternative for primary screens.

Phenotypic profiling methods have been previously used to identify genotype-specific drug responses by comparing profiles across multiple isogenic lines. In this example, it is shown that biologic activity (i.e., serum and Bmp4) that induces glioblastoma differentiation, as well as that of 57 epigenetic compounds, was significantly correlated across four different primary glioblastoma lines. Also, it is shown that variation in activity levels correlated with target expression levels and that the various categories can be distinguished across cell lines. Together, these suggest that MIEL could be used to identify cell lines showing an aberrant reaction to selected drugs and, therefore, aid in identifying optimal treatments for individual patients. Similar applications have previously been used to tailor specific kinase inhibitors to patients with chronic lymphocytic leukemia (CLL) who display venetoclax resistance.

Given the limited success of cytotoxic drugs to treat glioblastoma, alternative approaches were used: (1) epigenetic drugs aimed at sensitizing glioblastoma TPCs to such treatments, and (2) inducing glioblastoma differentiation. The data demonstrated MIEL's ability to rank candidate drug activity to correctly predict the best candidates for achieving the desired effect. The importance of this is highlighted in large (hundreds of thousands of compounds) chemical library screens, which typically identify many possible hits needing to be reduced and confirmed in secondary screens.

The results shown in this example uncovered a strong correlation between BET inhibitor activity (measured by MIEL) and its ability to synergize with TMZ and reveal a previously unknown role for BET inhibitors in reducing MGMT expression. Previous studies have demonstrated upregulation of several BET transcription factors in glioblastomas, and multiple pre-clinical studies have investigated the potential of BET inhibition as a single drug treatment for glioblastoma. However, while clinical trials with the BET inhibitor OTX015 demonstrated low toxicity at doses achieving biologically active levels, no detectable clinical benefits were found. This prompted approaches using drug combinatorial treatments such as combining HDACi and BETi. However, the mechanism by which BETi induces increased TMZ has not been described. Recently, a distal enhancer regulating MGMT expression was identified. Activation of this enhancer by targeting a Cas9-p300 fusion to its genomic locus increased MGMT expression while deletion of this enhancer reduced MGMT expression. As BET transcription factors bind elevated H3K27ac levels found in enhancers, this may be a possible mechanism for BETi-induced reduction of MGMT expression, which in turn result in increased sensitivity to the DNA alkylating agent TMZ.

Silencing the MGMT gene through promoter methylation has long been known to make TMZ treatment more responsive and to improve prognosis in patients with glioblastoma. Yet, clinical trials that combine TMZ and MGMT inhibitors have not improved therapeutic outcomes in such patients, possibly due to the 50% reduction in dose of TMZ, which is required to avoid hematologic toxicity. Thus, BETi offers an attractive line of research, though further studies are needed to determine whether the elevated sensitivity of glioblastoma to BETi, and its ability to reduce MGMT expression, thus synergizing with TMZ, could be exploited to improve patient outcome. Thus, MIEL approach is well positioned to systematically analyze and identify epigenetically active compounds, then provide critical initial information for their mechanism of action.

In this example, the Prestwick chemical library of 1200 approved drugs was analyzed to validate MIEL's ability to select and prioritize small molecules, which mimic the effect of serum and BMP4, using global gene expression profiling. Surprisingly, it is observed that the degree of reduction in endogenous SOX2 protein levels following drug treatment did not correlate with the degree of differentiation assessed by global gene expression; in contrast, MIEL-based metrics did correlate. This result, taken together with MIEL's ability to distinguish multiple cells types (iPSCs, NPCs, fibroblasts, hematopoietic lineages) across several genetic backgrounds, suggests that the MIEL approach does not only readily identify compounds by inducing desired changes in cell fate but, specifically, can be a cost-effective tool for prioritizing hundreds of thousands of compounds during the primary screenings.

### Example 9: Microscopic Imaging of Biological Age (miBioAge), Sometimes Also Referred as MIEL-CLOCK

Microscopic Imaging of Biological Age (miBioAge), sometimes referred as MIEL-CLOCK, identifies distinct epigenetic signatures of different primary cells (e.g. hepatocytes, splenocytes, fibroblasts) freshly isolated from the organism (with/without subsequent culturing in the dish) at single cell level and at different chronological ages. In this example, it was tested whether MIEL can be used to compare and distinguish epigenetic signatures of different cell types at different old and young ages (e.g., old=22-24 month, young=4-6 months) using machine learning algorithms (e.g., Support Vector Machine/Support Vector Regression or quadratic discriminant analysis) based on bumpiness features (e.g., Haralick features). As shown in FIGS. 40A-40E of International Publication No. WO 2022/120219A1, which is incorporated by reference herein in its entirety, Hepatocytes (FIGS. 40A-40C of International Publication No. WO 2022/120219A1, which is incorporated by reference herein in its entirety), Spleen cells (mixed population FIGS. 40D-40F of International Publication No. WO 2022/120219A1, which is incorporated by reference herein in its entirety) or fibroblasts (7 day cultured, FIGS. 40G-40H of International Publication No. WO 2022/120219A1, which is incorporated by reference herein in its entirety) were derived from either 5 months or 25 months old mice, and their epigenetic signatures were determined. FIGS. 40A, 40D, and 40G of International Publication No. WO 2022/120219A1, which is incorporated by reference herein in its entirety, show distance maps depicting the relative Euclidean distance following MDS between the multiparametric centroids (replicates) of primary derived hepatocytes (FIG. 40A), spleen cells (FIG. 40D) or fibroblasts (FIG. 40G), calculated from texture feature values derived from images of either H3K9ac and H3K27me3, H4K20me3 and H3K27ac or H3K9me3 and H3K4me1 marks. FIGS. 40B-40C, 40E-40F, and 40H of International Publication No. WO 2022/120219A1, which is incorporated by reference herein in its entirety, show quadratic discriminant analyses using texture features derived from images of hepatocytes (FIGS. 40B-40C of International Publication No. WO 2022/120219A1, which is incorporated by reference herein in its entirety), spleen cells (FIGS. 40E-40F of International Publication No. WO 2022/120219A1, which is incorporated by reference herein in its entirety), or fibroblasts (FIG. 40H of International Publication No. WO 2022/120219A1, which is incorporated by reference herein in its entirety). Cells were stained for either H3K9ac and H3K27me3, H4K20me3 and H3K27ac, H3K9me3 and H3K4me1 marks, H3K9me only, H3K27me3 only, or H4K20me3 only (left FIGS. 40B, 40E, and 40H of International Publication No. WO 2022/120219A1, which is incorporated by reference herein in its entirety; top FIGS. 40C and 40F of International Publication No. WO 2022/120219A1, which is incorporated by reference herein in its entirety). Scatter plot depicts the first discriminant factor for each cell population (replicate) (right FIGS. 40B, 40E, and 40H of International Publication No. WO 2022/120219A1, which is incorporated by reference herein in its entirety; bottom FIGS. 40C and 40F of International Publication No. WO 2022/120219A1, which is incorporated by reference herein in its entirety). Confusion matrices shows results for the validation set and numbers depict the percent of replicates.

To conduct the hepatocyte experiments above, hepatocytes were isolated via a 2-step perfusion method, a first perfusion with isotonic solution (HBSS) to flush away blood and then with a type IV collagenase media to digest the ECM. This was followed by 4 cycles of washes (centrifugation at RCF 50×g), then a 40% Percoll centrifugation step to remove dead hepatocytes resulting in ˜98% pure population of primary hepatocytes. The hepatocytes were seeded on type I collagen treated plates and maintained in culture medium (DMEM, Glut, pen/strep, 10% FBS) overnight. Hepatocytes isolated from 5 months old and 25 months old mice were compared. The experiments were conducted as previously described. Briefly, following labeling with antibodies specific for corresponding epigenetic marks, images were automatically segmented and ˜260 texture features including image moments, Haralick, and Threshold Adjacency Statistics were computed. Values for each cell were exported to Microsoft Excel or MATLAB, using the Excel add-on program Xlstat Multidimensional scaling (MDS) and quadratic discriminant analysis were conducted using the Excel add-on xlstat (Base, v19.06).

Similar studies were carried out with peripheral blood mononuclear cell (PBMC) and CD3+ T cells (FIGS. 40I-40J of International Publication No. WO 2022/120219A1, which is incorporated by reference herein in its entirety) derived from either 5 months, 12 months, 20 months, or 25 months old mice, to determine their epigenetic signatures based on H4K20me3 staining. Quadratic discriminant analysis using texture features derived from respective groups of images using H4K20me antibodies, and confusion matrices showing results for the validation set. Numbers shown in the table represent the percentage of accurate separation. miBioAge has also shown utility for use in human cells, including PBMC (FIG. 40K of International Publication No. WO 2022/120219A1, which is incorporated by reference herein in its entirety). Quadratic discriminant analysis using texture features derived from images obtained using antibodies to H3K9me3, H3K27me3, H4K20me3. Scatter plots of first discriminant factor (H3K9me3 or H4K20me3) are shown on the left in FIGS. 40I-40J of International Publication No. WO 2022/120219A1, which is incorporated by reference herein in its entirety. Large dots with black circles show the distribution of signatures for different mice and ages. Smaller color dots show the distribution of individual measurements for the same mice. Confusion matrices showing results for the validation set; numbers are the percentage of accurate separation.

FIGS. 40L-40M of International Publication No. WO 2022/120219A1, which is incorporated by reference herein in its entirety, show bar graphs of 1) average Euclidean distance between hepatocytes of 5 months or 25 months old animals as calculated from texture feature values derived from images of either H3K9ac, H3K27me3, H4K20me3, H3K27ac, H3K9me3 or H3K4me1 marks (FIG. 40G of International Publication No. WO 2022/120219A1, which is incorporated by reference herein in its entirety), and of 2) average fluorescence intensity measured for hepatocytes of 5 months or 25 months old animals stained for either H3K9ac, H3K27me3, H4K20me3, H3K27ac, H3K9me3 or H3K4me1 marks (FIG. 40H of International Publication No. WO 2022/120219A1, which is incorporated by reference herein in its entirety). This data shows that average Euclidean distances calculated from texture features of any of 6 markers show significant difference between young (5 month) and old (25 month) animals, thus indicates that average Euclidean distances reliably differentiate cells from two different chronological ages.

Using miBioAge, it is observed that the average distance between the centroids from hepatocytes isolated from young mice is smaller than the average distance between the centroids from hepatocytes isolated from old mice. This phenomenon could reflect the increase dispersion of virtually all biological measurements with age. Importantly, miBioAge enables derivation of epigenetic signatures of different cell types at single cell level and at different chronological ages (including human cells) thus representing unique approach to understand biological aging at single cell level. Further, miBioAge enables the use of epigenetic signatures of different cell types at different chronological ages (including human cells) to screen for small molecules that induce epigenetic changes in older cells, which make them similar to the young(er) cells.

MIEL-CLOCK (miBioAge) in Blood Discriminates Against Multiple Age-Groups

To demonstrate the ability of miBioAge determining the biological age of the blood cells or to demonstrate the ability of miBioAge determining the biological age of the cells obtained from the blood tissue, PBMCs from mice were isolated at the indicated ages (FIG. 41A of International Publication No. WO 2022/120219A1, which is incorporated by reference herein in its entirety) and labeled them using H3K9me3 and H3K4me1 markers (FIGS. 41B-41E of International Publication No. WO 2022/120219A1, which is incorporated by reference herein in its entirety), and in some cases, H4K20me3 markers (FIGS. 41F-41G of International Publication No. WO 2022/120219A1, which is incorporated by reference herein in its entirety). Employing classical linear regression analysis implemented in Xlstat package (Base, v19.06) to select features with the best prediction power/correlation with chronological age (FIGS. 41B, 41D, and 41F-41G of International Publication No. WO 2022/120219A1, which is incorporated by reference herein in its entirety), a progressive shift of MIEL signatures with age correlated with changes in chronological age was observed. The residual values from the training set (FIGS. 41C and 41F of International Publication No. WO 2022/120219A1, which is incorporated by reference herein in its entirety) were normally distributed and centered similar to the training set suggesting an ordered progression of heterochromatin changes in PBMC with age. Similar results (FIGS. 41D-41E and 41G of International Publication No. WO 2022/120219A1, which is incorporated by reference herein in its entirety) were found for the CD3+ T cell subset of the PBMCs.

Using the same H3K9me3 and H3K4me1 markers (FIGS. 42A-42E of International Publication No. WO 2022/120219A1, which is incorporated by reference herein in its entirety) and H4K20me3 mark (FIGS. 42F-42G of International Publication No. WO 2022/120219A1, which is incorporated by reference herein in its entirety) data from FIG. 41 of International Publication No. WO 2022/120219A1, which is incorporated by reference herein in its entirety, the ability of miBioAge to distinguish between cell chronological and biological ages without linear regression, i.e. unmanipulated (FIGS. 42A-42G of International Publication No. WO 2022/120219A1, which is incorporated by reference herein in its entirety), was demonstrated. Multidimensional Scaling (MDS) plots for total blood PBMC (FIG. 42B of International Publication No. WO 2022/120219A1, which is incorporated by reference herein in its entirety) and the CD3+ T cell subset (FIG. 42D of International Publication No. WO 2022/120219A1, which is incorporated by reference herein in its entirety) were generated. MDS1 and MDS2 denote the first and second dimensions obtained by Multi Dimensional Scaling (MDS) of the multidimensional space to the two-dimensional space for the purpose of visualization. FIGS. 42C and 42E of International Publication No. WO 2022/120219A1, which is incorporated by reference herein in its entirety, show Euclidian distance dot plot (after MDS). The distances from the center of all replicates for the youngest (53 days) and the oldest (681 days) mice were considered the reference points for Y axes “young” and X axes “old” mice respectively. Subsequently, Euclidian distances from the centers of young (53 days) and old (681 days) mice were calculated for centroids of all other ages/data points. The distances were calculated in the multidimensional space (˜550 dimension) then scaled to 2 dimensions and plotted using young-old axes. FIGS. 42F-42G of International Publication No. WO 2022/120219A1, which is incorporated by reference herein in its entirety, show scatter plots of unmanipulated H4K20me3-based signatures against chronological age and (insert in the right lower corner) quadratic discriminant analysis confusion matrices based on the validation set accurately (100%) separating signatures of young (Y) vs middle age (M) vs old (O) PBMC and CD3+ T cells. Correlation analysis and tradeline were implemented with Xlstat pack-age (Base, v19.06). These results are consistent with miBioAge providing a measurement of biological age without any computational manipulation of miBioAge epigenetic signatures, which therefore reflect the unmanipulated epigenetic landscape characteristic for the cells of individual animals.

MIEL-CLOCK (mibioAge) Quantitation of Biological Aging

The unmanipulated approach to miBioAge can also be used to show shifts in biological age in response to small molecule treatment and then used for to comparing, correlating, and cross-validating multiparametric signatures from miBioAge and from genomic-based approaches validated by classical sequencing-based genomic analyses done in parallel (FIG. 42 of International Publication No. WO 2022/120219A1, which is incorporated by reference herein in its entirety). This can be shown by employing classical treatments such as doxorubicin (DOX), paclitaxel (PTX), or temozolomide (TMZ), known to promote hallmarks of aging to compare miBioAge readouts in blood (PBMC and CD3+ cells) and liver hepatocytes to chronologically matched untreated controls. Blood was collected on day 0 (retro-orbital) from 3-month old C57BL/6J male mice and 22-month old C57BL/6J male mice. On day 1, doxorubicin was injected into the experimental mice. All controls received PBS injection. Blood was collected on day 20 (retro-orbital) from all mice and liver hepatocytes on day 21. Cells were analyzed 21 days after treatment to avoid the effects of acute stress response and favor the aging-related phenomena. DOX treatment has shifted the miBioAge signatures in liver hepatocytes towards the old age (FIG. 43A of International Publication No. WO 2022/120219A1, which is incorporated by reference herein in its entirety). A trend shift of miBioAge signatures in PBMC (FIGS. 43B-43C of International Publication No. WO 2022/120219A1, which is incorporated by reference herein in its entirety) and CD3+ cells (FIGS. 43D-43E of International Publication No. WO 2022/120219A1, which is incorporated by reference herein in its entirety) from DOX treatment mice towards that of the old animals was observed.

The effect of PTX and TMZ to cells is also tested using miBioAge via the experimental protocol used to test the effect of DOX provided herein. PTX is among the most widely used chemotherapy drug for the treatment of breast cancer, colorectal cancer, and squamous cell carcinoma of urinary bladder, and TMZ is the frontline treatment for several types of brain tumors. These drugs have different mechanism of action, however, all of them have been shown to increase the number of senescent cells, which is one of the hallmarks of aging. DOX intercalates within DNA base pairs, causing breakage of DNA strands and inhibition of both DNA and RNA synthesis. Paclitaxel-treated cells have defects in mitotic spindle assembly, chromosome segregation, and cell division. Chemotherapeutic effect of TMZ depends on its ability to alkylate/methylate DNA, triggering the death of tumor cells. The diversity of mechanisms helps generalize the utility of miBioAge to measure acceleration of biological aging following chemotherapy.

Genome wide changes in gene expression and chromatin accessibility acquired in liver hepatocytes with age were identified by conducting RNA-seq and ATAC-seq 71B37 experiments (FIGS. 44F-44D of International Publication No. WO 2022/120219A1, which is incorporated by reference herein in its entirety) using 7-month (young) and 24-month (old) C57BL/6J mice. Differential gene expression analysis identified a cluster of genes upregulated in old liver hepatocytes (FIG. 44F of International Publication No. WO 2022/120219A1, which is incorporated by reference herein in its entirety), which included interferon stimulated genes (ISG) that are canonically down-stream in the type I IFN and STAT1 signaling. A subset of such genes was confirmed by qPCR analyses using independent biological samples (FIG. 44G of International Publication No. WO 2022/120219A1, which is incorporated by reference herein in its entirety). To link chromatin dynamics changes to gene expression changes, ChIP-seq and ATAC-seq were conducted using young and old liver hepatocytes. Gene expression analysis suggested increased activity of enhancers bound by STAT1 in the old mice and the activation of IFN/STAT1 pathway in hepatocytes of old mice compared to young (FIGS. 44H-44I of International Publication No. WO 2022/120219A1, which is incorporated by reference herein in its entirety).

Such results indicate that miBioAge captures multiparametric signatures of young and old cells (e.g., PBMC, CD3+ T cells, splenocytes, hepatocytes, etc.) to distinguish cells of different ages based on imaging of epigenetic marks of heterochromatin. miBioAge can be used for quantitating the acceleration of measured biological age following the treatment with doxorubicin and similar age-affecting compounds.

In addition, it was observed that cultured fibroblasts (passaged 3-4 times over the period of 2 weeks in the dish) from young and old mice could no longer be distinguished using miBioAge. This could represent the “erasure” phenomenon that removes the distinct biological age-related epigenetic patterns upon culturing in the dish.

### Example 10: SENO-MIEL

Recent advances in understanding the biology of aging have raised the prospect of drug interventions to promote healthy aging in humans. Cellular senescence is a bona fide tumor suppression mechanism but also a cause of cell and tissue aging. Senescence is caused by a range of cellular stresses and characterized by an irreversible proliferation arrest and a potent pro-inflammatory phenotype, the senescence-associated secretory phenotype (SASP).

In this example, MIEL approach could be used to identify epigenetic changes during etoposides induced senescence (FIG. 45 of International Publication No. WO 2022/120219A1, which is incorporated by reference herein in its entirety). Curiously, the dynamics of epigenetic changes measured using histone acetylation differs from the dynamic measured using histone methylation (FIG. 45 of International Publication No. WO 2022/120219A1, which is incorporated by reference herein in its entirety). The changes from the baseline, measured using H3K9ac and H4K16ac, peak at day 1, plateau during the next 6 days, and then continue to increase on day 10. The changes from the baseline, measured using H3K9me2/3, H3K27me3, and H4K20me3 raise gradually, peak on day 4, then mostly decrease by day 10. Curiously, this dynamic of epigenetic changes is different from the well-studied changes in classical markers of senescence such as gH2AX, H2A1, HIRA, HP1g, and PML.

### Example 11: MIEL Rejuvenation

MIEL-Rejuvenation is the MIEL platform application to identify pro-longevity/healthy aging compounds based on their effect on epigenetic signature. Recent advances in understanding the biology of aging have raised the prospect of drug interventions to promote healthy aging in humans. However, such interventions should have essentially no toxicity or side effects. In practice, the throughput of drug testing (currently conducted in animals) is one of the major limitations for identifying interventions to promote healthy aging. Hence, a challenge is to identify candidate drugs able to induce rejuvenation and/or healthy aging, with minimal side effects in a high throughput fashion.

To meet this challenge, techniques described herein were employed to analyze the effects of various drugs on the analysis of epigenome topography at the single cell level. Because of large numbers of cells required for drug screening, mice were used as a source of (C57BLACK/6) of liver hepatocytes (˜98% pure in the preparations) for probing whether epigenetic signature of aging is retained after short-term cultures of hepatocytes. Indeed, MIEL analysis robustly distinguished young primary hepatocytes from old primary hepatocytes even after 3 days of culture (FIG. 45A of International Publication No. WO 2022/120219A1, which is incorporated by reference herein in its entirety). The ability of hepatocytes to maintain age-related epigenetic or multiparametric signatures in cell culture conditions provides the foundation for high throughput screening.

To validate the MIEL platform's ability to detect changes in epigenetic signatures or multiparametric signatures induced by small molecules and drugs, old hepatocytes (20-22 months) were treated with the rapamycin analog OSI-027 (FIG. 46B of International Publication No. WO 2022/120219A1, which is incorporated by reference herein in its entirety) for three days. FIG. 46C of International Publication No. WO 2022/120219A1, which is incorporated by reference herein in its entirety, shows an MDS plot, and FIG. 46D of International Publication No. WO 2022/120219A1, which is incorporated by reference herein in its entirety, shows a Euclidean distance plot presented in young-old axes showing changes in epigenetic landscape in old cells after 3 days of OSI-027 treatment with indicated concentrations. This data shows that treating cells with 2 uM OSI-027 shifts the signature of old cells closer to that of young cells, indicating that rapamycin analog OSI-027 could act as a rejuvenating agent to the cells by reversing the biological age of the cells.

Further validating the MIEL platform for high throughput screening, the effect of well characterized reference compounds on epigenetic landscape in old hepatocytes was examined. Rapamycin, aspirin, and NDGA, all of which have a positive pro-longevity effect, were tested. Statistically significant dose-dependent shifts of epigenetic signature of old cells towards that of young cells were observed for all three compounds tested (FIG. 46E of International Publication No. WO 2022/120219A1, which is incorporated by reference herein in its entirety). These results provide evidence of the feasibility of MIEL-based screening for compounds promoting cellular rejuvenation.

MIEL-Rejuvenation High Throughput Drug Screening

A 384-well plate phenotypic screening platform was then developed of which was used to screen a library of over 1200 compounds to identify differentiation of patient-derived human glioblastoma. Serum and BMP4 were used as positive controls known to induce differentiation of glioblastoma into non-tumorigenic cell. The Z′-scores of the screen plates were 0.66 and 0.78 for discriminating between untreated cells and either BMP4- or serum-treated cells, respectively. The Signal/Noise for the positive control BMP4 and serum treatments was 11.8 and 16.7, respectively. Multiple compounds were classified as hits (2.1% hit rate), 20 of which belong to 4 functional categories. 2 top compounds were selected from each category and directly compared the rankings based on MIEL and whole-genome expression analysis (19,000 genes FPKM>1). Excellent correlation was observed between the two approaches (Pearson correlation coefficient R=0.93, p<0.001, unpaired two-tailed t-test) confirming for the feasibility of MIEL-based high content screening.

Further screens expanded the set of ITP-selected compounds considered as positive controls by including estradiol, metformin, Nicotinamide mononucleotide (NMN), Nicotinamide Riboside (NR), and RG108. Additional screens utilized five multiplexed readouts in 384-well format: immunofluorescence labeling for H3K9me3, H3K27me3, DAPI, quantification of mean number of cell nuclei/field as a measure of general cell viability, and quantification of mean number of pyknotic nuclei as a measure of apoptosis at assay endpoint. The multiplex assay format provides mutually confirmatory data at the assay endpoint, reduces assay artifacts, and eliminates compounds inducing cell death/apoptosis. Such design enables a direct comparison of power and accuracy of MIEL assay based on H3K9me3, H3K27me3, and H4K20me3 using a set of reference compounds.

These high content screenings using MIEL pipeline were conducted to adapt these assay conditions. Briefly, on day 0, 10,000 primary mouse hepatocytes per well were seeded in 384 well plates. Hepatocytes were isolated via a 2-step perfusion method yielding ˜98% pure population of primary hepatocytes. At 24 hrs after seeding, compounds were added in DMSO. Primary mouse hepatocytes were determined to tolerate DMSO concentrations of at least 0.1% (v/v) without adverse effects on cell growth or viability over 5 days (compared to no DMSO control). Using the Echo acoustic compound dispensing system and 10 mM stock concentration library plates, each of the drugs at concentrations up to 10 μM was sufficiently delivered. At 4 days after seeding, cells were fixed and immunolabeled with antibody to H3K9me3, H3K27me3, and H4K20me3 (initially in separate wells) to determine the efficiency of each individual mark and potential for synergy.

The variation of miBioAge values from individual cells are distributed around the center (centroid) of a given biological entity (e.g., cell population, a subset of tissue, organ, organism). Such distribution provides an estimation of variance for a given biological entity (e.g., cell population, tissue, organ, organism). An important example of miBioAge quantification is an entropy, for instance a Shannon entropy, which is one of the features among several Haralick texture features computed for each image based on a pattern of epigenetic marks. Thus, MIEL value or MILE-CLOCK value includes a quantified value of computed entropy, for instance a Shannon entropy. Variance of miBioAge values, (e.g., variance of Shannon entropy) represents a valuable and informative measurement of epigenome homeostasis, or “epigenostasis.”

Age increases heterogeneity among individuals, organs, tissues, cell populations, for most if not all biological measurements. In other words, the variance among biological entities is increased for most if not all measurements derived from such biological entities.

miBioAge provides a tool to measure epigenetic heterogeneity or epigenostasis at single cell level. MIEL data have demonstrated the loss of epigenostasis and increase of epigenetic heterogeneity at single cell level with age and age-promoting perturbations. Conversely, the epigenostasis is restored and epigenetic heterogeneity is decreased as a result of health-span and life-span promoting interventions.

Therefore, epigenostasis measured by miBioAge provides additional/orthogonal quantitation of biological aging and the processes that affect biological aging at single cell level amenable for identifying epigenome rejuvenating compounds.

### Example 12: MIEL-EpiTox

MIEL-EpiTox is the MIEL platform application to identify epigenetically active environmental compounds. Applications of MIEL-EpiTox includes pre-screening newly developing compounds for testing epigenetic toxicity using human iPSC-based models. For instance, dozens of well-defined differentiation processes governing iPSC into neural, muscle, skin, gut, hematopoietic, pancreatic, and other cellular fates have been described. Inducing epigenetic alterations during such differentiation in the dish is likely to be a red flag and an indication that such compounds will be altering either development of adult cells or the function of adult cells.

MIEL-EpiTox can quantify epigenetic perturbations in human induced pluripotent stem cell (iPSC)-derived NPCs (iPSC-NPCs) after exposure to environmental chemicals to identify epitoxic compounds and to examine their effects on NPC self-renewal and differentiation in vitro, with the goal of developing a practical approach for risk-based decision making (FIG. 47 of International Publication No. WO 2022/120219A1, which is incorporated by reference herein in its entirety).

Cells were treated with test compounds, then fixed and immunolabeled for epigenetic marks. The pattern of marks within each nucleus was analyzed to create multivariate signatures or “fingerprints” of each perturbation. Since the technique analyzes texture features rather than intensities and morphologies, culturing and immunostaining artifacts are reduced. MIEL is related to “cell painting”, in which multivariate analysis of multiplexed biomarkers was used to classify protein localization, cell subpopulations, and drug mechanisms of action. The multiparametric signatures of the cells (centroids) were plotted in 2D via multidimensional scaling (MDS), and support vector machine (SVM) classification was used to establish borders to divide the epigenetic signatures into distinct classes. MIEL was employed to probe the presence of epitoxic compounds in the EPA ToxCast Phase I/II+elk library and examined the effects of 352 compounds on primary human fNPCs (Thermo-Fisher, #A15654). Cells were incubated with 10 μM compound for 72 h, labeled with fluorescence-tagged antibodies to H3K27ac and H3K27me3, and imaged on a Vala Sciences IC200 cytometer. Features were derived using Acapella (Perkin Elmer) high-content imaging and analysis software. SVM classification was conducted as previously described. The MIEL readouts (distances between the compounds and DMSO vehicle) and cell counts were z scored, scaled, and plotted, revealing 3 major classes of compounds: epitoxic, cytotoxic, and inert (FIG. 48A of International Publication No. WO 2022/120219A1, which is incorporated by reference herein in its entirety). To minimize false discovery, compounds were considered epigenetically “active” if the z-scores were >3 standard deviations from the average of DMSO controls (z-score>3, p<0.001, assuming normal distribution). The same criteria were applied to determine toxicity. Compounds with cell count z-scores between −3 and +3 and with a distance from the DMSO z-score>3 were considered epigenetically active (epitoxic) and non-toxic (green, FIG. 48 of International Publication No. WO 2022/120219A1, which is incorporated by reference herein in its entirety). Note that the epigenetic perturbations induced by exposure to epitoxic compounds did not correlate with cell counts (R=0.003, FIG. 48A of International Publication No. WO 2022/120219A1, which is incorporated by reference herein in its entirety). In contrast, epigenetic alterations and cell counts were correlated for the compounds identified as toxic (R=−0.78, FIG. 48A of International Publication No. WO 2022/120219A1, which is incorporated by reference herein in its entirety). The absolute levels of epigenetic marks were not altered by epitoxic compounds and did not correlate with the MIEL distance (FIG. 48B of International Publication No. WO 2022/120219A1, which is incorporated by reference herein in its entirety, inset) further underscoring the utility of epigenetic patterns detected by MIEL as opposed to trivial thresholding detection methods. These experiments identified 24 cytotoxic and 25 epitoxic compounds, providing proof of principle for the approach and an estimate of the frequency of compounds in the ToxCast Phase I/II+elk library altering epigenetic landscape in human NPCs. Among the epitoxic hits producing the largest epigenetic perturbations were 2,4-D 1-butyl ester, an HPV herbicide discontinued in 1972 due to high toxicity; fabesetron hydrochloride, a 5-HT3 receptor antagonist and close analog of ramosetron, which is widely used for the treatment of nausea and vomiting; and thalidomide, an infamous teratogen that was previously linked to NDDs. Hydroquinone, spironolactone, and dexamethasone sulfate, were singled out, for which information describing the administration regimen, dosage, and biological half-life are publicly available. All of these chemicals are known to readily cross the placenta and could be administered to pregnant women (Pregnancy Category C). It was estimated that following repeated administration to women (estimating 50-75 kg body weight), these compounds could reach concentrations that approach or exceed 10 μM, the concentration used in the studies. These results establish the existence of epitoxic compounds in the ToxCast Phase I/II+elk library and estimate the frequency of such compounds at 5-7%.

### Example 13 MIEL For Aging Testing

To provide biological age (ageotypes) at baseline and upon interventions/experiences (e.g. diet, exercise, sleep, etc.,). Notably, miBioAge is 100-1000 times more cost effective compared to DNA methylation clock, e.g. using microarray detection technology. See FIGS. 49-54 of International Publication No. WO 2022/120219A1, which is incorporated by reference herein in its entirety. FIG. 49 of International Publication No. WO 2022/120219A1, which is incorporated by reference herein in its entirety shows comparison between the MIEL and DNA methylation approaches. The diagram on the left illustrates the notion of biological vs chronological aging. Measurements related to serum analytes, deficit or frailty indices, or DNA methylation marks (DNA methylation clock) for particular chronological age are distributed so that some individuals resemble chronologically younger individual. The deviation from the mean values for a given chronological point are suggested to reflect biological age. FIG. 50 of International Publication No. WO 2022/120219A1, which is incorporated by reference herein in its entirety shows miBioAge epigenetic signature separates young vs old cells. The young cells (53 days) are predominantly located on the left side of the diagram (i.e., <0 on the x-axis), and the old cells (968 days) are exclusively located on the right side of the diagram (i.e., >0 on the x-axis). FIG. 51 of International Publication No. WO 2022/120219A1, which is incorporated by reference herein in its entirety shows miBioAge separates multiple young and old cell types. FIG. 52 of International Publication No. WO 2022/120219A1, which is incorporated by reference herein in its entirety shows unmanipulated miBioAge in the blood (PBMC and CD3+ T Cells). FIG. 53 of International Publication No. WO 2022/120219A1, which is incorporated by reference herein in its entirety shows PCA of Texture feature values separates age and CD3+vs CD3− cells. FIG. 54 of International Publication No. WO 2022/120219A1, which is incorporated by reference herein in its entirety shows Chemotherapy (DOX) and caloric restriction (CR) shift miBioAge signatures in different directions. Chemotherapy is known to accelerate aging, and the 3-month DOX increases the miBioAge distance compared to 3-month control (i.e., towards the 24-month control). On the contrary, 7-month caloric restriction (CR) decreases the miBioAge distance compared to the 7-month control (i.e., towards the 3-month control, and away from 24-month control).

It is contemplated that miBioAge and VITA can be used to test existing drugs/chemicals (e.g. A approved/Phase III clinical trials drugs) for their effect on biological age. See FIGS. 52-54 and FIG. 60 of International Publication No. WO 2022/120219A1, which is incorporated by reference herein in its entirety. It is contemplated that miBioAge and VITA can be used to test existing drugs/chemicals for their differential effect on young and old cells. See FIG. 66 of International Publication No. WO 2022/120219A1, which is incorporated by reference herein in its entirety. It is contemplated that miBioAge and VITA can be used to test existing senescence affecting (senolytic) molecules that are currently being developed. See FIG. 57 of International Publication No. WO 2022/120219A1, which is incorporated by reference herein in its entirety. It is contemplated that miBioAge and VITA can be used to test existing chemotherapy/environmental chemicals (ToxCast III ˜4700 compounds). It is important to determine which environmental compounds accelerate aging and to which extend. This will help to prioritize compounds which do not accelerate aging, eliminate the most potent ones, and design appropriate antidotes. It is contemplated that miBioAge and VITA can be used to discover new molecules that affect biological age through the high content high throughput screening in 384/15356 well plates. This assay will provide candidate hits to be tested and confirmed using orthogonal approaches including in vivo testing in mammalian systems. See FIGS. 61-66 of International Publication No. WO 2022/120219A1, which is incorporated by reference herein in its entirety.

Aging-associated diseases: atherosclerosis and cardiovascular disease, cancer, arthritis, cataracts, osteoporosis, type 2 diabetes, hypertension and Alzheimer's disease. The incidence of all of these diseases increases exponentially with age. FIGS. 55-56 of International Publication No. WO 2022/120219A1, which is incorporated by reference herein in its entirety, shows Variance of chromatin and epigenetic landscape (VITA) increases exponentially with age. FIG. 57 of International Publication No. WO 2022/120219A1, which is incorporated by reference herein in its entirety, shows variance separates age and different cell types. FIG. 58 of International Publication No. WO 2022/120219A1, which is incorporated by reference herein in its entirety, shows epigenetic marks increases with chronological age of cells. FIG. 59 of International Publication No. WO 2022/120219A1, which is incorporated by reference herein in its entirety, shows maximum/individual lifespan prediction from the drop of blood. FIG. 60 of International Publication No. WO 2022/120219A1, which is incorporated by reference herein in its entirety, shows DOX/CR shift VITA score in the different directions. DOX increases VITA score while CR decreases VITA score. FIG. 61 of International Publication No. WO 2022/120219A1, which is incorporated by reference herein in its entirety, shows high throughput screening using primary mouse hepatocytes. The miBioAge distance for young is shorter than that of old cells. FIG. 62 of International Publication No. WO 2022/120219A1, which is incorporated by reference herein in its entirety, shows screening quantification using rapamycin as an example. FIG. 63 of International Publication No. WO 2022/120219A1, which is incorporated by reference herein in its entirety, shows positive controls (17 beta-estradiol, NDGA, and Metformin). FIG. 64 of International Publication No. WO 2022/120219A1, which is incorporated by reference herein in its entirety, shows inactive molecules (Nicotiamide mononucleotide, nicotinamide riboside, and RG108). FIG. 65 of International Publication No. WO 2022/120219A1, which is incorporated by reference herein in its entirety, shows hits to be tested in animal models (INK128, NVP-BEZ235, and GSK2126458). FIG. 66 of International Publication No. WO 2022/120219A1, which is incorporated by reference herein in its entirety, shows differential effect of drugs on young vs old cells. FIG. 67 of International Publication No. WO 2022/120219A1, which is incorporated by reference herein in its entirety, shows MIEL detects epigenetic signatures/trajectories of senescence cells.

It is contemplated that applications of MIEL technology to quantitate epigenetic landscape in primary cells/nuclei isolated from different organisms (e.g., plants, microorganism, animals, including mammals, e.g., rodents, mice, human, and non-human primates). Such approach termed microscopic imaging of Biological Age (miBioAge) provide a readout/quantitate biological age at single cell level. miBioAge is increased following interventions that accelerate aging (e.g. chemotherapy). Therefore, miBioAge could be used to monitor the adverse interventions for human health, for instance through the analysis of blood/PBMC. Moreover, miBioAge is decreased following interventions that slow down aging (e.g. caloric restriction). Therefore, miBioAge could be used to monitor the beneficial interventions for human health, for instance through the analysis of blood/PBMC. Moreover, miBioAge does not require classical linear regression or elastic networks (which is the only way to build DNA methylation clock). Such multiparametric signatures could also be used to approximate chronological aging of single cells, tissues, organs, and organisms. Such multiparametric signatures are computed from the images of epigenetic landscape (e.g., H3K9me3, H3K9ac, H3K27me3, H3K27ac, H3K4me1, H4K20me3, DAPI, DNA methylation, and many others). Using the combination of epigenetic marks enhances the resolution and the power of epigenetic signature. miBioAge quantitates epigenetic landscape at single cell level and enables screening for small molecules that shift the signatures of old cells (e.g., hepatocytes) towards that of the young cells of the same tissue or organ. Such compounds are candidate hits to be tested in the in vivo setting for their ability to improve the healthspan and extend the lifespan. Several Intervention Treatment Program (ITP) compounds selected by NIA/NIH as previously documented to extend healthy aging and longevity, when applied to primary old mouse hepatocytes induce changes in epigenetic signature of these old cells that make them look more like mouse hepatocytes isolated from young animals.

### Example 14 Co-Occurrence. Of Epigenetic Marks (CINEMA)

FIG. 68 of International Publication No. WO 2022/120219A1, which is incorporated by reference herein in its entirety, shows quantification of co-occurrence using joint probability map. FIG. 69 of International Publication No. WO 2022/120219A1, which is incorporated by reference herein in its entirety, shows quantification of co-occurrence using joint probability map. FIG. 70 of International Publication No. WO 2022/120219A1, which is incorporated by reference herein in its entirety, shows experimental settings. FIG. 71 of International Publication No. WO 2022/120219A1, which is incorporated by reference herein in its entirety, shows co-occurrence matrix of DAPI, H3K27ac, and H3K27me3. FIG. 72 of International Publication No. WO 2022/120219A1, which is incorporated by reference herein in its entirety, shows differences in distributions of pixel values Aurora vs DMSO. FIG. 73 of International Publication No. WO 2022/120219A1, which is incorporated by reference herein in its entirety, shows differences in distributions of pixel values Aurora vs DMSO. FIG. 74 of International Publication No. WO 2022/120219A1, which is incorporated by reference herein in its entirety, shows differences in distributions of pixel values Aurora vs DMSO. FIG. 75 of International Publication No. WO 2022/120219A1, which is incorporated by reference herein in its entirety, shows TrialRun training and validation datasets. FIG. 75 of International Publication No. WO 2022/120219A1, which is incorporated by reference herein in its entirety, shows a schematic diagram of a typical analysis. Cells are segmented then 30% is randomly picked for final testing, whereas 70% is used for training/model construction. FIG. 76 of International Publication No. WO 2022/120219A1, which is incorporated by reference herein in its entirety, shows distributions for the training and validation: Aurora. 77 of International Publication No. WO 2022/120219A1, which is incorporated by reference herein in its entirety, shows distributions for the training and validation: Aurora kinase example. FIG. 78 of International Publication No. WO 2022/120219A1, which is incorporated by reference herein in its entirety, shows differences of probabilistic distributions. FIG. 79 of International Publication No. WO 2022/120219A1, which is incorporated by reference herein in its entirety, shows an example of the distributions of differences based on bivariate co-occurrence. FIG. 80 of International Publication No. WO 2022/120219A1, which is incorporated by reference herein in its entirety, shows co-occurrence analysis based on biviriate joint probability maps accurately classify most but not all 24 functional classes. FIG. 81 of International Publication No. WO 2022/120219A1, which is incorporated by reference herein in its entirety, shows co-occurrence analysis based on trivariate joint probability maps result 100% accuracy of separation of all 24 functional classes. In FIG. 81 of International Publication No. WO 2022/120219A1, which is incorporated by reference herein in its entirety, the x and y-axis show the training (reference) and validation (compared ones, “unknown”) compounds, respectively. The distances are log 10 transformed for better visualization. Green squares on the diagonal have the shortest distance (highest log 10 value). The sensitivity of the classification is greatly increased when Trivariate maps are used compared to bivariate resulting in 100% accuracy in the classification of all functional categories.

### Example 15 Genetically Engineered Epigenetic Probes (GEEPs)+MIEL-LIVE

Genetically Engineered Epigenetic Probes (GEEPs) are designed to visualize and report the pattern (including in 3D) of epigenetic modifications (both protein modifications and DNA modifications) through diverse imaging modalities. The patterns of epigenetic modifications reported by GEEPs could be used to compute epigenetic landscape from the same cell(s) over period of time, such approach is termed MIEL-LIVE. GEEP approach provides a unique advantage of continuous monitoring of epigenetic landscape over time at single cell resolution. In contrast, all current methods destroy the live cell constituents in the process of accessing the epigenetic information (ChIP-seq, ATAC-seq, Hi-seq etc.,). Combined with GEEP-based imaging, MIEL-LIVE analysis enables computing signatures of individual live cells over time and thus determine dynamics of epigenetic landscapes in single cells. Computing individual cell signatures derived from epigenetic landscapes enabled us to distinguish human glioblastoma cells dividing under the self-renewing vs differentiation conditions. Shannon entropy is used as a measure of information contained in an image.

$${E}_{s} = - {\sum }_{i} {\sum }_{j} {p}_{d} ( i , j ) ln {p}_{d} ( i , j ) {p}_{d} ( i , j ) = ( i , j ) th elements of normalized GLCM$$

Shannon entropy computed with epigenetic landscape of human glioblastoma cells engineered with GEEP dividing under the self-renewing conditions is significantly lower than the Shannon entropy computed with epigenetic landscape of human glioblastoma cells engineered with GEEP dividing under the differentiation conditions. Taken at face value this means that GEEP/MIEL-LIVE combination is capable of distinguishing self-renewing cell divisions (previously shown to be largely symmetric) from differentiation-associated divisions (previously shown to often have asymmetric components).

FIG. 82 of International Publication No. WO 2022/120219A1, which is incorporated by reference herein in its entirety, shows genetically encoded epigenetic probes (GEEPs) for live imaging of the epigenetic landscape. Live imaging of epigenetic landscapes, instead of antibody labeling in fixed samples, is a fundamentally different approach that opens up the possibility of acquiring dynamic real-time readouts of epigenetic landscapes. From the HTS prospective, this approach offers the possibility of direct and continuous image acquisition, which lowers dramatically (at least 10 times) the cost of HTS screening. To engineer GEEPs, previously characterized MPP8 chromo domain (1) or AF9 yeats bromo domain (2) is fused with red fluorescent protein (RFP) (3) and nuclear localization sequence (NLS) as shown in FIG. 82A of International Publication No. WO 2022/120219A1, which is incorporated by reference herein in its entirety. These cassettes were engineered as lentiviral vectors and delivered into primary human GBM cells. An example of live images showing the same cell before, during and after mitosis is shown in FIG. 82B of International Publication No. WO 2022/120219A1, which is incorporated by reference herein in its entirety. Note the disappearance of heterochromatic foci before mitosis. This example demonstrates the power of live imaging approach for discovering epigenetic phenomena that are difficult to document using conventional antibody labeling in fixed cells. To begin characterizing GEEPs, the cells engineered with H3K9me3 and H3K9ac reporters with the antibodies specific to the same or opposite mark were fixed and immunolabeled (FIG. 82C of International Publication No. WO 2022/120219A1, which is incorporated by reference herein in its entirety). Antibody immunolabeling for the H3K9ac mark correlated better with H3K9ac reporter (Pearson correlation coefficient R=0.57) compared to H3K9me3 reporter (R=0.25). Similarly, antibody for H3K9me3 correlated better with H3K9me3 reporter (R=0.62) compared to H3K9ac reporter (R=0.04). These preliminary results suggest that GEEP signals grossly overlap the corresponding antibody-based immunofluorescence signal. FIG. 82D of International Publication No. WO 2022/120219A1, which is incorporated by reference herein in its entirety, shows comparison of epigenetic perturbations induced by 1-day exposure to several drug classes using immunolabeling of PFA fixed cells and images acquired from GEEP-engineered cells revealed high correlation between both techniques, suggesting that GEEPs are suitable for MIEL analysis of drug-induced perturbation.

FIG. 83 of International Publication No. WO 2022/120219A1, which is incorporated by reference herein in its entirety, shows MPP8-TurboRFP GEEP reporting H3K9me3 in live 293T cells. Note the disappearance of granular pattern (flattening of epigenetic landscape) in all nuclei shortly before division. FIG. 84 of International Publication No. WO 2022/120219A1, which is incorporated by reference herein in its entirety, shows dynamics of single cell signatures (MEILL-signatures) based on epigenetic landscape of MPP8-TurboRFP. Dot plot of cell trajectories in 2 dimensions following multidimensional scaling from −250 dimensions of texture features (Haralick+TAS features). Three hypothetical cells (green, yellow, and blue) could behave differently over time according to stochastic or deterministic models. The red dot represents average position of three cells integrated over time. The experiment (right image) revealed that cells occupy distinct territories—four cells (blue, green, violet and red) are shown over several hours of observations. This enables tracking individual cell position/territories over time. FIG. 85 of International Publication No. WO 2022/120219A1, which is incorporated by reference herein in its entirety, shows single cell time lapse tracking under different proliferation conditions. MIEL-signatures and distanced between pairs of daughter cells were computed for each cell over time of observation. Changes in distance between the daughter cells cultured with Growth Factors (GF)=self-renewing conditions and with serum=differentiation conditions. FIG. 86 of International Publication No. WO 2022/120219A1, which is incorporated by reference herein in its entirety, shows average distances between pairs of daughter cells and all daughter cells under different growth conditions. MIEL-signatures and distanced between pairs of daughter cells were computed for each cell over time of observation. Changes in distance between the daughter cells cultured with Growth Factors (GF)=self-renewing conditions and with serum=differentiation conditions. FIG. 87 of International Publication No. WO 2022/120219A1, which is incorporated by reference herein in its entirety, shows Shannon entropy of an image. FIG. 88 of International Publication No. WO 2022/120219A1, which is incorporated by reference herein in its entirety, shows increase in Shannon entropy of epigenetic landscape with differentiation. Same as above, but only Shannon entropy (one of Haralick features) was considered to provide correlation with a more intuitive and understandable measurement/feature. FIG. 89 of International Publication No. WO 2022/120219A1, which is incorporated by reference herein in its entirety, shows Overarching idea for universal cancer cure strategy. Reversal of continuous self-renewal could be measured by MIEL-LIVE and thus could be used for a drug screening approach. Such approach would exploit a universal property of cancer cells—uncontrolled self-renewal. FIG. 90 of International Publication No. WO 2022/120219A1, which is incorporated by reference herein in its entirety, shows multiple marks measured simultaneously—statistics for relative position and abundance of epigenetic marks. Mapping enhancer and other biologically relevant chromatin entities. 3D MIEL and 3D MIEL-LIVE.

In sum, instead of using antibody labeling in fixed samples, you can use genetically encoded epigenetic probes (GEEPs) and image the cells over a period of time. Then, basically using the same techniques of image analysis and computer learning (in this case applying Shannon entropy as a measure of information contained in an image) one can detect changes in cellular differentiation and thus screen for anti-cancer or anti-aging drugs.

While preferred instances of the present disclosure have been shown and described herein, it will be obvious to those skilled in the art that such instances are provided by way of example only. Numerous variations, changes, and substitutions will now occur to those skilled in the art without departing from the disclosure. It should be understood that various alternatives to the instances of the disclosure described herein may be employed in practicing the disclosure. It is intended that the following claims define the scope of the disclosure and that methods and structures within the scope of these claims and their equivalents be covered thereby.

What is claimed is:

1. A method of determining a biological age of a primary cell at a single-cell level, the method comprising:
assaying a first plurality of primary cells and a second plurality of primary cells to detect expression patterns of a plurality of epigenetic marks, wherein the first plurality of primary cells are associated with a first chronological age, and wherein the second plurality of primary cells are associated with a second chronological age;
determining multiparametric signatures of the first plurality of primary cells and the second plurality of primary cells, based at least in part on the detected expression patterns of the plurality of epigenetic marks;
computer processing the multiparametric signatures of the first plurality of primary cells and the multiparametric signatures of the second plurality of primary cells, , wherein the computer processing comprises plotting against the first chronological age and the second chronological age; and
determining the biological age of the primary cell, based at least in part on the computer processing.

2. The method of claim 1, wherein detecting the expression patterns of the plurality of epigenetic marks in the first plurality of primary cells and the second plurality of primary cells further comprises detecting expression patterns of a plurality of epigenetic marks in a first nucleus of a first cell in the first plurality of primary cells and in a second nucleus of a second cell in the second plurality of primary cells.

3. The method of claim 1, wherein the assaying further comprises detecting a chromatin shape, detecting a deoxyribonucleic acid (DNA) modification, detecting a nuclear staining pattern, detecting a histone modification, detecting one or more genetically encoded epigenetic probes, or a combination thereof.

4. The method of claim 1, wherein the multiparametric signatures comprise a texture-associated feature.

5. The method of claim 4, wherein the texture-associated feature comprises Haralick texture features, threshold adjacency statistics, Gabor related features, radial features, or a combination thereof.

6. The method of claim 1, further comprising computer processing the texture-associated feature using a subcellular feature analysis, a machine learning feature extraction algorithm, a machine learning algorithm, or a combination thereof.

7. The method of claim 6, wherein the machine learning algorithm comprises a member selected from the group consisting of a support vector machine, a support vector regression, a linear regression, a quadratic discriminant analysis, a neural network, and a combination thereof.

8. The method of claim 7, wherein the machine learning algorithm comprises the quadratic discriminant analysis, and wherein the method further comprises using the multiparametric signature of the first plurality of primary cells to distinguish a cell population of the first plurality of primary cells from multiple cell populations.

9. The method of claim 7, wherein the machine learning algorithm comprises the support vector machine, and wherein the method further comprises using the multiparametric signature of the first plurality of primary cells to identify a character of the first plurality of primary cells in a single-cell population.

10. The method of claim 1, wherein the computer processing further comprises determining a first centroid of a first element of the multiparametric signature of the first plurality of primary cells, and a second centroid of the first element of the multiparametric signature of the second plurality of primary cells.

11. The method of claim 10, further comprising calculating a first multivariant centroid of the first plurality of primary cells and a second multivariant centroid of the second plurality of primary cells.

12. The method of claim 1, further comprising performing a data dimensionality reduction algorithm on the detected expression patterns.

13. The method of claim 12, wherein the detected expression pattern comprises 3-dimensional topological distribution, and wherein the data dimensionality reduction algorithm further comprises interpreting the 3-dimensional topological distribution as a two-dimensional projection using a multidimensional scaling.

14. The method of claim 1, wherein the first plurality of primary cells and the second plurality of primary cells comprise a same cell type.

15. The method of claim 1, wherein the first plurality of primary cells and the second plurality of primary cells comprise a different cell type.

16. The method of claim 1, wherein the first plurality of primary cells or the second plurality of primary cells comprises a hepatocyte, a fibroblast, a peripheral blood mononuclear cell, or an immune cell.

17. The method of claim 1, wherein the assaying further comprises capturing a series of images of the first plurality of primary cells and the second plurality of primary cells over a period of time.

18. The method of claim 17, wherein capturing the series of images further comprises capturing at least one image of the first plurality of primary cells and the second plurality of primary cells before, during, and after mitosis.

19. A method of determining an effect of a treatment on a primary cell, comprising:
applying the treatment to the primary cell, to obtain a treated primary cell;
detecting expression patterns of a plurality of epigenetic marks in the treated primary cell and an untreated primary cell;
determining a multiparametric signature of the treated primary cell and the untreated primary cell, based at least in part on the detected expression patterns of the plurality of epigenetic marks;
comparing the multiparametric signature of the treated primary cell to the multiparametric signature of the untreated primary cell; and
determining the effect of the treatment on the primary cell, based at least in part on the comparing.

20. A method of determining an aging, a residual lifespan, or a maximum lifespan of a biological entity, comprising:
detecting expression patterns of a plurality of epigenetic marks in a plurality of primary cells of a biological entity;
determining multiparametric signatures of the plurality of primary cells, based at least in part on the detected expression patterns of the plurality of epigenetic marks;
determining an average value of coefficient of variance of the multiparametric signatures of the plurality of primary cells; and
determining the aging, the residual lifespan, or the maximum lifespan of the biological entity based at least in part on the determined average value of the coefficient of variance of the multiparametric signatures.
